(19) United States
(12) Patent Application Publication
(10) Pub. No.: US 2012/0141562 A1
(43) Pub. Date: Jun. 7, 2012

(54) CELL COATED IMPLANTABLE DEVICE

(76) Inventors: Hardean E. Achneck, Durham, NC (US); George A. Truskey, Durham, NC (US); Ryan M. Jamiolkowski, Durham, NC (US)

(21) Appl. No.: 13/389,891

(22) PCT Filed: Aug. 12, 2010
(86) PCT No.: PCT/US10/02235

§ 371 Date: Feb. 10, 2012

Related U.S. Application Data
(60) Provisional application No. 61/272,054, filed on Aug. 12, 2009.

(51) Int. Cl. A61K 35/12; A61P 7/02; A61P 29/00; A61K 9/00

(52) U.S. Cl. 424/400; 424/93.7; 424/93.21

(57) ABSTRACT

The present invention relates, in general, to a cell-coated implantable medical device and, in particular, to an implantable medical device the blood-contacting surfaces of which are coated with endothelial progenitor cells (EPCs). In a preferred embodiment, the medical device is a titanium or titanium alloy-based medical device.

This application claims priority from U.S. Provisional Application No. 61/272,054, filed Aug. 12, 2009, the entire content of which is incorporated herein by reference.

### TECHNICAL FIELD

The present invention relates, in general, to a cell-coated implantable medical device and, in particular, to an implantable medical device, the blood-contacting surfaces of which are coated with endothelial progenitor cells (EPCs). In a preferred embodiment, the medical device is a titanium or titanium alloy-based device.

### BACKGROUND

Heart failure constitutes one of the fastest growing health problems with more than 550,000 newly diagnosed patients each year (Hunt, J. Am. Coll. Cardiol. 46(6):e1-e82 (2005)). Patients with severe heart failure refractory to medical treatment are best helped with cardiac transplantation. However, the demand for organs by far exceeds the supply with less than 2,200 donor organs available each year in the United States. At the point when patients are dependent upon continuous intravenous inotropic support, the survival may be as low as 6% within the next year (Hershberger et al, J. Card. Fail. 9(3):180-187 (2003)).

Those patients for whom a transplant is not available can be helped with mechanical assist devices. In the United States, it is estimated that 250,000 adult patients with end-stage heart failure per year would benefit from mechanical circulatory assist device (MCAD) implantation as an alternative to heart transplantation or as destination therapy (Lietz et al, Circulation 116(5):497-505 (2007), Lietz and Miller, Thorac. Cardiovasc. Surg. 20(3):225-233 (2008)). Most recently, mechanical assist devices have also been used for the treatment of children with heart failure refractory to medical therapy, and these devices will play an increasingly important role for this patient group in the future (Duncan, Asaio J. 52(6):e15-e21 (2006)).

The major causes of death in patients treated with MCADs are infection, bleeding and thromboembolic events (Deng et al, J. Heart Lung Transplant 24(9):1182-1187 (2005)). In order to minimize thromboembolic events, MCADs were initially developed with very smooth inner linings. However, focal and poorly adherent thrombus formation still occurred and often embolized. Subsequently, integrally textured surfaces were devised to encourage formation of a stable adherent biological lining covering the entire titanium blood-contacting surface area.

The HeartMateXVE by Thoratec (Pleasanton, Calif.) surface is made up of sintered titanium microspheres (50-75 μm) covering the rigid housing. This results in the formation of a densely adherent fibrin-cellular coagulum, which has been shown to decrease the risk of thromboemboli. Ultrastructural analysis of this coagulum has revealed compact fibrinous material with small aggregates of dense cellular tissue (Menconi et al, J. Cell Biochem. 57(3):557-573 (1995)). However, significant thrombin generation and fibrinolysis have been observed in patients with textured-surface MCADs causing a phenomenon of “compensated coagulopathy” (Spanier et al, J. Thorac. Cardiovasc. Surg. 112(4):1090-1097 (1996)). More recently, the progressive population of the MCAD surface with activated macrophages and monocytes expressing tissue factor and proinflammatory cytokines has been demonstrated, as has persistently elevated levels of tissue factor in the circulation (Spanier et al, J. Thorac. Cardiovasc. Surg. 118(3):404-413 (1999)). It has been concluded that either the device itself or the recruitment of inflammatory cells onto the MCAD surface promotes coagulation and inflammation.

Most MCADs have local areas of low flow and blood stasis. These regions are especially prone to thrombus formation. Because the mortality rate for infants awaiting a heart transplant is 40% and the number of donor organs is very limited, MCADs are now becoming a viable option for pediatric patients (Cooper et al, J. Biomech. Eng. 130(4):041019 (2008)). However, these devices operate at low flow and, therefore, are at very high risk of thromboembolism. MCAD design has thus focused on engineering a device that eliminates areas of stasis or low flow. This is achieved in the current HeartMateII device (Thoratec, Pleasanton, Calif.). However, this device is especially prone to cause bleeding complications. Geisen et al (Eur. J. Cardiothorac. Sur. 33(4):679-84 (2008), Epub Feb. 20, 2008) found evidence for an acquired von Willebrand Factor (vWF) disease in patients with this device and hypothesized that the high shear stress in the HeartMateII would render the vWF molecule dysfunctional. The artificial titanium surface of MCADs is also thought to sequester platelets and thereby cause the often observed postimplantation thrombocytopenia, which requires blood transfusions or a return to the operating room for bleeding (Scott-Burden et al, Circulation 98(19 Suppl):11339-11345 (1998)). Most patients with MCADs are also anticoagulated with warfarin and/or antiplatelet agents to prevent thrombotic events, which further exacerbates the risk for bleeding. Elimination of the need for these drugs could reduce costs and complications.

Geisen et al (Eur. J. Cardiothorac. Sur. 33(4):679-84 (2008), Epub Feb. 20, 2008) postulate that a change in MCAD design could overcome the problem of high shear damaging blood components. However, an MCAD operating at lower shear stress will again tip the pendulum of bleeding and clotting towards increased thrombotic complications.

The HeartMateIII assist device (Thoratec, Pleasanton, Calif.) (currently under development) consists of a titanium centrifugal pump with magnetic bearings, which may be more hemocompatible. However, the HeartMateIII has areas of low flow in the inlet and central core, with near zero velocity at the inlet elbow. These areas are, therefore, at risk for thrombus formation. Due to their relatively large volumes and low pump speeds, the Levacor device by WorldHeart (Oakland, Calif.) and DuraHeart by Terumo (Ann Arbor, Mich.) are similarly at risk for thromboemboli. This is also one of the major risk factors (and limitations) in the novel design of partial MCADs, such as the innovative Synergy's titanium micro-pump by CircuLite, (Saddle Brook, N.J.), which could benefit more than a million patients with less advanced heart failure by partially unloading the failing heart.

The goal of reducing high shear stress to reduce damage to blood components increases the risk of areas of stasis within the MCAD, favoring the formation of thromboemboli. In an effort to design better blood-contacting surfaces with anti-thrombotic qualities, the Berlin Heart Incor (Berlin Heart GmbH, Berlin, Germany) and the Dura Heart by Terumo (Terumo Cardiovascular Systems Corporation, Ann Arbor, Mich.) have heparin-coated inside surfaces. While MCADs coated with heparin reduce the risk of thrombosis, they pose the serious risk of heparin-induced thrombocytopenia in sensitized patients (Eghtesady et al, ASAIO J 51:820-5 (2005)).

In addition to bleeding and clotting, the interaction between the MCAD titanium surface and blood has been shown to cause a progressive defect in cellular immunity that renders patients at an increased risk of serious infections (Itescu and John, Ann. Thorac. Surc. 75(6 Suppl):558-565 (2003)). B-cell hyper-reactivity and dysregulated immunoglobulin synthesis can lead to allosensitization, which becomes a problem in patients for whom the MCAD is used as ‘bridge to transplant’ (Itescu and John, Ann. Thorac. Surc. 75(6 Suppl):558-565 (2003)).

The benefit of in vitro seeding artificial blood-contacting surfaces of femoropopliteal polytetrafluoroethylene bypass grafts with autologous endothelial cells was demonstrated more than a decade ago, when occlusion of such grafts was successfully reduced (Zilla et al, J. Vasc. Surg. 19(3):540-548 (1994), Leseche et al, Ann. Vasc. Surg. 9 Suppl:S15-S23 (1995), Magometschnigg et al, J. Vasc. Surg. 15(3):527-535 (1992), Deutsch et al, Surgery 126(5):847-855 (1999), Meinhart et al, Asaio J. 43(5):M515-M521 (1997)). However, all successes were hampered by the requirement to harvest the endothelial cells through an additional invasive procedure, such as harvesting a vessel.

The present invention addresses the bleeding and clotting problems described above by implantable devices (e.g., MCADs), particularly, titanium or titanium alloy-based implantable devices, the blood-contacting surfaces of which are coated with autologous endothelial progenitor cells (EPCs). EPCs suitable for use can be isolated, for example, from a simple peripheral blood draw or from umbilical cord blood (for application in pediatric patients). EPCs isolated from peripheral blood and umbilical cord blood have been rigorously characterized (Yoder et al, Blood 109(5):1801-1809 (2007); Mead et al, Curr Protoc Stem Cell Biol, Chapter 2:Unit 2C 1 (2008)) and have the potential for remodeling and repair, which are crucial given the stresses encountered at the inner surface of an MCAD. The properties of the EPCs can be enhanced by engineering them to express, for example, anti-thrombogenic genes.

### SUMMARY OF THE INVENTION

In general, the present invention relates to a cell-coated implantable medical device. More specifically, the invention relates to an implantable medical device (e.g., a titanium or titanium alloy-based device) the blood-contacting surfaces of which are coated with EPCs.

Objects and advantages of the present invention will be clear from the description that follows.

### BRIEF DESCRIPTION OF THE DRAWINGS

FIGS. 1A-1D. EPCs grow to a confluent monolayer on titanium, (FIG. 1A) SEM; 400× (FIG. 1B) Cytoplasm, eNOS (red); 200× (FIG. 1C) Nuclei, DAPI (blue); Cell Membrane, PECAM (green); 400× (FIG. 1D) Triple Stain (DAPI, eNOS, PECAM), 200×.

FIG. 2. Alamar blue reduction by EPCs on Ti and glass. EPCs on glass (blue), EPCs on titanium (purple), titanium without EPCs (yellow). (See also FIG. 30F.)

FIG. 3. Variable height flow chamber in perfusion circuit. (See also FIG. 37 which illustrates the same flow circuit.)

FIG. 4. EPC retention under increasing sheer stress (in dynes/cm²) with standard error.

FIGS. 5A and 5B. EPCs seeded on titanium slides and placed in a parallel plate flow chamber. (FIG. 5A) Before 48 hr flow, 20×. (FIG. 5B) After 48 hr flow, 20×.

FIG. 6. Production of NO by EPCs on titanium surface (n=1). (See also FIG. 33E which accurately depicts steadily increasing production of NO over time,)

FIGS. 7A and 7B. Platelet adhesion to uncoated (FIG. 7A) and EPC-coated (FIG. 7B) titanium surfaces.

FIGS. 8A-8D. (FIG. 8A) Ti sections and assembled Ti tube, upper row this design (arrow), lower row HeartMateII adapter piece (*). (FIGS. 8B-8D): seeded with this seeding device, (FIG. 8B) curved Ti at 40×, nuclei (white), middle part in focus only because of curvature, (FIG. 8C) curved Ti at higher mag. 400×, nuclei (blue), cell membrane (green), (FIG. 8D) curved Ti triple stain at 400×.

FIG. 9. Western blot of protein harvested from EPCs infected with Ad-TM, and two AAV-TM vectors: (1) Purified recombinant human TM. (2) Uninfected EPCs. (3) Adenoviral TM infected EPCs. (4) AAV2-TM infected EPCs. (5) AAV-SASTG TM infected EPCs.

FIG. 10. Example of the Fluency delivery system by Bard for stents. A denotes the location of the Nitinol stent struts which lie close together, D denotes the inner catheter which can be outfitted with macropores, E denotes the retractable outer sheath. This can be outfitted with micropores, K denotes a port that flushes the inner catheter lumen. All systems have such a port and the EPC solution can be delivered via this port.

FIG. 11. The solution containing EPCs can be introduced through the distal port of the delivery system (see K FIG. 10) into the inner catheter sheath.

FIG. 12. Delivery system the outer sheath of which is modified with micropores (about 10 μm diameter). A minimum cross-sectional dimension of 10 microns restricts flow through the pore. Ellipsoidal pores can allow a larger major dimension if the minor axis satisfies this requirement.

FIGS. 13A and 13B. Balloon expansion of Nitinol stents coated with EPCs. FIG. 13A Unsintered. FIG. 13B Sintered.

FIG. 14. Ti tube seeding chamber.

FIG. 15. Schematic of main components of an MCAD (shown is the example of an LVAD, such as the HeartMateIII, however, the general principles are equally applicable to other mechanical assist devices, including partial circulatory support devices)

FIG. 16. A hard shell encasing fully surrounding the entire LVAD.

FIG. 17. An encasing holding an LVAD, which is turned around its axis.

FIG. 18. An encased LVAD positioned inside a seeding device chamber.

FIG. 19. A sealing rotary unit.

FIG. 20. An sealing rotary unit can be connected to an LVAD (e.g., with tubing) and the sealing rotary unit can also be connected to a compartment around the driveline (formed by the encasing).

FIG. 21. An LVAD positioned inside a seeding device chamber, with a sealing rotary unit connected via tubing to different compartments.

FIG. 22. An LVAD shown from the outside suspended inside a seeding device chamber with a sealing rotary unit connected via tubing to different compartments.

FIG. 23. Addition of a rotary electric connector (aka Slip Ring).

FIG. 24. Connection of the axis of an LVAD to a motor.

FIG. 25. Addition of automated electric tubing clamps that close or open tubing as it leaves a sealing rotary unit.

FIGS. 26A-26D. EPC spreading on differentially pre-coated titanium surfaces after 8 hours. (FIG. 26A). With FN and serum (fn1s1). (FIG. 26B). With FN but no serum (fn1s0). (FIG. 26C). No FN but with serum (fn0s1). (FIG. 26D). No FN or serum (fn0s0).

FIG. 27. EPC spreading data.

FIG. 28. Atomic force microscopic image of 2 μm² sample area.

FIG. 29. 3-D surface topography of 0.25 μm² area from above surface. (See Also FIG. 34.)

FIGS. 30A-30 F. (FIG. 30A) Representative XPS spectrum of the HeartMate II Ti inner surface (14% Ti, 43% O₂, 41% C, 2% N). (FIG. 30B) XPS spectrum of Ti-coated slides prepared as described in Example 6 (22% Ti, 52% O₂, 23% C, 2% N). The carbon atom peaks and small nitrogen atom peaks in the XPS spectra reflect contaminations from the environment on the surfaces (Achneck et al, Microsc. Res. Tech. 73(1):71-76 (2010)). (FIG. 30C) SEM image of EPCs on Ti surface. (FIG. 30D) EPCs on Ti take up DiI-Ac-LDL (red). (FIG. 30E) EPCs on Ti stained for CD31 (green). Nuclei stained with DAPI (blue). Scale bar=100 μm for FIGS. 30C-30E. (FIG. 30F) EPCs' metabolic activity measured via the surrogate % reduction of the alamarBlue assay after long term culture (>1 week) on either FN-glass surfaces (red) or Ti surfaces (black). No significant difference was observed over the course of 14.5 hrs (p=0.42, n=6, mixed model).

FIGS. 31A and 31B. Platelets labeled with Cell Tracker Orange and incubated under identical conditions in a 1:1 solution of DMEM/Tyrodes Buffer (followed by 3 washes with buffered saline) on (FIG. 31A) bare Ti and (FIG. 31B) EPC-coated Ti (Cell Tracker Green). White arrow points toward group of PLTs on EPC-coated Ti. A>590-fold difference in platelet adhesion was observed in 5 images per slide and 3 independent experiments (p<0.0001). Scale bar=100 μm for both images.

FIGS. 32A-32F. (FIG. 32A) % EPC retention under 68 different (increasing) shear stresses after 15 min settling time on Ti followed by 5 min flow. Results grouped into 9 clusters. For each shear stress, 5 images at 100× magnification with approximately 100 cells per image were evaluated. EPCs were derived from 3 different pigs. Error bars indicate±1 SEM. (FIG. 32B) EPCs on Ti at initiation of flow, (FIG. 32C) 24 hrs, and (FIG. 32D) 48 hrs after initiation of flow at 15 dynes/cm². Area (FIG. 32E) and roundness (FIG. 32F) of EPCs on Ti after 0 hr, 24 hrs and 48 hrs of flow at 15 dynes/cm². Error bars indicate±1 SEM for E,F.

FIGS. 33A-33E (FIG. 33A) EPCs on Ti after 24 hrs culture and stained with Cell Tracker Orange. (FIG. 33B) Same field of view after 48 hrs flow at 100 dynes/cm². Scale bar=100 μm for both images. (FIG. 33C) Roundness of EPCs on Ti and FN-glass under static conditions, 15 dynes/cm² and 100 dynes/cm² shear stress. EPCs on Ti significantly elongated under 15 dynes/cm² (p<0.0001, n=500 cells per group, 2-Way ANOVA) and more so under 100 dynes/cm² (p=0.001, n=500 cells per group, 2-Way ANOVA). (FIG. 33D) The angle frequency distribution of EPCs on Ti at 15 dynes/cm²×48 hrs is significantly different from static conditions (p<0.0001, n=500 angles per group, KS-Test) and shear stress at 100 dynes/cm²×48 hrs (p<0.0001, n=500 angles per group, KS-Test). (FIG. 33E) Amount of nitrite (nmol) produced per 1×10⁶ EPCs as a surrogate for NO synthesized on Ti (solid lines) or FN-glass (interrupted lines) surfaces. Nitrite production was significantly larger under 15 dynes/cm² than under static conditions (p<0.0001), and under 100 dynes/cm² as compared to 15 dynes/cm² (p=0.0001), but not different between Ti and FN-glass surfaces (p=0.859). N=24 experiments, mixed model.

FIG. 34. Atomic force microscopy image of our Ti surface topography.

FIG. 35. EPCs on Ti stained for eNOS (red). Nuclei stained with DAPI (blue). Scale bar=100

FIGS. 36A-36D. Flow cytometry results. EPCs are negative for CD14 and CD45, but positive for CD31.

FIG. 37. Laminar flow circuit consisting of a peristaltic pump (Masterflex, Cole-Parmer), in-line with a pulse dampener (Cole Parmer), flow chamber (either parallel plate or variable height with a glass window to enable imaging EPCs on Ti inside the chamber) and 250 ml medium reservoir (Corning Incorporated).

FIGS. 38A and 38B. Human EPCs from a patient with coronary artery disease on Ti before flow (FIG. 38A) and after 48 hours of flow at 100 dynes/cm² (FIG. 38B). Images were obtained by transillumination of the 100 nm thick Ti layer on glass slides with an inverted Leica DMIL microscope with a Qicam camera and Leica QCapture software. The scale bars are=100 μm.

FIG. 39. Assembly and preparation of compound pure Ti tube for implantation.

FIG. 40. Loading of Ti tube with EPC-containing solution.

FIG. 41. Seeding of Ti tube with EPCs.

FIG. 42. Generation of confluent surface of EPCs.

FIG. 43. Ti tube in flow loop consisting of pulse dampener, reservoir and cardiopulmonary bypass pump.

FIG. 44. Implantation of EPC-coated Ti tube.

FIG. 45. Lumen of control Ti implant occluded by thrombosis.

FIG. 46. Clot-filled lumen of control Ti implant.

FIG. 47. Clot-free lumen of EPC-coated Ti implant.

FIG. 48. Fluorescent microscopy demonstrates presence of fluorescently labeled cells at the density seeded prior to implantation (40×).

FIG. 49. Spread of EPCs in vivo (200×).

FIG. 50. Side view of holes in stent. Holes fully penetrate outer sheath of delivery system and are in close proximity so that pressure is reduced.

FIG. 51. The roughness of the Ti tube inner surface was determined with a Zygol 3D Optical Profiler, Zygo New View 500. White light interferometry was used to produce a 3D topographical map of the Ti surface. 3 images per Ti sample were obtained and an average roughness (Ra)=350 μM calculated.

### DETAILED DESCRIPTION OF THE INVENTION

The present invention results, at least in part, from the realization that coating blood-contacting surfaces of titanium (Ti) or titanium alloy-based implantable medical devices with EPCs reduces thrombogenicity and increases biocompatibility. Immune rejection can be avoided by using EPCs derived from a patient's own blood.

The present invention relates to a cell-coated implantable medical device, to methods of coating the medical device, and to methods of treatment (including methods of treating vascular disease) based on the use of the cell-coated medical device. The cells used to coat blood-contacting surfaces of the medical device can be EPCs, advantageously, autologous EPCs, derived, for example, from peripheral blood, umbilical cord blood or from the placenta. The presence of EPCs on blood-contacting surfaces reduces the occurrence of restenosis and thrombosis at the site of implantation of the medical device. While EPCs (otherwise known as endothelial colony forming cells (ECFCs)) are preferred, the invention includes the use of endothelial cells (e.g., blood-derived endothelial cells) generally. For example, the invention includes the use of bone marrow-derived endothelial cells, adipose tissue-derived endothelial cells, microvascular-derived endothelial cells, vascular-derived endothelial cells, cardiac-derived endothelial cells, human brain-derived endothelial cells and skin-derived endothelial cells. The invention also includes the use of embryotic/fetal stem cells as well as cells genetically altered to adopt endothelial cell properties/phenotype.

Medical devices that can be cell (e.g., EPC)-coated in accordance with the invention include devices that are introduced, temporarily or permanently, into a mammal for the prophylaxis or therapy or diagnosis of a medical condition, as well as wireless monitoring of physiologic parameters. Such devices include, but are not limited to, an MCAD (e.g., a left ventricular assist device (LVAD), including its inflow and outflow cannula and adapters), an artificial heart, a vascular stent, a vena cava filter or other device to protect against pulmonary embolism, a nonvascular stent (e.g., gastrointestinal, pulmonary or biliary), a vascular graft or a hemodialysis graft, a dental implant, an orthopedic implant, a reconstructive prosthesis, a cardiac prosthesis (e.g., an artificial heart valve), a biological heart valve prosthesis (e.g., derived from animals such as pigs—xenografts can be coated with EPCs to render them more biocompatible and less thrombic), an implantable wireless biosensor, which measures parameters including, but not limited to, pH and blood oxygenation, blood pressure, blood glucose level (for application of blood sugar control in diabetics), an implantable insulin pump, an implantable artificial oxygenator, an implantable artificial kidney or filtration system, artificial or tissue engineered urinary bladder and/or ureter, other implantable artificial organ, implantable electric device (e.g., pacemaker), or wireless Micro-Electro-Mechanical System (MEMS). The medical device can be made, for example, of titanium or a titanium alloy, which includes shape memory alloys (e.g., Nitinol (NiTi), aluminum and vanadium alloys (Ti6Al4V) and (Ti6Al4V ELI), as well as niobium alloys (Ti6Al7Nb), iron alloys (Ti5Al2.5Fe), including, but not limited to, titanium alloys containing Nb, Ta, Zr, Mo, Fe, Si). The device can also be made of other metals, e.g., stainless steel.

EPCs suitable for use in the invention can be isolated from a patient's peripheral blood according to established protocols (Yoder et al, Blood 109(5):1801-1809 (2007)). Automated cell sorting systems can be utilized to effect fast and efficient cell isolation on a large scale basis. Droplet-based cell sorting constitutes one form of optical cell-sorting that has been used for research purposes (Herzenberg et al, Clin Chem 48:1819-27 (2002)). It allows for the sorting of cells, e.g., EPCs, based upon fluorescently labeled EPC-specific markers (e.g., CD31+, CD34+, CD14−, CD45−). Optical cell sorting makes it possible to simultaneously analyze signals from multiple fluorescent tags, as well as the size and morphology of the cells so that a decision can be made as to whether to accept or reject a cell.

Another method of automated cell sorting applicable to therapeutic settings is based on the use of magnetic antibodies. Cells with a given biomarker can be removed from a solution by a magnetic field. This method does not allow for the simultaneous detection of multiple markers (Dainiak et al, Adv Biochem Eng Biotechnol 106:1-18 (2007)). An alternative approach involves the utilization of a high speed optical cell sorting device, such as Gigasort, produced by Cytonome (Cytonome, Inc. Boston, Mass.), which optically sorts cells using fast switching microfluidic valves in an enclosed disposable container at a speed of 10⁹ cells/hour (DiGiusto et al, Cytotherapy 9:613-29 (2007)). Isolated EPCs can be expanded in culture using standard culture methodologies.

EPCs suitable for use in the invention can also be isolated from umbilical cord blood using the same procedures described above (Mead et al, Curr Protoc Stem Cell Biol Chapter 2:Unit 2C 1 (2008)) and expanded in culture. Another approach is to isolate EPCs with pooled human platelet lysate, as described by Reinisch et al (Blood 113(26):6716-6725 (2009)).

Isolated cells (e.g., EPCs) can be genetically engineered or molecularly modified to express factors or proteins that, for example, directly or indirectly inhibit thrombogenesis, restenosis or platelet adhesion, or that enhance cell viability or that have anti-inflammatory properties. For example, a vector (e.g., a viral vector, such as an adenoviral vector, an adeno-associated viral vector, an AAV chimeric vector or a retroviral vector or pseudotyped viral vector) can be constructed comprising an expression cassette containing a gene, pseudogene, mutant gene, such as dominant negative gene, or a gene silencing construct, e.g., shRNA or microRNA. Suitable expression cassettes can be constructed using an array of conventional cloning methods. While the use of gene delivery via viral vectors is preferred, non-viral methodologies can also be used, e.g., plasmid or cosmid DNA delivery via liposomal reagents, lipoplexes or polyplexes, electroporation, sonoporation, hydrodynamic gene delivery, use of a ‘gene gun’, and nucleofector techniques and nanoparticle delivery.

Cells (e.g., EPCs) are advantageously engineered to over-express thrombomodulin (TM) in view of its properties as an anticoagulant and as an anti-inflammatory molecule. TM participates in one of the most important anticoagulant feedback loops in blood coagulation, whereby the proteolytic activity of thrombin is harnessed to prevent further thrombin generation. Additionally, TM-induced generation of activated protein C exerts anti-inflammatory and anti-apoptotic functions on endothelial cells (Dahlback and Villoutreix, Arterioscler. Thromb. Vasc. Biol. 25(7):1311-1320 (2005)). While TM is preferred, the invention includes cells (e.g., EPCs) genetically engineered to express other polypeptides/proteins, including, but not limited to, the endothelial cell protein C receptor (EPCR), which increases the PC activation rate by thrombin-TM complexes (Fukudome et al, J Biol Chem 269:26486-91 (1994)); endothelial nitric oxide synthase, which synthesizes the L-arginine derivative nitric oxide and, therefore, acts as a potent platelet adhesion and aggregation inhibitor and reduces cytokine and endotoxin-induced expression of tissue factor (Yang et al, Circulation 101:2144-8 (2002)); heparin-like molecules, which through their interaction with antithrombin and heparin cofactor II counteract prothrombotic proteins (Stern et al, J Exp Med 162:1223-35 (1985)); subspecies of heparin cofactor II, which act by inhibiting the procoagulant thrombin molecule (Shirk et al, Arterioscler Thromb Vasc Biol 16:1138-46 (1996)); plasminogen activators (e.g., tissue plasminogen activator and urokinase-type plasminogen activator), which convert plasminogen to plasmin and, therefore, allow for fibrin breakdown; tissue factor pathway inhibitor, which inhibits the activated coagulation factor VIIa in combination with tissue factor (Osterud et al, Thromb Haemost 73:873-5 (1995)); annexin V, which, as a nonglycosylated protein with high affinity to negatively charged membrane phospholipids, acts by displacing coagulation factors and inhibiting platelet adhesion (He et al, J Biol Chem 283:19192-200 (2008)); prostacyclin (PGI2), which, through upregulating cyclic adenosine monophosphate, inhibits platelet aggregation (Willis et al, Lancet 2:682-3 (1986)); anti-inflammatory proteins, such as transforming growth factor-beta 1 (Smith et al, J Immunol 157:360-8 (1996)); Interleukin-10 (Mulligan et al, J Immunol 151:5666-74 (1993)); Interleukin-4 (Mulligan et al, J Immunol 151:5666-74 (1993)); high-density lipoprotein (Cockerill et al, Circulation 103:108-12 (2001)); Protein Z, which forms a complex with ZPI and binds to phospholipid surfaces where they inhibit activated factors IX, X and XI (Corral et al, Br. J. Haematol. 137:99-108 (2007)) and other proteins, factors and receptors involved in the anti-coagulation-, fibrinolytic- and anti-inflammatory pathways.

The genetic engineering of cells (e.g., EPCs) is not restricted to over-expressing or addition of advantageous genes, but also includes the inhibition, down-regulation and ‘knockout’ of disadvantageous genes, which include but are not limited to those encoding plasminogen activator inhibitor-1, which inhibits the action of plasminogen conversion to plasmin (Rijken et al, J Thromb Haemost 7:4-13 (2009)); tissue factor, which can be expressed on activated or damaged endothelial cells and plays a pivotal role in activation of the extrinsic coagulation pathway (Osterud et al, Thromb Haemost 73:873-5 (1995)); platelet activating factor receptors (Derian et al, Expert Opin Investig Drugs 12:209-2 (2003)); pro-inflammatory interleukins; and cell surface phospholipids, which enhance the adhesion of coagulation proteins, as well as other proteins, factors and receptors involved in the coagulation, anti-fibrinolytic and pro-inflammatory pathways.

Once the cells are genetically-altered by transfection of exogenous DNA or RNA expression cassettes comprising the desired gene(s), the cells can be grown using standard tissue culture techniques. Aliquots of cells that express and secrete desired genes can be stored frozen in liquid nitrogen using standard techniques. Frozen cells can be thawed and regrown using standard tissue culture protocols prior to use.

The present invention also relates to methods of coating blood-contacting surfaces of implantable devices with cells (e.g., EPCs) and to devices suitable for use in such methods. In order to increase the rate of cell spreading, blood-contacting surfaces of implantable devices can be pre-coated with an extracellular matrix protein, such as fibronectin, collagen, vitronectin, laminin, fibrin, or any of the following components containing molecules, proteins or constructs, including proteoglycans, such as heparan sulfate, chondroitin sulfate, keratin sulfate, or non-proteoglycan polysaccharide containing molecules, such as hyaluronic acid, or any combination thereof. Blood-contacting surfaces can also be pre-coated with gelatin or a gelatin matrix or gelatin foam, e.g., Gelfoam (Pharmacia and Upjohn, Pfizer), cellulose, microfibrillar collagen, thrombin, e.g., recombinant human thrombin (Recothrom, ZymoGenetics), a fibrin sealant, e.g. Tisseel (Baxter), or fibrin gel, fibrin glue, fibrinolytically inhibited fibrin glue, adhesive glue or sealant, hydrogel. Blood-contacting surfaces can also be pre-coated with a serum protein or other blood component, or growth factor or hormone, e.g., platelet-derived growth factor BB, basic fibroblast growth factor, acidic fibroblast growth factor, or transforming growth factor beta1. Pre-coating can also be effected using a synthetic polymer, e.g., polymer of lysine, ornithine or arginine, or polymethylmethacrylate, polyacrylic acid, or L-glutamic acid-treated construct, or glutaraldehyde-preserved cellular matrix, or a biodegradable binder or coating, such as poly(DL-lactide-co-glycolide), or biodegradable polyester, e.g., polyhydroxyalkanoate, polysorbate, or poly amino acids, e.g., poly-L-lysine, or chitosan, fetuin, or cationic silica microbeads, other types of microbeads or carbon-deposition surface coating, polyethyleneterephthalate with or without alteration by plasma discharge surface modification, or covalently-attached avidin, biotinylation, or RGD peptide sequence containing molecules, structures or constructs or peptides, which are cross-linked to RGD peptides, or molecules specific to one or more EPC specific integrin binding site or synergistic binding site, e.g., the amino acid sequence DRVPHSRN or antibodies, peptides or aptamers specific to EPCs, or any combination of the above. The aforementioned molecules can be physiosorbed or covalently bound to the underlying surface, e.g., titanium/titanium alloy surface, for the latter a variety of methods are available, including silanization (e.g., linkage of steel to an aminosilane crosslinker), biotinylation, covalent linkage to dopamine, etc. In addition to the proteins, molecules, polymers, structures and artificial constructs mentioned above, other cell types can be used to pre-coat the blood-contacting device surfaces to provide for a suitable matrix for EPCs, including, but not limited to, fibroblasts, smooth muscle cells, stem cells, mesothelial cells, mesenchymal cells, progenitor cells, myocytes, or other cell type. It is desirable to pre-coat the implantable device with autologous cells to avoid rejection. Fibroblasts, for example, can be easily harvested for this purpose from a sample of the patient's skin.

Example 4 describes the effect of pre-coating a titanium surface with FN on EPC spreading. A pre-coating can be also be applied to 316L stainless steel in order to attach EPCs to steel to coat, for example, coronary steel stents, such as the BiodivYiso™ AS (Biocompatibles Cardiovascular Inc. CA), BeStent™ (Medtronic Inc. Minnesota), CYPHER™ (Cordis Corporation, Fla.), NiRflex™ (Medinol Ltd. Israel), TAXUS™ EXPRESS2™ (Boston Scientific Corporation), Liberte™Monrail™ (Boston Scientific Corporation, Md.), or RithronXR (Biotronik GmbH, Germany) stent or cobalt chromium alloy stents, such as the MULTILINK-VISION™ (Guidant Corporation, CA) stent. A pre-coating matrix protein (or other component) can be inserted prior to cell seeding into the stent through macropores in the same fashion as the EPCs (through macropores in the guidewire sheath, as described in the Examples that follow) while the stent is in its undeployed state. Alternatively, the stent can be pre-packaged with the matrix protein already adsorbed or otherwise linked to the metal struts (in the case of a stent). In the latter case, the stent or other device can be maintained in a liquid or moist environment in its package, e.g., in DPBS.

In addition to the surface chemistry, surface microtopography can also play an important role in how well cells (e.g., EPCs) adsorb and attach (Boyan et al, Biomaterials 17:137-146 (1996)) to blood-contacting surfaces. The study described in Example 5 indicates smooth titanium surfaces are advantageous in terms of cell spreading and adhesion. Other metal surfaces can be converted to a smooth titanium surface using established methods, including titanium filament evaporation, electron-beam evaporation, and sputter deposition.

While very smooth surfaces can be advantageous, other possible implantable device blood-contacting surfaces include rougher surfaces that can be generated using a variety of techniques, including but not limited to, acid-etching, plasma electrolytic oxidation, laser ablation, bead blasting, sand blasting and the attachment of nanoparticles, such as titanium dioxide, gold, iron, and aluminum oxide, to the surfaces. Other techniques include chemical functionalization of the metal device surface followed by nanoparticle attachment; silanes, which can be attached to a variety of metal surfaces (Ti, Ni, Ta or steel), are commercially available with a variety of reactive groups that can be used to secure metal and/or inorganic nanoparticles to device surfaces rendering them rough. These reactive groups can interact with nanoparticles in a variety of ways including: through opposite charges, metal-ligand interactions, and/or chemically, e.g. EDC/NHS reaction (which can attach an acid functionalized particle to an amine containing surface).

Further, atom transfer radical polymerization can be used to add rough polymer coatings to the surface through surface initiated polymerization. In addition to a nanostructure being directly formed in the metal surface, it can also be created through shaping surface coatings. “Top-down” lithographic methods can be used for this purpose and include: optical lithography, X-ray lithography, electron-beam direct-write lithography, extreme ultraviolet lithography, charged particle lithography, nanoimprint lithography, scanning probe lithography such as dip-pen nanolithography, atomic force microscopic nanolithography, and magentolithography. “Bottom-up” methods are applicable as well and include Langmuir-Blodgett thin films, as well as electrostatic self-assembly, the alternate adsorption of anionic and cationic polyelectrolytes onto the substrate. Another “bottom-up” method that can be used for this purpose is the creation of self-assembled monolayers (SAMs). SAMs can be produced through physical vapor deposition techniques, electrodeposition, electroless desposition, or adsorption from solution. Synthetic chemistry creates the basic building blocks, and weaker intermolecular attractions bind the blocks together and form a nanoscale structure.

Cell-coating devices and methods suitable for use in the invention are described in the Examples that follow. In addition to the methods described in the Examples, other methods of seeding a stent or other tubular structure or hollow or cavernous device can be based on the use of magnetic forces, electrostatic forces, centrifugal forces and/or gravity dependent settlement.

In the case of magnetic forces as the cell seeding method, the stent lumen can be filled with cells (e.g., EPCs) that have been magnetized. Cells can be magnetized with magnetite cationic liposomes, e.g., cationic liposomes containing 10-nm magnetite nanoparticles, according to the methods of Ito et al (Tissue Eng 11:1553-61 (2005)), and Ino et al (Biotechnol Bioeng 102:882-90 (2009)). If the stent to be coated is made out of a ferromagnetic material, e.g. pure 316L stainless steel, it can be magnetized so that it attracts cells filled with magnetic nanoparticles. A ferromagnetic stent can be seeded in its undeployed form as illustrated in FIG. 11. Moreover, a ferromagnetic stent can be implanted using standard techniques, deployed in the desired location, and then seeded in vivo by introducing magnetic cells (e.g., EPCs) via the stent delivery device. In order to seed non-ferromagnetic stents, such as nitinol stents, a magnet/magnets can be placed around the outside of the stent/stent delivery device to generate a magnetic field and direct the cells circumferentially onto the inside surface of the stent. This process can be achieved while the stent is in its non-deployed form inside the delivery device.

In the case of electrostatic forces as the cell seeding method, the metal stent in its non-deployed state inside the sheath can be held at a positive voltage by connecting it to the positive terminal of a battery, such that it will attract the negatively charged cells (e.g., EPCs). The positive charge can be maintained for the duration of cell seeding. Additionally, the guidewire of the stent delivery system can be held at a negative (repulsive) charge during the seeding process.

In the case of centrifugal forces as the cell seeding method, the stent can be rotated around its longitudinal axis such that cells (e.g., EPCs) in a cell solution introduced into its lumen are subjected to centrifugal forces outward against the inner surface of the stent struts. For this purpose, the sheath, which contains the non-deployed stent, can be equipped with a joint capable of rotating 360 degrees around its axis and connected on the far end of the delivery device to a motor, that rotates the stent and thereby exerts the necessary centrifugal force. Alternatively, the sheath containing the non-deployed stent can be disconnected from the delivery device and rotated by an electric motor before re-connecting it to the sheath after seeding. Such a reconnection can be achieved through a quick snap design or a screw design. In the quick snap design, the sheath, which restrains the stent, can be endowed with a male connector that slides into the female end of the connector with a seamless transition on its inner and outer surfaces. The quick snap design can be modified such that both ends are locked together after they are connected. This prevents any accidental loss of the distal sheath inside the vasculature. A screw in design can be used for the same purpose. In this case, a preloaded hook or tab in either end can be added as a safety feature that allows the screw part to ‘lock’ into the bearing part after assembly.

The stent'can also be seeded through gravitational force alone. This, again, requires rotation of the stent containing sheath, either in conjunction with the entire delivery device, or separately, connected to the delivery device via a 360 degree joint, or entirely disconnected from the delivery system as described above. The ideal rotational speed for this method can be estimated by calculating the average settling time of cells (e.g., EPCs) assuming they are randomly distributed. For an EPC of density ρ_{c} submerged in media of density ρₘ and viscosity μ, located in the middle of a stent of radius R, the settling velocity vₛ can be expressed as vₛ=(2/9)×(R²/μ)×(ρ_{c}−ρₘ)_{g}, where g is the gravitational constant.

As shown in the Examples below, EPCs remain adherent to titanium under shear stresses significantly higher than those found in a blood vessel. Furthermore, after exposure to shear conditions comparable to those experienced by endothelium inside a human artery (15 dynes/cm²), EPCs align with their main axis coinciding with the direction of flow. Thus, EPCs coated on a titanium (or titanium alloy) surface remodel to form a lining with similar morphological properties to native endothelium. Moreover, EPCs seeded on titanium or a titanium alloy produce nitric oxide, as expected from normal healthy endothelium, as a response to stimulation by shear stress. Coating titanium or titanium alloy surfaces with EPCs significantly reduces platelet adhesion, an important antithrombotic property.

In accordance with the present invention, a patient suffering from heart failure and who has been identified as a candidate for implantation of a MCAD can undergo a simple blood draw in order to harvest the EPCs. The EPCs (which can be genetically engineered, as described above) can be expanded in culture and stored frozen. The MCAD can, for example, be introduced into an appropriate seeding device, a solution of the EPCs added to the seeding device and the MCAD thereby automatically seeded. At the time of surgery, the EPC-coated MCAD can be implanted into the patient from whom the EPCs were isolated.

The present invention can be used to address a major complication of cardiovascular stenting—‘in-stent restenosis’. After implantation of stents into the vasculature, 20%-80% re-occlude. The present invention provides a method of seeding the stents immediately prior to implantation with the patients' own EPCs, which generate an endothelial lining on the inside of the stent. Patients suffering from coronary or peripheral vascular disease can undergo a simple blood draw and EPCs can be isolated using standard techniques. The EPCs (which can be genetically engineered, as described above) can be expanded in culture using standard techniques and stored frozen. Stents can be seeded within minutes of implantation; the frozen EPCs can be thawed and the stent seeded within about 10 minutes. This approach is applicable to any stent in any vessel, e.g., coronary arteries, peripheral arteries and veins and vessels in the brain.

While the invention is described in detail with reference to titanium or titanium alloy implantable devices, the cell (e.g., EPC) coating procedure can also be used to address problems faced by patients suffering from end stage kidney failure that are only kept alive through dialysis. Often, a dialysis graft (typically made from PTFE) is implanted as a bridge between the patient's artery and vein in order to provide a site of access for hemodialysis. However, these artificial grafts can clot off over time and expensive and invasive procedures are necessary to remove the blood clots or surgically implant new grafts. The present invention can be used to reduce the risk of graft thrombosis by lining the inside of the graft with, for example, EPCs (e.g., genetically engineered EPCs). EPCs can be isolated from the patient's blood and grown in culture and frozen until use. The inside of the dialysis graft can be seeded within minutes immediately before implantation.

Subjects that can be treated using the medical devices of this invention are mammals, including, humans, dogs, cats, pigs, sheep, calves, rodents or monkeys.

Certain aspects of the invention are described in greater detail in the non-limiting Examples that follow.

### Example 1

The pig has a long-standing history as an established animal model for researching the biocompatibility of blood-contacting surfaces (Ueberrueck et al, J. Surg. Res. 124(2):305-311 (2005), Whelan et al, Heart 83(3):338-345 (2000)). Furthermore, the pig is one of the most accepted models for the study of coagulation biology and inflammation (Velik-Salchner et al, Thromb. Res. 117(5):597-602 (2006), Kang et al, Thromb. Haemost. 89(2):256-263 (2003), Roussi et al, Thromb. Res. 105(3):209-216 (2002), Salartash et al, Ann. Surg. 231(6):789-794 (2000), Dal Nogare et al, Am. Rev. Respir. Dis. 142(3):660-667 (1990), Fernandez and Volek, Nutr. Metab. (Lond) 3:17 (2006)). Yorkshire swine have the added advantage that the size of their inferior vena cava allows for implantation of titanium tubes, modeled in their dimensions after the HeartMateII cannula adapters (Thoratec Corporation, Pleasanton, Calif.). The fact that pigs' blood is slightly hypercoagulable as compared to humans' (Velik-Salchner et al, Thromb. Res. 117(5):597-602 (2006)) is a further advantage.

EPCs have been isolated from peripheral blood of Yorkshire swine following the method described by Yoder et al (Blood 109(5):1801-1809 (2007)). These cells have endothelial cell (EC) morphology by light and electron microscopy, and stain positive for typical EC markers by immunofluorescence, such as CD31, endothelial nitric oxide synthase (eNOS), BS-1 lectin, VEGF-R2, and vWF. In order to show that these EPCs are not the ‘early outgrowth’ EPCs, which express multiple EC markers, but have a limited proliferative potential and many characteristics of macrophages, they were evaluated with flow cytometry. Flow cytometric analysis was performed with a FACSCalibur flow cytometer (Becton, San Jose, Calif.). The negative results for CD14 and CD 45 confirmed the absence of hematopoietic or monocytic cells.

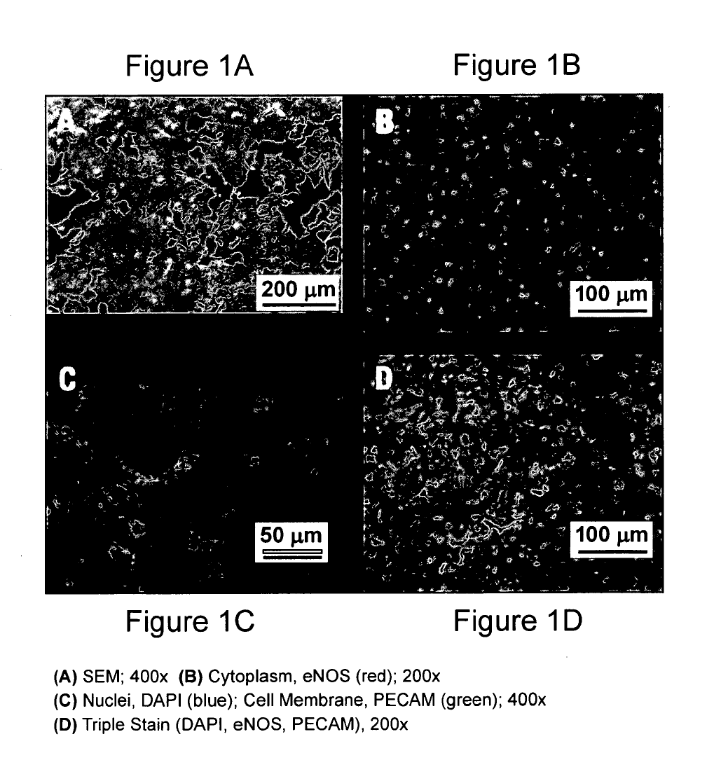

To be clinically useful, EPCs must be able to attach to titanium and grow to confluence on such surfaces. To investigate this, Ti slides were manufactured and seeded with EPCs. First, glass slides from Fisher Scientific (Pittsburgh, Pa.) were cleaned by immersion in a hot solution of 3:1 sulfuric acid:hydrogen peroxide for approximately 1 hour. After rinsing with deionized water, they were dried using nitrogen gas. 100 nm of titanium was then deposited on the glass slides at a rate of 2 Angström/s using a CHA Industries Solution E-beam Evaporation System (Fremont, Calif.). X-ray photoelectron spectroscopy was used to confirm that the surfaces were indeed pure titanium and identical to the blood contacting surfaces of mechanical assist devices. Ti slides were then sterilized by steam autoclaving at 120° C. for 30 min (AMSCO Scientific Series, Gravity Stage 3 Apex, NC). Scanning electron microscopy was used to determine the optimal seeding density of 150,000 cells/cm²(<passage 9)(see FIG. 1A). EPCs were then seeded onto the Ti slides below their optimal density in Endothelial Cell Basal Media 2 (Lonza, Clonetics, Walkersville, Md.) with EGM-2 Single Quots (Lonza, Clonetics, Walkersville, Md.) and 10% FBS (HyClone, Thermo Fisher Scientific, Logan, Utah). For human cells, 10% FBS was found to work well. For porcine EPCs, 2% porcine serum was a better choice for isolation and growth. FBS can be substituted with 5% albumin (preferably recombinant human albumin) or plasma free platelet lysate, or a combination thereof, to decrease the risk of known (e.g., Creutzfeldt-Jakob disease) and unknown communicable diseases (e.g., bovine). Ti slides were incubated at 37° C. and 5% CO₂ for 8 days. Ti slides were then stained and imaged using a Nikon Eclipse TE2000-U (Nikon, Tokyo, Japan) inverted fluorescent microscope with a digital camera (DS-Qi1Mc, Nikon) (see FIGS. 1B-1D). The results indicate that EPCs adhere to Ti and grow into a confluent layer fully covering the entire surface.

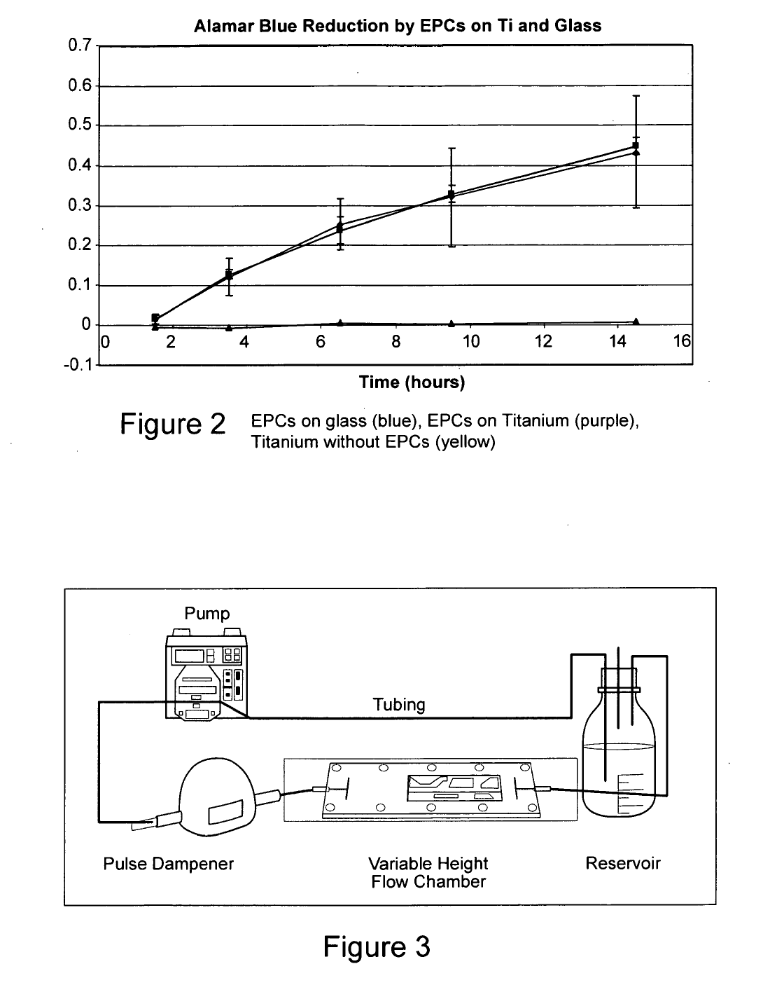

In order to assess the viability of seeded EPCs on titanium surfaces, the alamarBlue redox assay (Invitrogen, Carlsbad, Calif.) was used. As the EPCs grow, their innate metabolic activity results in a chemical reduction of alamarBlue (Invitrogen, Carlsbad, Calif.) from an oxidized form to a reduced form. The absorbance of alamarBlue (Invitrogen, Carlsbad, Calif.) added to EPC-coated and uncoated titanium (control) was measured and compared to EPC coated and uncoated glass microscope slides (Thermo Fisher Scientific, Pittsburgh, Pa.) (control). The percent reduction of alamarBlue (Invitrogen, Carlsbad, Calif.) was calculated and found to be very similar for the EPC-seeded titanium and for the EPC-seeded glass at 5 time points measured over the course of approximately 14 hours (see FIG. 2). These results show that EPCs on titanium are as viable and metabolically active as those growing on glass surfaces.

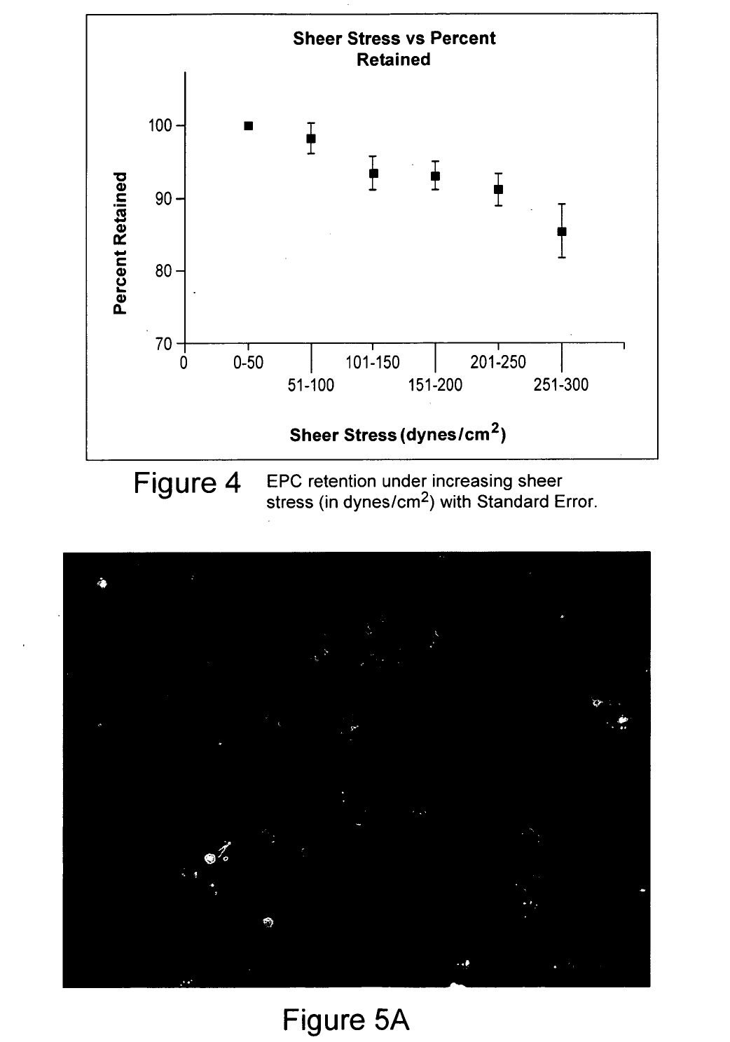

To determine whether EPCs would detach under flow conditions an ex-vivo circuit with a variable height flow chamber was engineered (Mathur et al, J. Biomed. Mater Res. A 64(1):155-163 (2003), Brown et al, Biomaterials 28(27):3928-3935 (2007)) (see FIG. 3). The variable height chamber made it possible to investigate EPC retention at several different shear stresses on a titanium slide for each given flow experiment. The wall shear stress acting on the EPCs was calculated using the following equation:

$${τ}_{w} = \frac{6 μ Q}{{wh}^{2}}$$

where τ is the shear stress in dynes/cm², μ denotes the viscosity (0.01 g cm/s for DPBS), Q the flow rate in ml/min, w the width of the channel (15 mm) and h its height (varying from 530 μm to 360 μm). To measure the strength of EPC adhesion to Ti slides, EPCs were labeled with the fluorescent cell tracker orange (Invitrogen Carlsbad, Calif.). This allowed for visualization of EPCs on the radiopaque Ti through the transparent top of the flow chamber, immediately before and after flow. After 2 rinses with DPBS, cells were lifted off by incubation with 0.025% Trypsin (Lonza, Clonetics, Walkersville, Md.) for 5 min and subsequent neutralization with Trypsin Neutralization Solution (Lonza, Clonetics, Walkersville, Md.). Cells (100,000 cells/ml DPBS) were pipetted onto a Ti slide in a quadri-PERM 4-Compartment Culture Dish (Greiner Bio One, Monroe, N.C.) and incubated for 15 min to allow for the cells to settle and attach on the titanium surface. The titanium slide was then placed into the flow chamber as described above and 5 images each in five positions along the longitudinal axis of the chamber were obtained at 10× magnification using phase contrast microscopy (Nikon Diaphot, Tokyo, Japan) immediately before and after flow. The perfusate consisted of DPBS and the flow ranged from 200 ml/min to 600 ml/min for a duration of 5 min. The shear stresses generated ranged from 46 dynes/cm² to as high as 313 dynes/cm². In comparison, the shear stress acting on an endothelial cell in the human and pig aorta is in the range of only 15-25 dynes/cm² and even less in the vena cava. The results indicate excellent EPC adhesion to titanium surfaces at shear stresses up to ten times of what they would encounter under physiologic conditions (see FIG. 4).

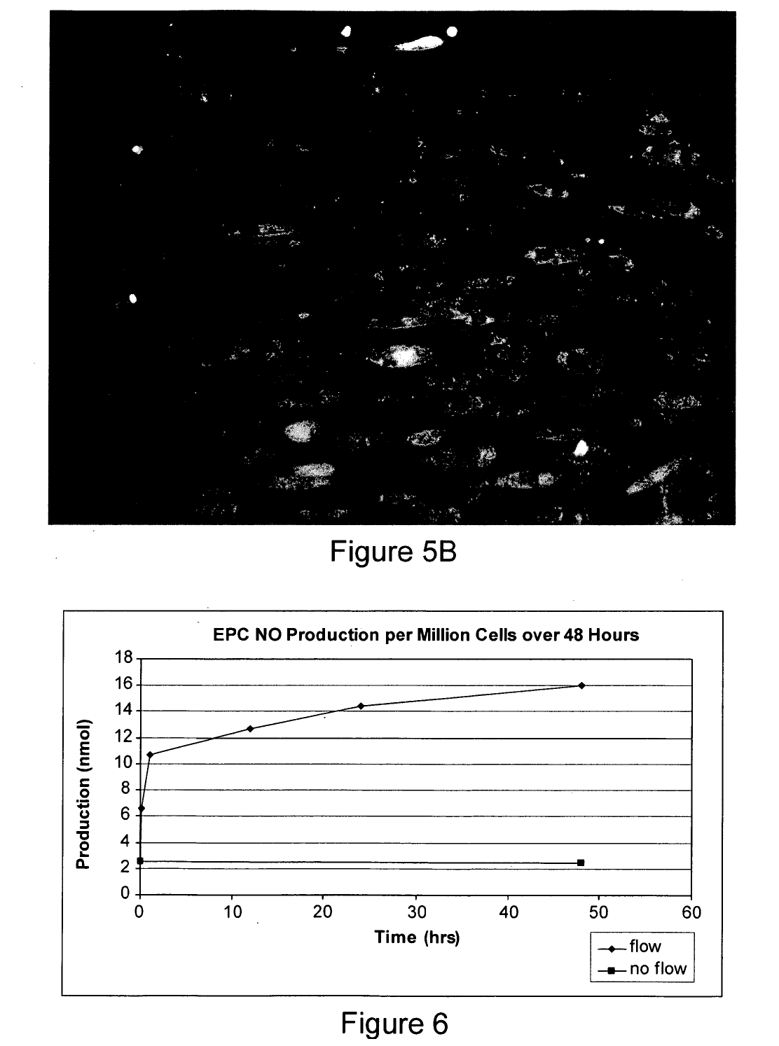

To assess long term cell adhesion and behavior to flow, EPCs were similarly labeled and seeded onto titanium slides and placed inside a parallel plate flow chamber at uniform height. Fifteen images per slide were obtained at 200× magnification before and after 48 hours of flow at a shear stress of 15 dynes/cm², comparable to what they would encounter in a coronary artery. The cell angle of their long axis with respect to the direction of flow was measured in all images using Leica software (Media Cybernetics, Image-Pro Plus Version 6.3, Bethesda, Md.). EPCs remained adherent in the form of a confluent monolayer for the duration the flow. Moreover, the cells elongate and orient themselves in the direction of the flow as compared to static conditions. Before flow, there was an approximately even distribution of cells with angles between 0 degrees and 90 degrees. After flow, nearly two thirds of the cells revealed an angle ranging from 0-10 degrees, and about nine tenths of the cells exhibited angles from 0-20 degrees, which demonstrates that EPCs on titanium have the ability to remodel and adapt to their environment (see FIGS. 5A and 5B).

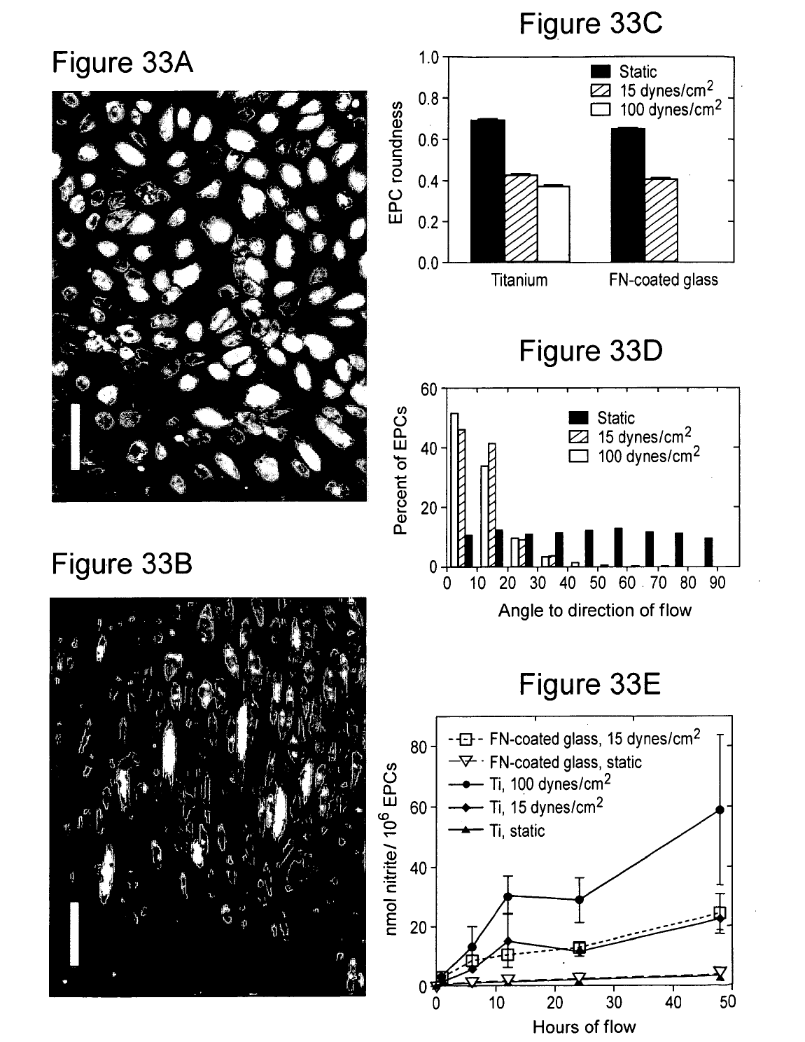

An important property of healthy endothelial cells is their ability to synthesize the antithrombogenic and vasodilatatory molecule nitric oxide in response to shear stress (Yang et al, Circulation 101:2144-8 (2000)). To evaluate EPCs' ability to produce NO on titanium surfaces, NO production was measured in the flow media using a Sievers Nitric Oxide Analyzer (GE Analytical Instruments, Boulder, Colo.) as a function of time under physiologic shear stress and compared to the NO production under static conditions. As shown in FIG. 6, EPCs respond to shear stress as expected for normal endothelium and produced 16 nmol NO per 1×10⁶EPCs under flow as compared to only 2.7 nmol NO per 1×10⁶ cells under static conditions. Subsequent studies that more accurately reflect NO production over time are described in FIG. 33E.

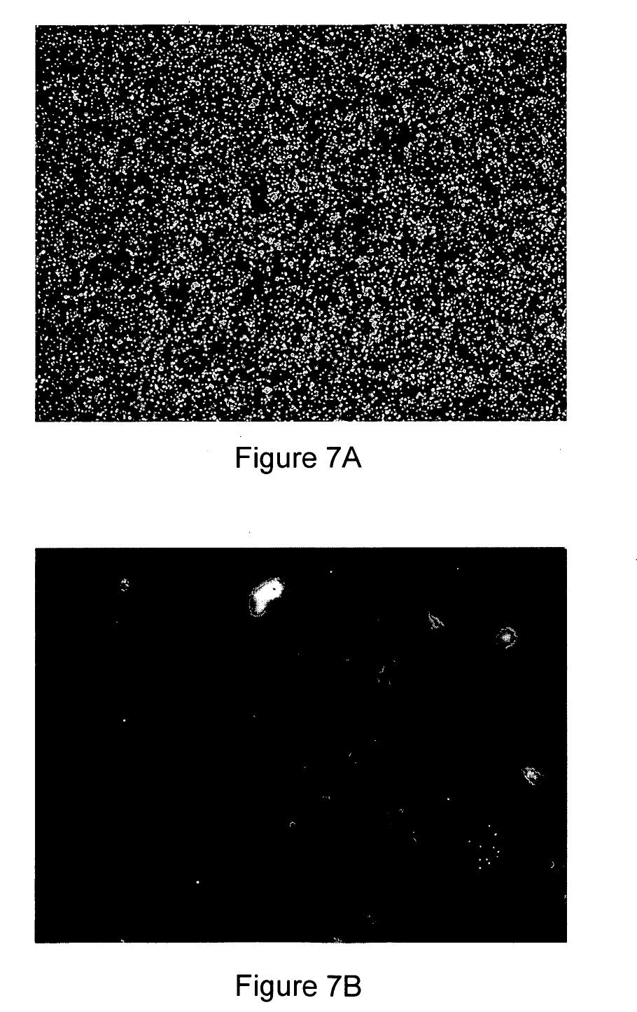

In order to assess the improved biocompatibility of EPC-coated titanium implants, a platelet assay was developed in analogy to the methods of Baker et al (Am J Hematol 56:17-25 (1997)). Porcine platelets were isolated from blood and labeled fluorescently, resuspended in a solution of buffered saline glucose citrate at 1×10⁹/ml and added to porcine EPC-coated and uncoated (control) titanium pieces in a 1:1 solution of Tyrode's Buffer and DMEM. Samples were then incubated for 10 min at 37° C. and 5% CO₂. Cell-coated and uncoated control samples were subsequently washed ×3 and imaged. A drastic difference in platelet adhesion between coated and uncoated samples was observed. Whereas only an average of approximately 4220 platelets per cm² adhered to the cell-coated surfaces, an average of approximately 2,500,000 platelets per cm² consistently adhered to uncoated titanium samples in repeat experiments under the same conditions. (FIGS. 7A and 7B).

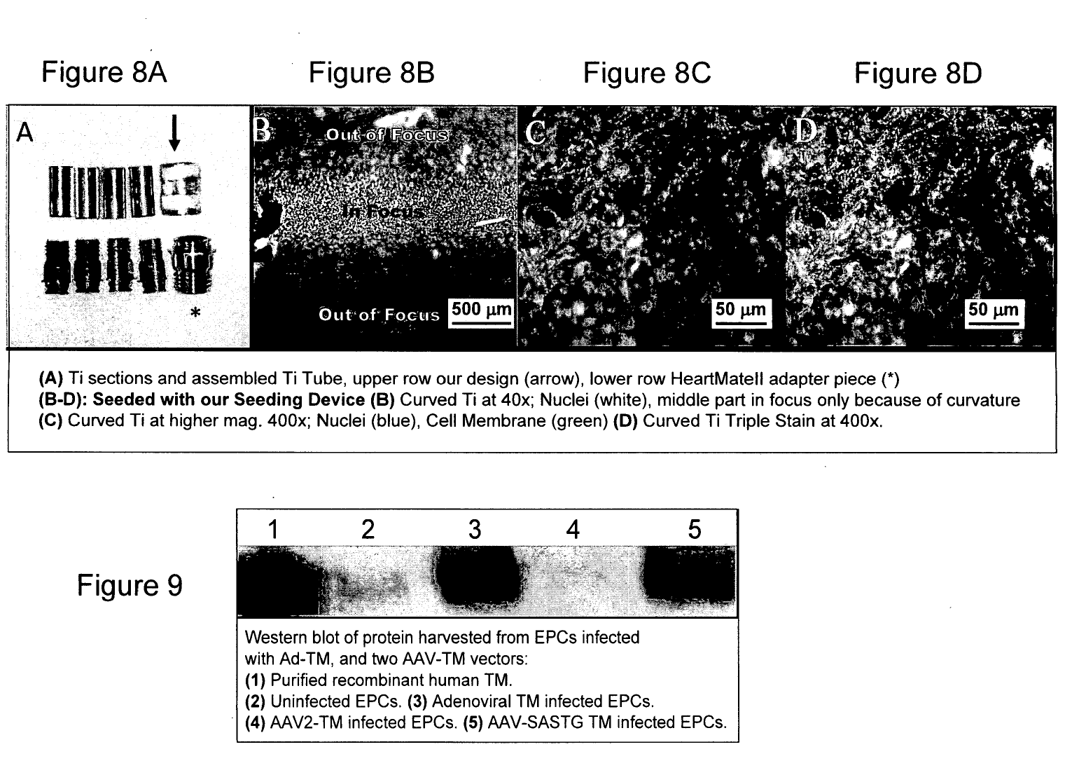

Studies were next undertaken to line a 3 dimensional titanium tube on its inside surface. In order to evaluate the success of such a method, it must be possible to image the inside surface of the titanium tube after cell seeding. This is also crucial for the animal experiments, so that the titanium tubes can be evaluated once the animals are sacrificed. This goal is achieved by precutting the titanium tube into four longitudinal sections and then re-assembling the pieces back into a tube (see FIG. 8A arrow). This was done by heat-shrinking two layers of medical grade polytetrafluoroethylene (PTFE) over the titanium pieces. PTFE monofilament suture (Gore-Tex, Gore, Flagstaff, Ariz.) was wrapped over the titanium tube between the two layers of PTFE and thus allows for suturing the inferior vena cava (IVC) wall to the outside of the titanium tube so that no false lumen exists when the Ti is inserted into the pig vessel. These titanium tubes are easily sterilized by steam autoclaving at 120° C. Whereas the titanium tubes were modeled in their dimensions after the 2 cm long HeartMateII adapter piece (Thoratec Corporation, Pleasanton, Calif.) (FIG. 8A*), 4 cm long titanium tubes will be engineered for implantation into the pig IVC to increase the blood contacting surface area.

The advantages of a fully confluent autologous EPC blood-contacting surface will be demonstrated in an animal model. In order to recognize the EPCs at the end of the study when the animals are sacrificed and their titanium tubes explanted, the cells will be labeled before seeding onto the titanium tubes. Qtracker Cell Labeling Kits (Molecular Probes, Invitrogen, Eugene, Oreg.) can be used. These kits are designed to load cells in culture with highly fluorescent nanocrystals. These nanocrystals that are taken up by cells and maintain their fluorescent properties for up to months in vivo. Moreover, in contrast to other methods of labeling cells, e.g., transfection with green fluorescent protein, these quantum dots are passed on to daughter cells in vivo and are less likely to evoke an immune or inflammatory reaction (Lin et al, BMC Biotechnol. 7:67 (2007), Rosen et al, Stem Cells 25(8):2128-2138 (2007)).

EPCs can be genetically modified to further enhance their antithrombogenic properties. The endothelial cell membrane contains a number of molecules that are critically important in the regulation of blood coagulation. Thrombomodulin (TM), a transmembrane protein expressed on quiescent endothelial cells, is one such regulatory molecule that tends to prevent clot formation by serving as a cofactor for the thrombin-catalyzed proteolytic activation of protein C. Activated protein C then catalyzes the inactivation of activated factors V and VIII, which are themselves cofactors for the activation of factor X and prothrombin. Based upon TM's properties as both an anticoagulant and anti-inflammatory molecule, it has been selected as the candidate gene to over-express in the EPCs.

Viral vectors offer greater transduction efficiency to cultured endothelial cells than nonviral methodologies, such as plasmid DNA delivery via liposomal reagents, electroporation, or nucleofector techniques. Both adenoviral (Ad) and adeno-associated viral vectors (AAV) expressing the human thrombomodulin (TM) transgene have been developed. The AAV-SASTG variant efficiently transduces the endothelial progenitor cells (see FIG. 9). Unfortunately, the human and porcine thrombomodulin amino acid sequences are quite divergent (69% identity), so much so, that it would be virtually impossible to utilize the human TM in a porcine model without the potential for immunological consequences. This is especially important since the biocompatibility of EPC coated implants will be evaluated by quantifying known markers of inflammation and thrombosis. Therefore, it will be necessary to reengineer a porcine version of the viral vector for use in the present application. Recently, the porcine TM gene has been cloned (Roussel et al, Am. J. Transplant 8(6):1101-1112 (2008)).

Although both AAV and Ad vectors are capable of transducing endothelial progenitor cells (see FIG. 9), the adeno-associated viral vector may be the better choice for this study, as AAV vectors offer the advantage of long term gene expression in animal models on the order of months to years. This time frame is compatible with the experimental design, in which it is proposed to assess cellular duration at 30 days post delivery. In contrast to AAV, in vivo gene expression arising from adenoviral vectors is eliminated or minimized by 30 days. In addition, in comparison to adenoviral vectors, AAV based vectors elicit minimal immune and inflammatory responses.

As the porcine TM AAV vector is unavailable commercially, its development is required. During the first step in the development of the AAV vector, the open reading frame of interest will be cloned into the AAV cis plasmid between the cytomegalovirus promoter enhancer and SV40 polyA site. The entire expression cassette is flanked by the AAV inverted terminal repeats, which are necessary for packaging of the vector genome into the viral capsid. AAV vectors are produced in bulk in HEK293 cells or 293 cell variants, such as HL1 using NIH Biosafety Level 2 (BSL2) criteria. Large scale AAV preparations will be manufactured by the triple transfection of 293 cells with three plasmids: i) the AAV cis plasmid, which contains the expression cassette, which will be packaged into the AAV capsid, ii) the AAV trans-plasmid, which contains the genes for the AAV rep and cap proteins and dictates the serotype, and iii) the Ad helper plasmid, which provides the trans adenoviral genes necessary for AAV production. Over a three day period, viral vectors will be produced in bulk in the transfected 293 cells.

The transfected cells will be harvested and the AAV vector preparation purified and concentrated by cesium chloride gradient banding. Approximately 5×10¹³ total viral particles of AAV can be obtained from a 60 plate preparation. This yield will be more than sufficient. Prior to transduction of EPCs, the preparations of the vector will be evaluated for: 1) expression of the gene of interest, and 2) physical particle vector titer. Shown in FIG. 9 is an example validation of TM expression in porcine EPCs which were administered AAV human thrombomodulin vector.

Anti-TM antibodies specific to pigs will be secured. In the meantime, a pig specific bioassay, which quantifies the production of activated protein C(PC) as a function of activity of cellular porcine TM, will be used. To that end, the genetically enhanced EPCs will be incubated with porcine 11a (5 nM, final concentration), added to a mixture of HEPES-buffered saline (pH 7.4, 2 mM CaCl₂) for 5 min at 37° C. This will be followed by the addition of porcine PC (400 nM, final concentration). At various time points, the supernatant will be removed. Free IIa will be inhibited with a mixture of hirudin, antithrombin III, and heparin. The amount of aPC formed will be evaluated using the synthetic substrate spectrozyme PCa (American Diagnostica Inc., Stamford, Conn.) in a microtiter plate reader by the change in absorbance/time at 405 nm. From these studies, it will be possible to determine the amount of TM activity.

Titanium tubes lined with autologous EPCs will be surgically implanted into the inferior vena cava of those pigs from which the EPC were derived. Four groups of 10 pigs in each group will be compared. Specifically, 30 kg to 45 kg Yorkshire Swine will be obtained from a local breeder. Pigs will be maintained at the Duke University vivarium for 4 weeks after their first blood draw for EPC isolation. One group of pigs will undergo implantation of EPC-lined titanium tubes. The second group will undergo implantation of titanium tubes lined with genetically enhanced EPCs. In the third group, uncoated, bare titanium tubes will be implanted. The fourth group will be a control group undergoing sham operations only, without implantation of any device. 48 pigs that had EPCs isolated from their blood will be randomized to 4 groups of 12 pigs each. It is anticipated that at least 85% of animals will survive the operation and post-operative course. Therefore, at least 10 pigs in each group will be available for the entire duration of the study. This number of animals will give the analysis a power of 91% assuming an effect size of 40% in the most important biomarkers to be clinically meaningful (see below).

Animals will be sedated with a combination of Acepromazine Maleate (1.1 mg/kg IM) and Ketamine Hydrochloride (22 mg/kg IM), as well as Isoflurane (0.5-5% inhalation). Immediately before skin incision, blood will be obtained from the femoral vein for biomarker and proteomic analysis. All blood samples during the study will be obtained from the femoral vein. A midline laparotomy will be performed where the abdominal contents will be retracted to the animal's left, exposing the infrarenal vena cava (IVC). A 7 cm segment of vena cava will be isolated and encircled with vessel loops and 100 units/kg of heparin will be intravenously given to the animal. The IVC will be clamped proximally and distally using standard vascular clamps and a 3 cm veinotomy will be made on the anterior surface of the IVC. The PTFE coated titanium implants will be inserted into the IVC and the veinotomy will be sutured closed. Circumferential sutures will then be placed through the IVC wall to the PTFE cuffs both proximally and distally to the implant to ensure that no blood can flow behind the titanium implant. Thus, the blood is only exposed to the inside surface. Flow in the IVC will be reestablished by the removal of the vascular clamps and hemostasis will be ensured. The laparotomy will be closed and the pig will be maintained under anesthesia for 1 hour where a second blood sample will be collected from the contralateral femoral vein. The pig will then be recovered from surgery and returned to its standard vivarum housing.

Peripheral blood will be obtained from the femoral vein at four different time points. All blood samples will be used for biomarker and proteomic analysis. In order to evaluate the biocompatibility of the various titanium tube implants, known markers of thrombosis and inflammation will be quantified as follows: Thrombin Antithrombin Complex (TAT) with Enzygnost TAT micro (Siemens, Newark, Del.), Prothrombin Fragment 1+2 (F₁₂) with Enzygnost F 1+2 (monoclonal) (Siemens, Newark, Del.), D-dimers with Advanced D-Dimer (Siemens, Newark, Del.) and activated Protein C (aPC) with Berichrom Protein C (Siemens, Newark, Del.). All these test kits have been tested in swine and normal porcine reference values are available for comparison (Velik-Salchner et al, Thromb. Res. 117(5):597-602 (2006)). TAT, F₁₂ and D-dimers will be evaluated because these important biomarkers are often used in clinical practice and have been found elevated in patients with mechanical assist devices (Spanier et al, J. Thorac. Cardiovasc. Surg. 116(5):860-869 (1998), Joshi et al, Interact. Cardiovas. Thorac. Surg. 6(3):323-327 (2007), Koster et al, Ann. Thorac. Surg. 70(2):533-537 (2000)). Furthermore, aPC is a very useful marker to measure since one of the pig groups will have thrombomodulin overexpressing EPCs implanted and thrombomodulin activates protein C. The following inflammatory markers will be quantified with porcine specific assays: Interleukin 1β (IL-1β) with Quantikine Porcine IL-1β Immunoassay (R&D Systems, Inc, Minneapolis, Minn.), Interleukin 6 (IL-6) with Quantikine Porcine IL-6 Immunoassay (R&D Systems, Inc, Minneapolis, Minn.), Interleukin 8 (IL-8) with Quantikine Porcine IL-8 Immunoassay (R&D Systems, Inc, Minneapolis, Minn.) and Interleukin 10 (IL10) with Quantikine Porcine IL-10 Immunoassay (R&D Systems, Inc, Minneapolis, Minn.). Again, all of these markers have been measured in patients with implantable circulatory support devices (Clark et al, Eur. Heart J. 22(24):2275-2283 (2001), Miyagawa et al, Surg. Today 38(8):747-750 (2008), Caputo et al, Ann. Thorac. Surg. 74(6):2088-2095; discussion 95-96 (2002), Masai et al, Ann. Thorac. Surg. 73(2):549-555 (2002), Pierce and Larson, Perfusion 20(2):83-90 (2005), Kimball et al, Ann. Thorac. Surg. 85(5):1656-1561 (2008)).

For each biomarker, a Bayesian mixed effects model will be fitted, which incorporates pigs as a random effect to reflect that repeated measurements are being taken on the same pigs over time. The treatment variable will be treated as a fixed effect, with a different time profile permitted in each group. The primary interest is in the distribution of the difference in the treatment means at 7 days and 30 days post-surgery, and whether there are significant changes in the two groups with treated leads and the bare titanium leads. 95% interval estimates will be constructed for the difference in treatment means at these time points; intervals that exclude zero are considered significant at the 0.05 level. The Bayesian approach will allow incorporation of prior information for expected means and variability of each assay under normal control conditions (pre-surgery) based on published literature (Velik-Salchner et al, Thromb. Res. 117(5):597-602 (2006)) and assay package inserts. The inclusion of such prior information can increase the power to detect changes over a standard analysis. Because ELISA-data are often subject to outliers, log transformations of the data will be explored or a heavy tailed error distribution will be uses to provide robustness to potential outliers. Lastly, because these markers may be up/down regulated together, the single biomarker model will be extended to a multivariate model that considers all markers simultaneously and provides an automatic multiplicity adjustment to address false discoverers that may arise from multiple hypothesis testing. For statistical analysis, the R software package and WinBUGS will be used.

It is anticipated that the prothrombotic markers TAT and F₁₂, and the proinflammatory cytokines IL-1β, IL-6 and IL-8 will be upregulated immediately after surgery, but will have returned to their baseline at the 7^{th} postoperative day in the sham surgery group. It is expected that the uncoated Ti implants will persistently increase the prothrombotic state, indicated by an increase in TAT and F₁₂ and possibly a concomitant increase in fibrinolysis as a counterregulatory mechanism, indicated by an increase in D-dimer. Furthermore, the proinflammatory cytokines IL-1β, IL-6 and IL-8 may not return to baseline after 7 or even 30 days.

Since the bare titanium tubes have a fairly large diameter, it is believed that it is unlikely that they will become totally occluded; however, this cannot be stated with certainty given the slightly hypercoagulable state of porcine blood at baseline. It is anticipated that a relative decrease in prothrombotic and proinflammatory markers and possibly an increase in the anti-inflammatory cytokine IL-10 will be found in those pigs which received titanium implants lined with their own EPCs. If so, this difference should be even more pronounced in the pigs with genetically enhanced EPCs. In these animals, an increase in the antithrombotic and anti-inflammatory serine protease activated Protein C is also expected.

At the end of the study, pigs will be anesthetized and the last blood sample obtained. Following, the pigs will be euthanized by intravenous administration of Pentothal, 100 mg/kg. The titanium tubes will be carefully explanted and grossly inspected for clot formation. Then the titanium tubes will be disassembled into their 4 identical longitudinal sections. Two sections will be evaluated with SEM and two sections with fluorescent microscopy. In order to assess thrombogenicity of the titanium samples, 25 areas per sample will be scored blindly by two reviewers for the total number of adherent platelets per image. EPCs were labeled with Qtracker (Molecular Probes, Invitrogen, Eugene, Oreg.) prior to their seeding onto titanium and are now further stained with DAPI. This way, it will be possible to distinguish the original cells on the titanium surface from possible additional ‘fallout’ cells derived from the animal after implantation. Confocal microscopy will be performed using a Zeiss LSM 510 upright laser scanning confocal microscope. Z-sections will be taken and projection images used for analysis to ensure all cells present on the curved surfaces will be detected. Images will be imported into MetaMorph software for cell counting and analysis. Twenty five areas per slide will be scored to determine cell density. Platelet concentrations in the implant groups will be compared using one-way analysis of variance or deviance. Because concentration measurements often have skewed distributions, a determination will be made as to whether log-normal or gamma distributions are more appropriate for these data. A similar modeling approach will be used for the cell density data.

Proteomic analysis will be performed on all peripheral blood samples, comparing the four different groups and time points within each animal in order to determine the differential expression levels of known proteins associated with coagulation and inflammation. The search for specific biomarkers will use a targeted proteomics approach, in which a subset of proteins will be targeted in order to characterize their differential expression as both a function of treatment (EPC Ti implants, genetically-enhanced EPC Ti, uncoated titanium implants, sham group) and time (blood samples collected immediately before surgery, immediately after surgery, 7 days after surgery, and 30 days after surgery). The proteins selected (Table 1) are derived from a general group of known, well-characterized proteins that have been found to be critical in hemostatic and inflammatory pathways in vivo. It is believed that the expression profiles of these proteins will provide a good representation of how the different implants in the experimental groups will interact with blood in an animal model. Moreover, the proposed list of proteins for analysis will provide a multidimensional perspective of biocompatibility by focusing on groups of proteins that are associated with the coagulation and fibrinolytic pathways, as well as the inflammatory system and complement cascade.

TABLE 1
Selected Proteins

| | |
|---|---|
| Procoagulant Proteins | Fibrinolytic Proteins |
| Prothrombin | Tissue Plasminogen Activator (tPA) |
| Thrombin | Plasminogen |
| Prothrombin Fragment 1 + 2 (F₁₂) | Plasmin |
| Thrombin Antithrombin | D-dimer |
| Complex (TAT) | Antifibrinolytic Proteins |
| Factor IV Activation Peptide | Alpha 2 Antiplasmin |
| Factor V | Thrombin Activatable |
| Factor Va | Fibrinolysis Inhibitor (TAFI) |
| Factor VII | Complement Activators |
| Factor VIIa | C3 |
| Factor IX | C3a |
| Factor IXa | C5 |
| Factor X | C5a |
| Factor Xa | Complement Inhibitors |
| Factor X Activation Peptide | C1 Inhibitor |
| Factor XI | Decay Accelerating Factor (DAF) |
| Factor XII | Anti-inflammatory Mediator |
| Factor XIII | Interleukin 10 (IL-10) |
| Fibrinogen | Proinflammatory Mediators |
| Protein Z | C Reactive Protein (CRP) |
| Anticoagulation Proteins | Interleukin 1β (IL-1β) |
| Protein C | Interleukin 6 (IL-6) |
| Activated Protein C (aPC) | Interleukin (IL-8) |
| Protein S | |
| Tissue Factor Protein | |
| Antithrombin (ATIII) | |
| Alpha 2 Macroglobulin | |

The differential expression of this particular set of proteins will be analyzed using analytical protein microarrays (Hall et al, Mech. Ageing Dev. 128(1):161-167 (2007)). Compared to the traditional and well-established method of quantifying known biomarkers with enzyme-linked immunosorbent assays (ELISA's), this novel proteomics approach offers the advantage of being high-throughput and allowing the analysis of a large number of proteins at once. Antibodies for all the proteins presented in Table 1 are commercially available. However, at this time, only a fraction have been tested and found to react with specific swine antigens. For those proteins where pig-specific antibodies are not available, antibodies against proteins from other species, e.g., human, will be used and their cross-reactivity with pig proteins tested using Western blots. Protein chips will be created using a Contact Pin Microarrayer (OmniGrid 100, Genomic Solutions, Ann Arbor, Mich.) at the UNC Systems Proteomics Core facility (http://www:med.unc.edu/sysprot/). Each antibody will be printed in eight serial dilution values. Dilution ranges will be determined after preliminary analysis to determine specific levels of expression for each of these proteins. Most protein chips thus will consist of 42×8 spots, and each chip will be created in triplicate. Antibodies will be spotted in a diameter of 300 μm and with a volume of 5-20 nanoliters, depending on antibody concentration and range of detection. The blood will be depleted of the most abundant proteins (albumin and immunoglobulins) using an Albumin-IgG Depletion Kit (GE-Healthcare, Piscataway, N.J.). Briefly, protein samples are first cleaned with the 2D-clean up kit, then protein lysates are resuspended in Ripa buffer (Sigma, St. Louis, Mo.) to reach a critical concentration of 5.0-10.0 mg/mL. A 15 μL aliquot of the lysate is added to the depletion resin, and albumin and IgGs are removed after a short incubation followed by separation on a bench top centrifuge. Depleted proteins will be labeled with CyDyes (Cy2, Cy3, or Cy5) (GE-Healthcare, Piscataway, N.J.), and mixed with the antibodies previously immobilized on the modified glass slides. The slides will be imaged with the Typhoon 9410 (GE-Healthcare, Piscataway, N.J.) laser scanner. This instrument is designed to determine the amounts of proteins labeled with Cy dyes. The serial dilutions will be used to determine the binding affinity between the antibodies and the target proteins (antibody titer). Comparing the fluorescence of protein lysates labeled with the three different colors will allow the determination of expression changes between control and treated samples.

Array-Pro Analyzer software (Li-cor Biosciences, Lincoln, Nebr.) will be used that includes statistical packages. The methodology described above is expected to enable detection of changes in protein expression down to the nanogram level. Mixed-effects models will be fitted to the log intensities obtained from the protein arrays to determine which proteins are differentially expressed between the treatment groups and over time. Based on unpublished results from the analysis of blood samples from pediatric patients undergoing cardiopulmonary bypass, it is anticipated that approximately 10-15 proteins out of the 42 selected will change consistently with the study groups (EPC Ti implants, genetically enhanced EPC titanium, uncoated titanium implants and sham group) at the various time points. It is anticipated that a subset of these proteins will be found that reflect the effects of EPC seeding, and genetically enhanced EPCs, versus bare titanium implants. Consistency between the results found in the biomarker analysis and the analytical protein microarray results is expected. The knowledge of which specific proteins are over- and under-expressed—and to what degree—will provide the information needed to extend the understanding of the biocompatibility of blood contacting surfaces. Finally, the expected protein panel may give rise to novel biomarkers of biocompatibility of blood-contacting surfaces.

Although the protein array approach is expected to be sufficient to determine the relative expression levels of all of the target proteins, if differences in the target protein expression are not detected, an immunoprecipitation approach will be used followed by quantitative protein determination and iTRAQ (isobaric tag for relative and absolute quantitation) labeling. Using this mass-spectrometry-based stable-isotope labeling approach, in particular, the Applied Biosystems (AB) iTRAQ 8-plex reagent, it will be possible to compare the protein expression levels of the target proteins in up to eight different time points or treatment in a single experiment. Briefly, proteins will be immunoprecipitated with specific antibodies from each treatment, and then will be digested with trypsin, and labeled with one of the eight mass-balanced deriving reagents. The labeled digests from the samples will be combined, and this mixture will then be analyzed by offline LC followed by matrix-assisted laser desorption ionization tandem mass spectrometry (MALDI-MS/MS) or on-line LC/electrospray ionization (ESI)/MS/MS. Proteins will be identified from the peptide molecular weights and the MS/MS sequence data, while the expression ratios will be determined by the relative abundances of the reporter ions in the MS/MS spectrum (m/z 113,114,115,116,117,118,119,121). The mass spectrometric analysis will be done on either an AB 4800 BioITRAQ System (for LC-MALDI), or on a ThermoFisher Orbitrap-FT (LC-ESI/MS/MS) equipped with HCD to enhance the relative abundances of the reporter ions. The software packages used for this analysis (Protein Pilot, Proteome Discoverer, and/or Mascot) all provide statistical confidence levels of both the protein identification and the expression ratios. Unlike normal “shotgun” iTRAQ experiments, “targeted” iTRAQ experiments will be used, focused on selected sensitive iTRAQ-labeled peptides from the targeted proteins. It is expected that the number of functional proteins that will reflect functional response to treatments will be between 10 and 15. Because iTRAQ tags are mass-balanced, all 8 forms of the peptide will have the same peptide molecular weight. This will facilitate development of a method based on an “include” list, which will contain the appropriate mass-to-charge ratios (m/z) for 3 peptides from each target protein. Since the target proteins are known, the target peptides will be selected by a preliminary analysis (by LC-MALDI/MS/MS and LC/ESI-MS/MS) of an iTRAQ-labeled tryptic digest of a mixture of commercially-available standard proteins. The selection criteria will include the peptide sensitivities, the “uniqueness” of the peptide to the target protein, and retention-time information.

### Example 2

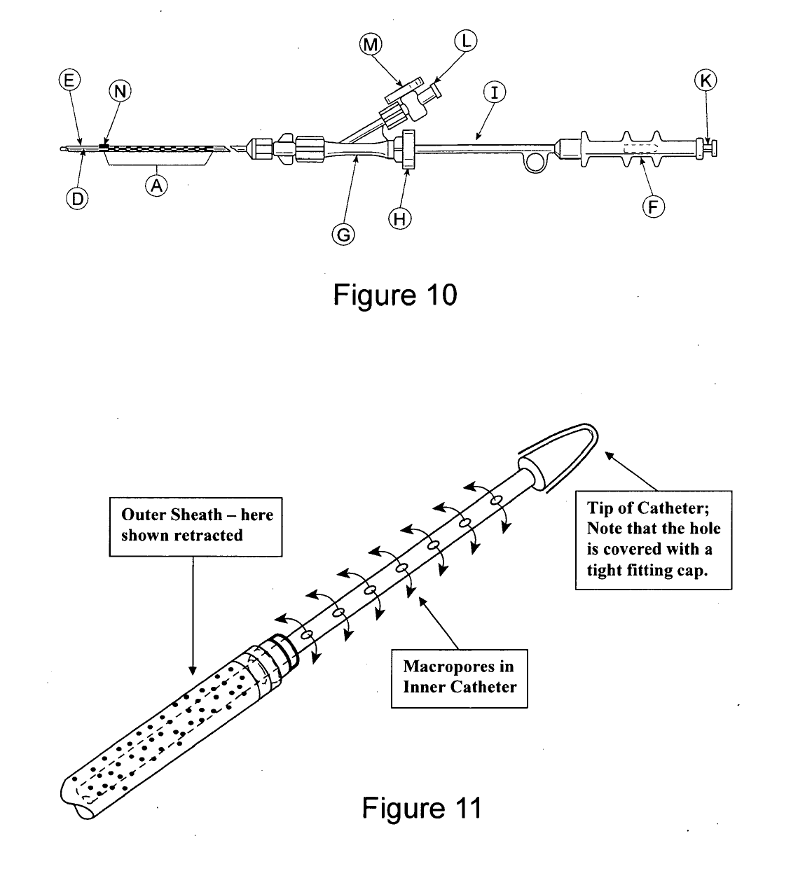

Stent delivery systems to which the present cell (e.g., EPC)-coating methodology can be applied include, for example, the Fluency stent delivery system by Bard, and the Protégé stent delivery system by ev3. Shown in FIG. 10 as an example is the Fluency delivery system by Bard for stents. This principle design is very similar across all the different manufacturers of stents, including, but not limited to, coronary, biliary, vascular and tracheobronchial stents.

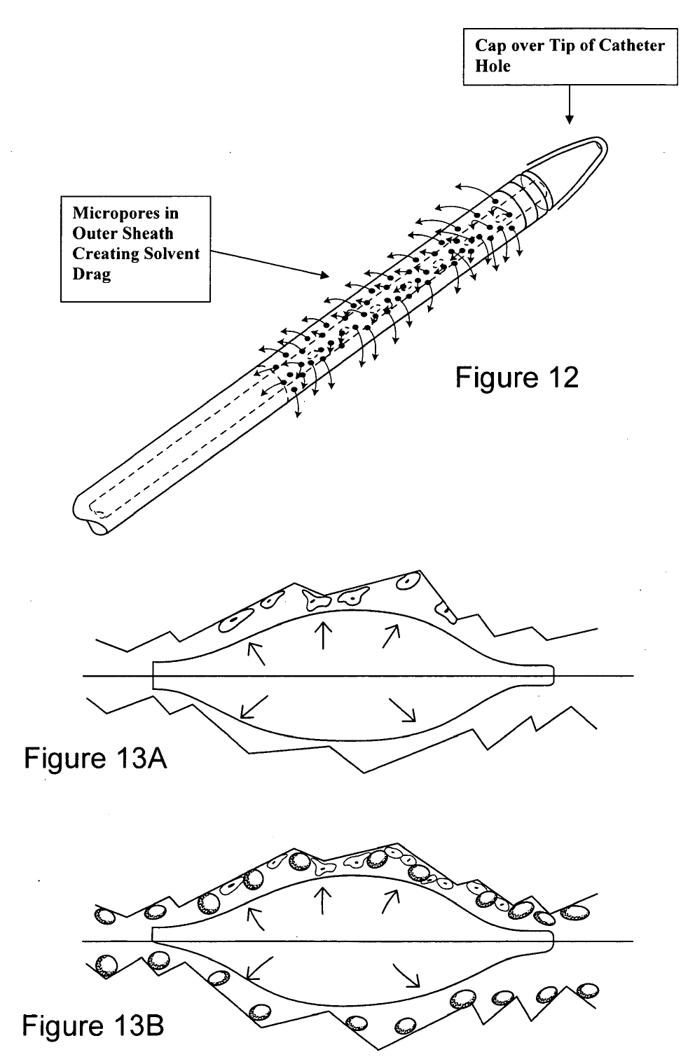

As shown in FIG. 11 a cell-containing solution can be introduced through the distal port (K) of the delivery system (see, for example in FIG. 10) into the inner catheter sheath. The inner catheter can be modified with macropores (about 1 mm diameter) and the hole at the tip of the catheter can be capped such that the cell solution is forced out into the space between the inner catheter and the inside surface of the mounted stent (e.g., a Nitinol stent). The stent is not shown in FIG. 11 but lies on the inner catheter and is constrained by the outer sheath of the catheter. As the cell solution is forced into the space between the inner catheter and the inside surface of the stent, a solvent drag is generated by modifying the outer sheath with micropores (see FIG. 12) so that the solute escapes circumferentially through the micropores and the cells (e.g., EPCs) are dragged onto the inside surface of the self-expanding stent (Nitinol). Once the stent is seeded, the cap is removed and the delivery system can be used in the standard fashion by inserting it into a vessel over a guidewire. The guidewire is passed across the stenotic vessel and serves as the ‘Monorail’ as it guides the stent delivery system into the vessel with the wire sliding inside the inner catheter.

An alternative and preferred approach, which is described in detail in Example 8, is to directly introduce the cell solution into the space between the inner catheter sheath and stent by utilizing a side port (FIG. 10 (L)) of the stent delivery system. A specific example for such a delivery system is the Cordis PRECISE Nitinol Stent System (5.5F Precise, catalog code P05040XC) (4×40 mm unconstrained stent), which is modified in Example 8 below, and which allows for access of the space between the stent and inner catheter sheath via the so called Tuohy Borst Y-connection (in this specific design). Introducing the cell solution into this space obviates the necessity to drill holes (macropores) into the inner catheter sheath, as illustrated in FIG. 11.

Nitinol stents are self-expanding due to their unique ‘memory’ characteristic. Therefore, it is not essential to ‘balloon-expand’ the stent as is the case for stainless steel stents. However, under certain circumstances, self expanding stents (e.g., Nitinol stents) are balloon expanded in order to further open the stent and adapt it better to the contour of the vessel. In order to prevent damage of the cells during balloon-expansion, a modified stent design can be employed.

The modified stent design is applicable to Nitinol or other self-expanding titanium alloy stents. These design principles are applicable to other stents as well, e.g. pure 316L stainless steel stents, such as Cypher stents by Cordis Cardiology, Johnson and Johnson Company, Bridgewater, N.J.

In accordance with the modified stent design, the stent is sintered with titanium microspheres that can provide for ‘safe grooves’ and protect seeded cells (e.g., EPCs) from being damaged during balloon-expansion (see FIG. 13). This allows for ballooning stents with minimal damage to the cells. Other methods to protect the cells include, but are not limited to, immobilizing gold microparticles on the surface, which are larger in size than the diameter of the cells (e.g., EPCs), and electron beam lithography, whereby an electron beam is used to selectively remove regions of the resist covered surface, which results in a micropattern that can be transferred into the metal surface and can provide ‘safe grooves’ for the cells. Alternatively, the stent struts can be modified in such a fashion that they are endowed with small depressions on their inner surface that can protect the cells. These grooves can be generated using a variety of methods, including etching, grinding, mechanical scratching, burning, pitting corrosion by immersion in FeCl₃ at high temperature, electrochemical pitting—application of an anodic potential to the metal in a chloride solution, electrical discharge machining (EDM) and laser ablation. Laser ablation, as well as EDM are the preferred techniques among the latter methods. In EDM, an electrical current discharges between an electrode or wire and the stent metal and the respective size of the electrode or wire determines the diameter of the resulting groove in the surface and should be in the range of several 100 μm.

### Example 3

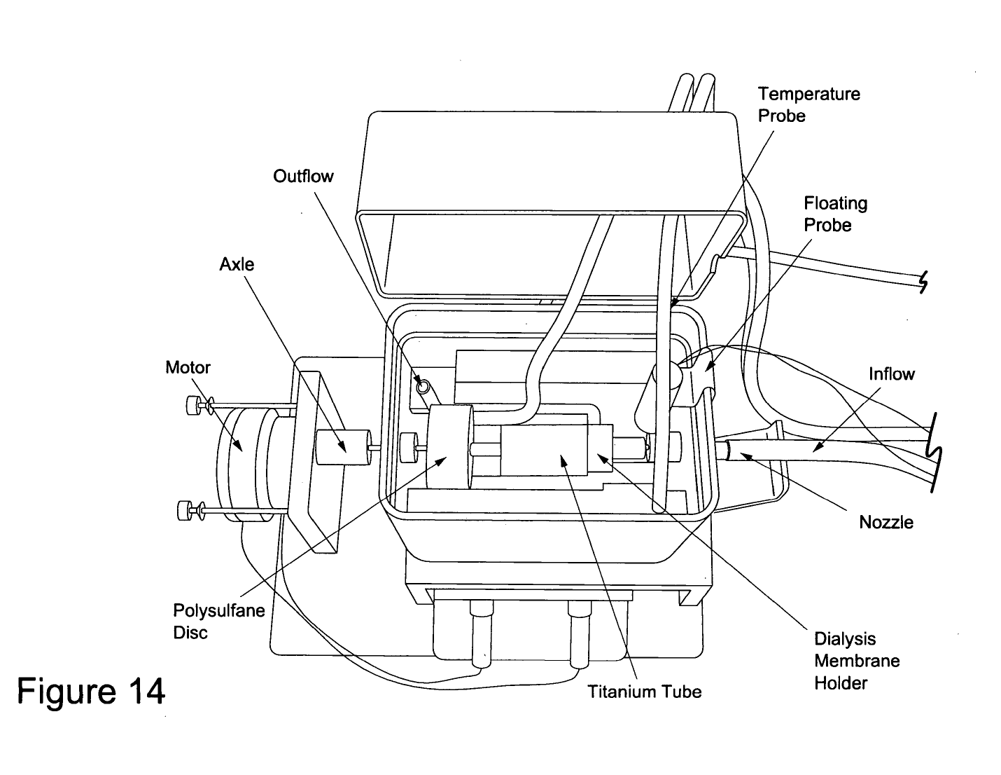

In order to seed EPCs onto the inside surface of titanium tubes, a special seeding device/bioreactor was engineered, which serves to demonstrate how a tube, pump or any other hollow device can be lined with a confluent layer of cells on its inside surface. The seeding device/bioreactor rotates the titanium tube inside a seeding chamber (modified pipet box) filled with cell media (see FIG. 14). The motor (Synchronous Timing Motor, Herbach and Rademan, Morestown, N.J.) is connected via a stainless steel axle through a red rubber stopper to a polysulfone disc. Stainless steel prongs allow for securing the Ti tube, which was loaded into a piece of autoclavable silastic tubing to the rotating polysulfone disc. In order to keep the EPC solution inside the Ti tube, the ends of the silastic tubing were plugged with round dialysis membranes (Spectra/Por Biotech Cellulose Ester Dialysis Membrane, MWCO 1×10⁶, Spectrum Laboratories, Rancho Dominguez, Calif.), which are secured inside polysulfone bulkhead fittings screwed against aluminum inserts covered with O-rings. The entire seeding chamber is filled with cell media so that the rotating titanium tube is entirely submerged during the cell seeding process. The dialysis membranes on either end of the titanium tube allow for gas and media exchange. The seeding chamber is also equipped with a sterilizable floating probe which triggers an alarm, should the media level ever drop during the cell seeding process, e.g. due to a leak. Further, the temperature is monitored via a sterile temperature probe (Mon-a-therm, Tyco Healthcare, Pleasanton, Calif.). The entire chamber is assembled under a laminar flow hood (sterilGARD, Sanford, Main) and then placed into a water jacketed incubator at 37° C. and 5% CO₂. The seeding chamber top is covered with semi-permeable membrane (AeraSeal, EXCEL Scientific, Wrightwood, Calif.), such that gas exchange can occur inside the incubator but no bacterial contamination is possible.

The optimal rotation speed during the seeding process varies in a linear relationship with the diameter of the tube, pump or any other hollow device to be seeded. Specifically, for the dimensions of the titanium tube described above (12 mm diameter), the optimal seeding conditions have been determined at a speed of 10 rotations per hour (rph) with a seeding density of approximately 0.6−2×10⁶ cells/ml, preferably, approximately 1×10⁶ cells/ml, for a duration of 2 hours (also see rationale for cell settling time based upon equation vₛ=(2/9)×(R²/μ)×(ρ_{c}−ρₘ)_{g} described in more detail above). After that, the polysulfone caps on either end of the silastic tubing was disconnected and is the device is perfused through a nozzle opposite one end of the tube (inflow) with an approximate shear stress of 0.5-1 dynes/cm² for a duration of 55 hours, all the while the titanium tube continues to rotate at 10 rph. The outflow returns the media to a pump (Masterflex, Cole-Parmer, Vernon Hills, Ill.) placed outside the incubator. These conditions were determined to be ideal in order to reliably generate a fully confluent lining of EPCs inside these tubes (see FIGS. 8B, 8C, 8D for surfaces generated with this device).

MCADs can be coated with cells (e.g., EPCs) as described below in detail with reference to the HeartMateIII. This MCAD is used for purposes of exemplification only. The principles provided below are applicable to cell-coating any LVAD or artificial heart, independent of manufacturer.

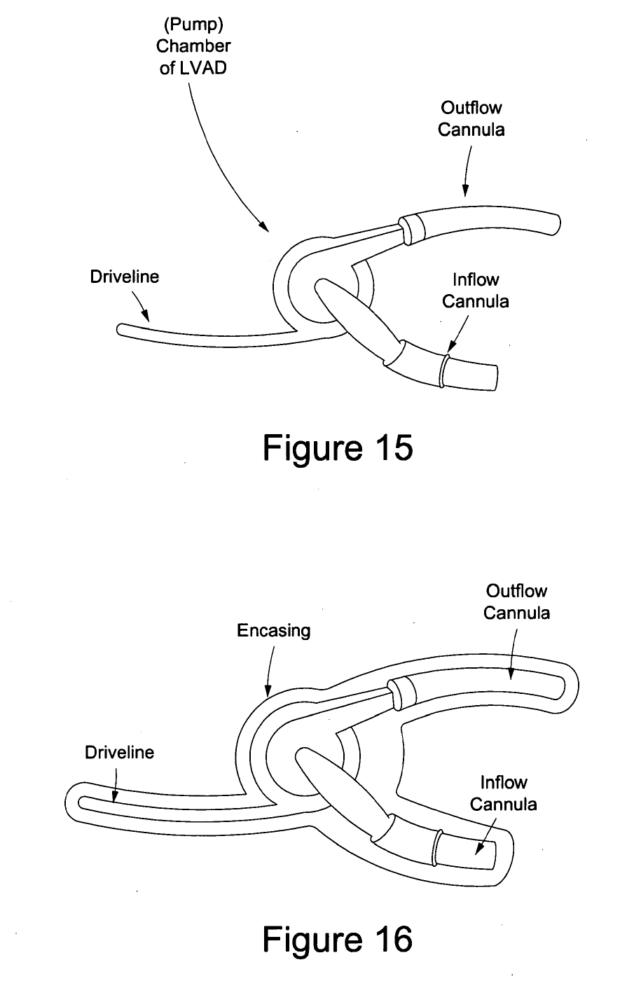

FIG. 15 is a schematic of the main components of the LVAD. Shown are the driveline, which houses the wires that connect the inside motor/pump to the outside environment. When the LVAD is fully implanted, the driveline passes through the patient's skin such that it can be connected to a portable battery or the wall power. Also shown are the inflow and outflow cannulae. The inflow cannula is connected to the heart and allows for blood to flow into the MCAD. The outflow cannula connects the MCAD to the aorta and therefore allows for the blood to be pumped into the circulation.

As shown in FIG. 16, a hard shell “encasing” fully surrounds the entire LVAD. This encasing is molded specifically to the LVAD that is to be cell-coated. The design depicted allows for simultaneous cell seeding of the outside of the driveline (and/or other parts) with a different (non-EPC) cell type (e.g. fibroblasts). This has the advantage of allowing faster tissue in-growth, thereby decreasing the risk of driveline infection. The encasing provides for a hollow surrounding that can be filled with a cell solution, different from the solution used to fill the inside of the device. Depending on the molding, this different cell solution can be in contact with the driveline, or other parts of the LVAD. It also provides a sterile ‘wrapping’ of the MCAD. This encasing can be transparent plastic that can be discarded after opening and removal of the cell (e.g., EPC)-coated LVAD.

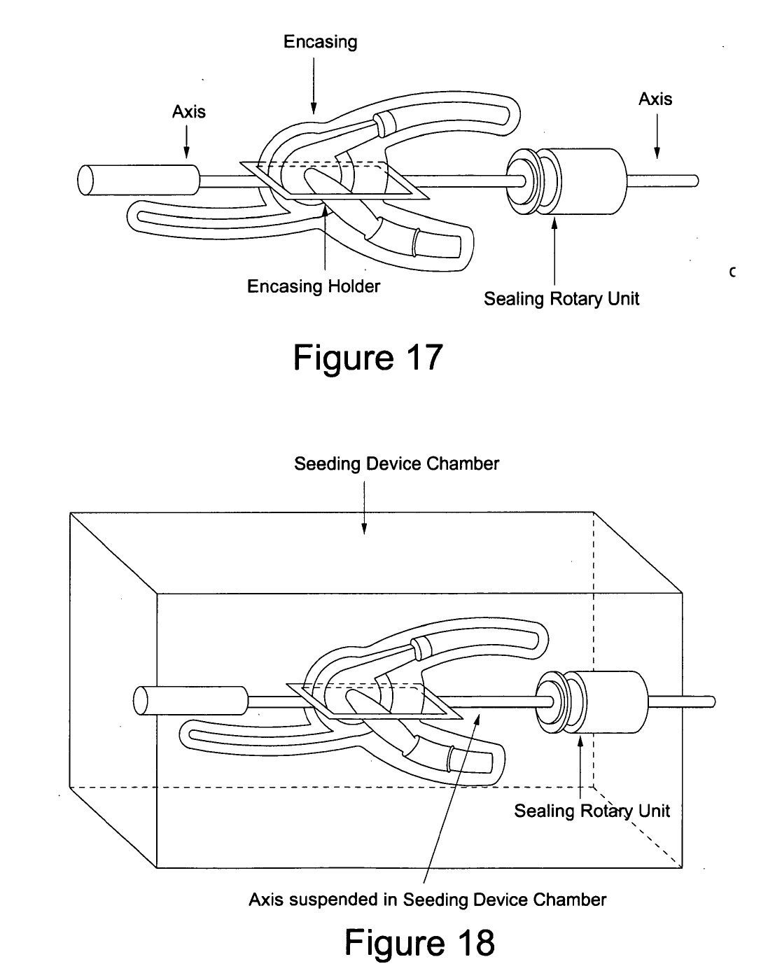

FIG. 17 shows how the encasing holding an LVAD can be turned on its axis. A holder in the middle of the axis allows attachment of the encasing to the axis and it can be opened when the encasing is to be removed and the LVAD is to be implanted. On the right side of the axis, a dynamic sealing rotary unit is depicted. This rotary unit is described in greater detail below. Briefly, it allows for its inner cylinder to rotate together with the axis, but has a fixed outer shell. The left side of the axis is hollow and allows for placement of a rotary electric connector in its inside (see below).

FIG. 18 shows an encased LVAD positioned inside a seeding device chamber. The seeding device chamber shown allows for suspension of the axis on either wall of the seeding device chamber and also fixation of the outer shell of the dynamic sealing rotary unit. The seeding device chamber fully encloses the encasing and LVAD with all its parts. It can be sterilized after assembly and is only opened at the time of use. The seeding device chamber can be constructed of a transparent durable material, e.g. Plexiglas.

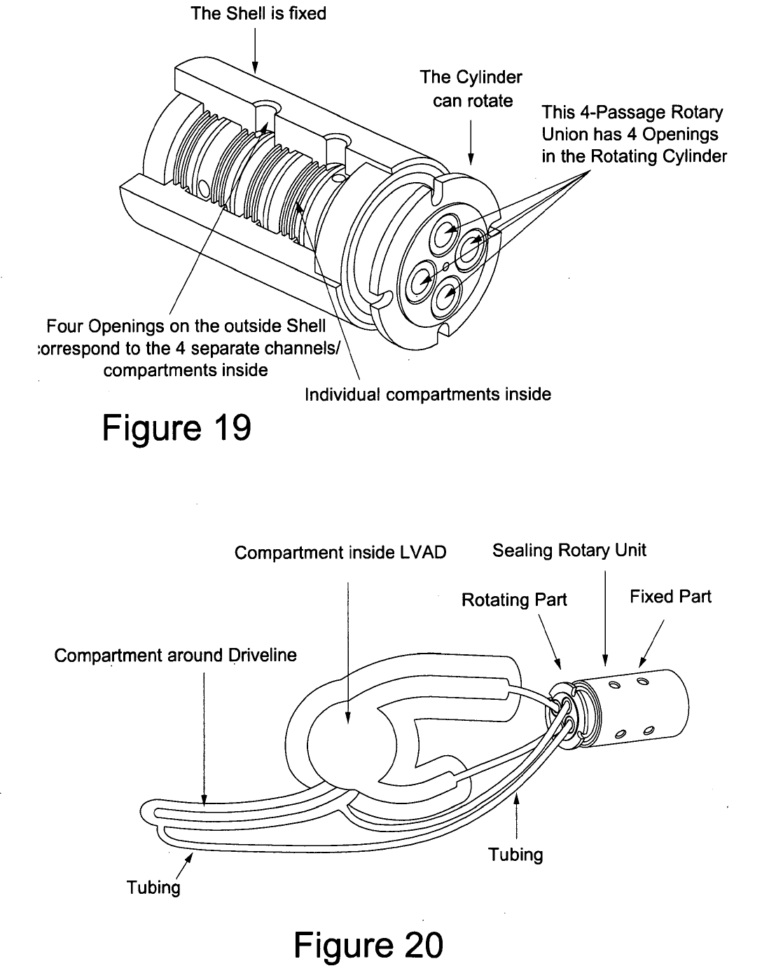

FIG. 19 shows an example of a sealing rotary unit (shown is the GP Series 4-passage rotary unit, Dynamic Sealing Technologies, Andover, Minn.). In this example, four openings in the inner cylinder correspond to four channels inside the rotary unit such that fluid inside the individual channels is keep separate and corresponds to the individual openings in the outer mantle. The purpose of the sealing rotary unit is to allow for perfusion of the different fluid compartments (inside the LVAD and outside, surrounding the driveline). After the initial EPC seeding period, which can take a few hours, the LVAD can be perfused with media in order to eliminate waste products and to provide the cells with nutrients and oxygen. The four separate openings in the cylinder of the rotary unit allow for 360° rotation while the corresponding four openings in its mantle can be fixed. Two of these channels can be used to perfuse the LVAD, with one being the inflow and the other the outflow. The other two can be used to perfuse the fluid compartment around the driveline.

FIG. 20 shows how the sealing rotary unit can be connected to the LVAD (e.g., with tubing) and how the sealing rotary unit can also be connected to a compartment around the driveline (formed by the encasing). This allows for simultaneous perfusion of different compartments (inside of the LVAD and outside of LVAD, specifically the driveline) with two different cell solutions (e.g., EPCs for the inside and, for example, fibroblasts for the outside). In some LVADs, the driveline can be removed. The driveline can be seeded and perfused in a separate chamber.

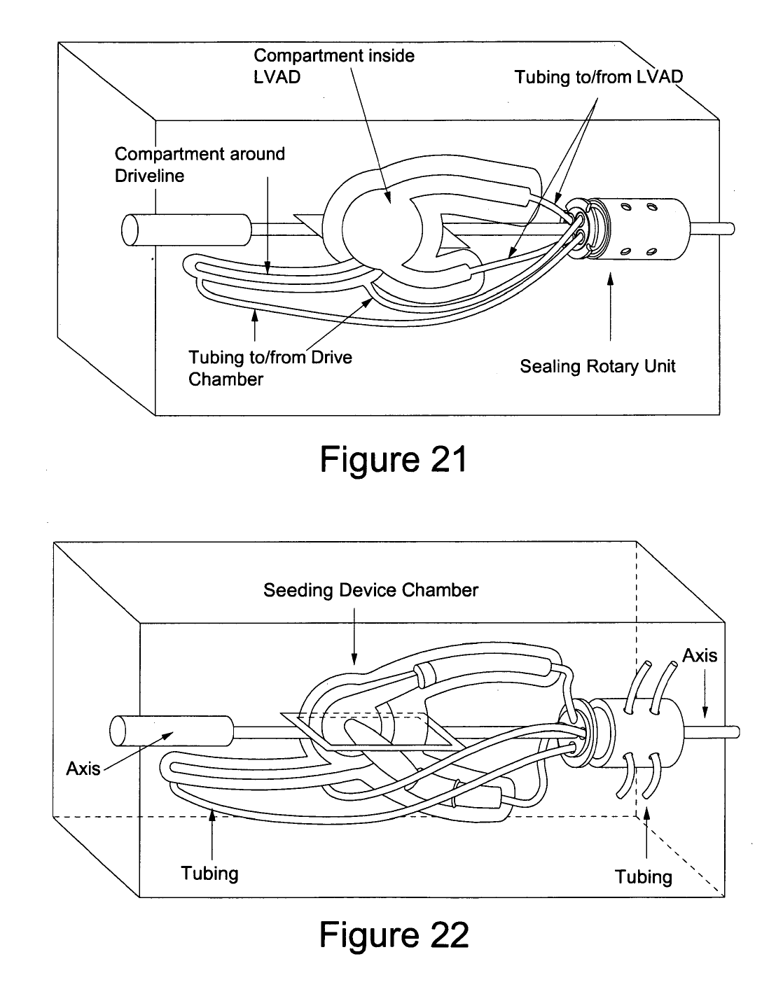

FIG. 21 shows an LVAD positioned inside the seeding device chamber, and depicts how the sealing rotary unit can be connected via tubing to the different compartments.

FIG. 22 shows the LVAD from the outside suspended inside the seeding device chamber with the sealing rotary unit connected via tubing to the different compartments.

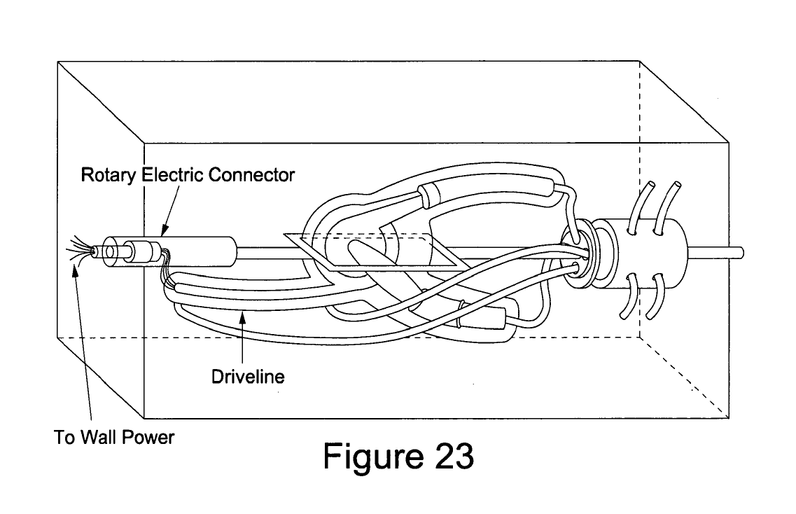

FIG. 23 shows the addition of a rotary electric connector (aka Slip Ring). This rotary electric connector can be connected to the driveline such that the part inside the seeding device chamber rotates with the LVAD and driveline and the outside part of the electrical connection is fixed in position. This design allows for a power connection to the LVAD and enables the LVAD to perfuse itself after seeding. EPCs adherent to the inside surface of the LVAD can sense via their mechanoreceptors exactly those shear stresses that will be experienced after LVAD implantation and can therefore be pre-conditioned ex vivo. The EPCs respond during the pre-conditioning phase by re-arranging their cytoskeleton to optimally withstand the shear forces generated by the specific LVAD. An external pump can also be used to perfuse the LVAD inside chamber.

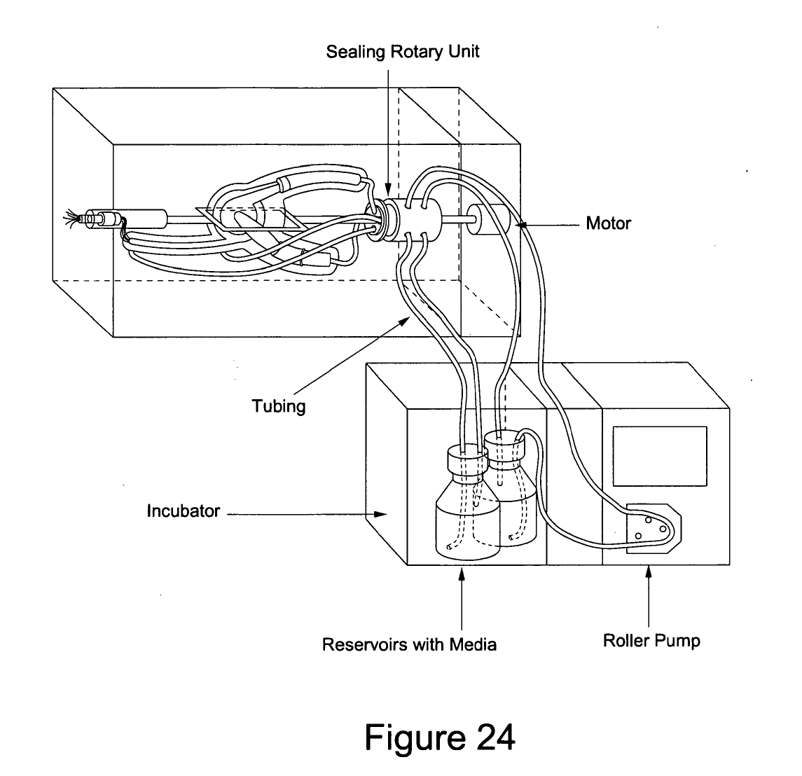

FIG. 24 shows how the axis of the LVAD can be connected to a motor. The tubing leaving the sealing rotary unit in the seeding device chamber can be connected to two reservoirs, for two different cell solutions. An external pump (e.g., a roller pump) is depicted which enables perfusion of the compartment surrounding the driveline. As indicated above, a second pump can be used to perfuse the LVAD inside chamber, if the LVAD does not perfuse itself.

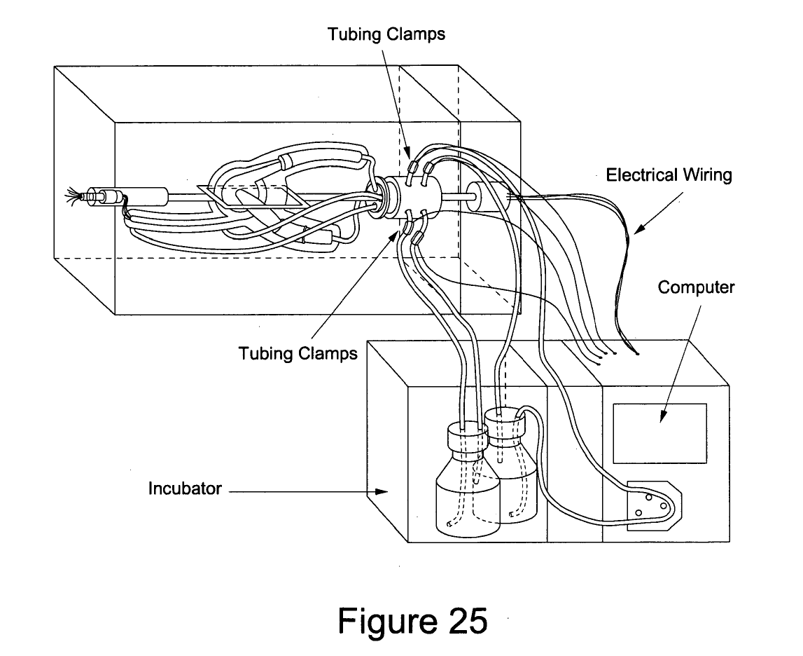

FIG. 25 shows the addition of automated electric tubing clamps which close or open the tubing as it leaves the sealing rotary unit. The electric tubing clamps can be connected to a computer that automatically opens the tubing and starts the pump (as well as LVAD) after the seeding process (mediated by gravity) has finished. The computer can also control the rotation speed of the motor during the initial cell seeding process. The rotation speed can vary within one rotation depending on the geometry of the device. Opening of the tubing can enable perfusion of the cell seeded compartments in order to exchange nutrients, waste products and provide oxygen for the cells. The two reservoirs can be housed in an incubator which regulates the partial pressure of CO₂ and keeps the media at 37° C. temperature.

While the self-contained seeding device/bioreactor has been described above in detail in connection with the seeding of an LVAD, this method of rotating the to be seeded device and perfusing it with media or other solution (at the same time or immediately after cell seeding) to nourish the cells and condition them to the flow after cell adhesion has been achieved (all the while keeping it sterile and self-contained), can be used to seed hollow or cavernous structures/devices other than LVADs. Whereas such a self-contained seeding device and/or bioreactor can be used to seed EPCs onto LVAD surfaces, and other surfaces, and also enable the seeded EPCs to grow into a fully confluent monolayer, a preferred approach is to seed the respective surface quickly, e.g. within minutes on the operating room table or catheterization laboratory, immediately before implantation, and not wait until a fully confluent monolayer has been established. To this end, the EPCs can be seeded as described above, with a self-contained seeding device and/or bioreactor, taking advantage of the gravity-based seeding process, using a rotating seeding device, and implanting the thus seeded surfaces before they reach confluence. For a specific example of such a quick-seeding process, see Example 7.

These approaches can be used, for example to seed vascular grafts, dialysis grafts, artificial vessels, tissue-engineered implantable hollow devices, artificial intestinal tract, tissue engineered bowel, dental implants, artificial/tissue engineered trachea or parts of the bronchial tree, aortic grafts, heart valves, shunts, bile ducts, pancreatic ducts, lacrimal (tear) ducts, cerebrospinal fluid containing shunts, ducts or tubes necessary for the engineering of artificial organs, stents, implantable tubes, pumps, biosensors, implantable tubing and other implantable hollow tube, structure or device.

### Example 4

In order to increase the rate of EPC spreading on titanium surfaces, which can be of advantage if a large surface (e.g., that of a MCAD), is to be covered with a confluent lining of EPCs, an evaluation was made of the effects of changing the media composition (by using serum starved EPC media) and of pre-coating the surface with extracellular matrix proteins (e.g. fibronectin (FN)). Titanium and titanium-alloys influence the conformation and orientation of matrix molecules, such as FN, and cause them to assume a more compact conformation, as indicated by a smaller ΔD/Δf and larger footprint, and with that a different stereochemical arrangement of the RGD integrin binding sites (Hemmersam et al, J. Colloid Interface Sci. 320:110-116 (2008)). It was found that both serum starvation and FN pre-coating can accelerate EPC spreading during the initial hours after seeding.

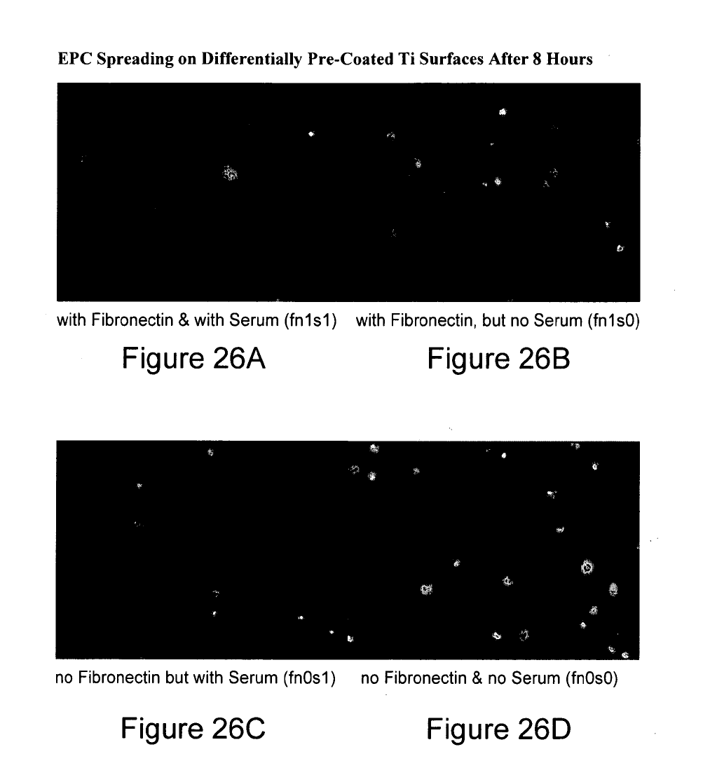

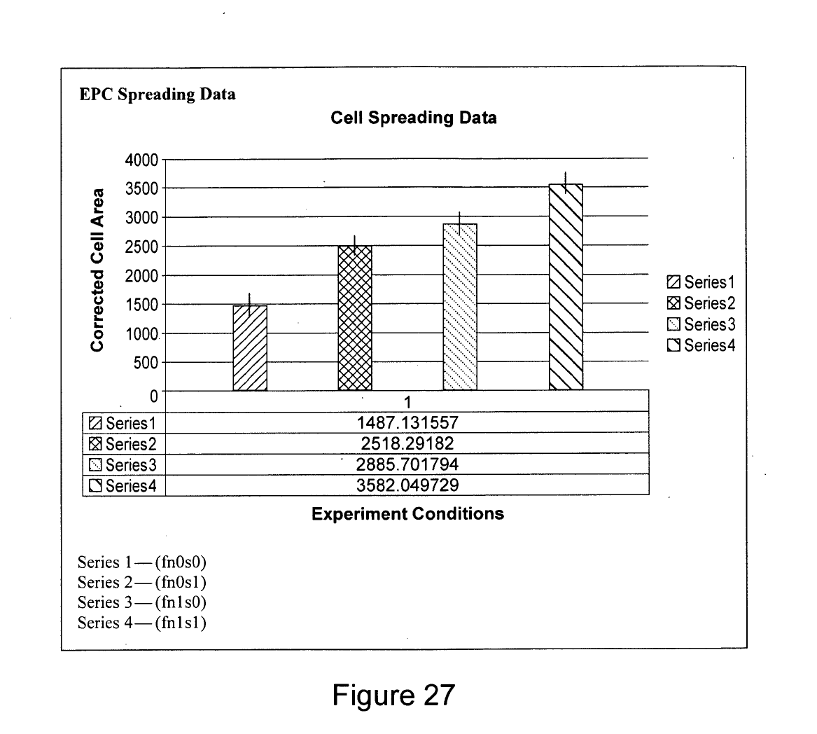

To evaluate the effects of either over a longer time period, a FN pre-coated titanium sample in serum rich media (fn1s1) (FIG. 26A) was compared to a FN pre-coated titanium sample in serum starved media (fn1s0) (FIG. 26B), a bare non-coated titanium sample in serum rich media (fn0s1) (FIG. 26C) and a bare non-coated titanium sample in serum starved media (fn0s0) (FIG. 26D). Pre-coating was achieved by physiosorption of 3 μg human FN (Sigma-Aldrich, St. Louis, Mo.) per cm² surface area in Dulbecco's phosphate buffered saline (DPBS) to titanium for 3 hours at room temperature. EPCs were labeled fluorescently with cell tracker orange and seeded at a density of 6,500 cells/cm². Following incubation for 8 hours, all EPCs were imaged and individual areas measured with Leica software (Leica, Image-Pro Plus Version 6.3, Bethesda, Md.). A total of 1,662 individual EPC areas were quantified in 4 consecutive experiments under the same conditions. Mean surface areas for the four different experimental conditions were calculated. A multiple linear regression model was performed, where experimental condition, experiment number, and the interaction between the two were the predictor variables and surface area was the outcome. The analysis of variance was examined and p<0.05 was accepted as statistically significance. While controlling for experiment number and the interaction, FN pre-coated EPCs had spread significantly faster after 8 hours on titanium as compared to the other experimental conditions (FIG. 27 and Table 2). Data analysis was carried out with SAS version 8.2 (Cary, N.C.).

TABLE 2
Statistical Analysis of EPC Spreading on Different Pre-coatings
A multiple linear regression model was performed, where experimental condition, experiment
number, and the interaction between the two were the predictor variables and surface area was the outcome.

| | Corrected Cell Area LSMEAN | 95% Confidence Limits | |
|---|---|---|---|
| 0 = fn0s0 | 1487 | 1276 | 1699 |
| 1 = fn0s1 | 2518 | 2344 | 2693 |
| 2 = fn1s0 | 2889 | 2686 | 3091 |
| 3 = fn1s1 | 3582 | 3384 | 3780 |

Least Squares Means for Effect (0 = fn0s0, 1 = fn0s1, 2 = fn1s0, 3 = fn1s1)
Pr > |t| for H0: LSMean(i) = LSMean(j)
Dependent Variable: Corrected Cell Area

| i/j | 0 | 1 | 2 | 3 |
|---|---|---|---|---|
| 0 | | <.0001 | <.0001 | <.0001 |
| 1 | <.0001 | | 0.0333 | <.0001 |
| 2 | <.0001 | 0.0333 | | <.0001 |
| 3 | <.0001 | <.0001 | <.0001 | |

| Source | DF | Sum of Squares | Mean Square | F Value | Pr > F |
|---|---|---|---|---|---|
| Model | 15 | 2351932951 | 156795530 | 41.26 | <.0001 |
| Error | 1646 | 6255205381 | 3800246 | | |
| Corrected Total | 1661 | 8607138331 | | | |

### Example 5

An investigation was made of the surface roughness of the titanium samples used in the experiments described above. The best results, in terms of cell spreading and adhesion, were achieved on very smooth surfaces.

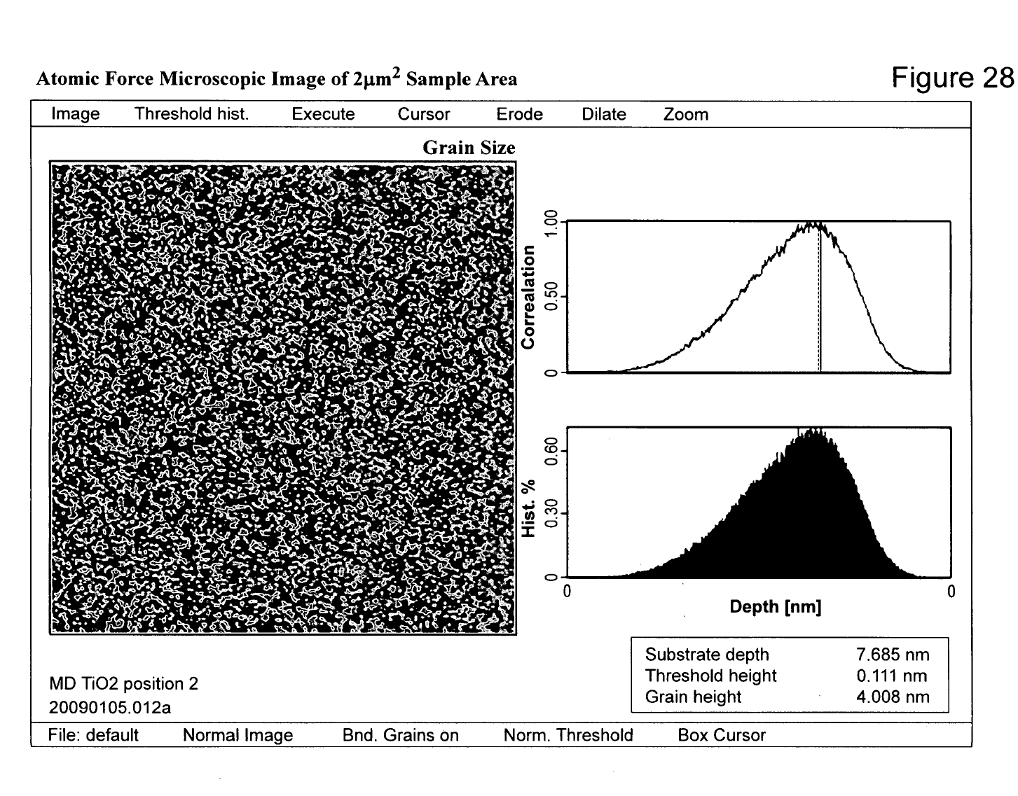

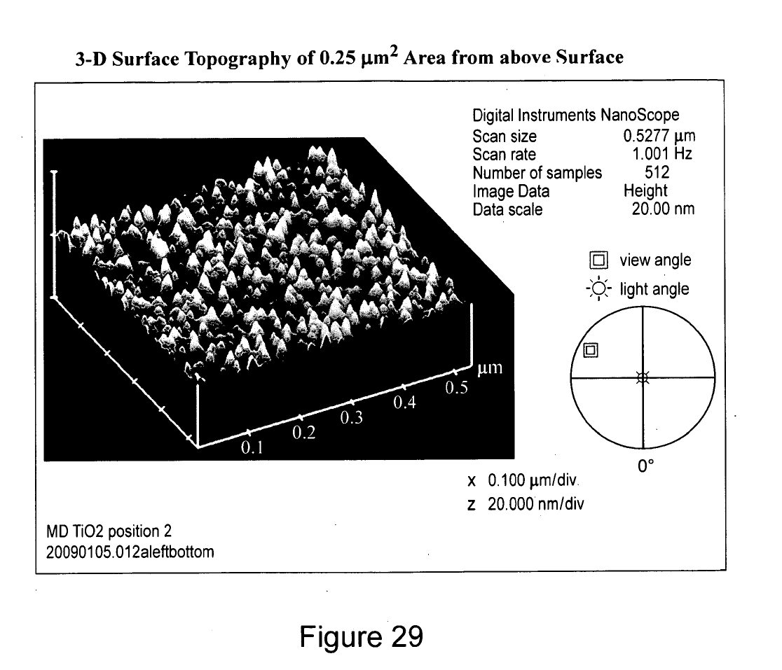

To analyze and quantify the surface roughness/grain size of the titanium samples, they were subjected to AFM measurements. First, the samples were rinsed with SDS, dried under a stream of nitrogen, and then mounted on steel sample disks. AFM topographic images were performed in air and obtained under low applied normal forces (<1 nN), and collected in contact mode using V-shaped silicon nitride cantilevers (Nanoprobe, Veeco, spring constant 0.12 N/m; tip radius 20-60 nm) using a MultiMode atomic force microscope (Digital Instruments, Santa Barbara, Calif.). The average surface roughness measured over a 2 μm² area was found to be approximately 11 nm (FIG. 28). FIG. 29 depicts the three-dimensional image of the surface topography of the titanium samples.

### Example 6

### Experimental Details

Titanium Slide Manufacturing: 100 nm of Ti was deposited on piranha-cleaned glass slides (Fisher Scientific) using a CHA Industries Solution E-beam Evaporation System. The Ti purity was 99.999%, the deposition rate was 2 Å/s, the pressure was 5×10⁻⁶ Ton. Slides were rotated continuously at 20 rotations per minute during the deposition process as previously described (Lin et al, Biomed. Mater. 4(1):015013 (2009)).

X-ray Photoelectron Spectroscopy: The atomic composition of the Ti samples was determined using a Kratos Axis Ultra X-ray Photoelectron Spectroscope and compared to the inner surface of a HeartMate II MCAD adapter, provided by Thoratec as previously described (Achneck et al, Microsc. Res. Tech. 73(1):71-76 (2010)).

Spectra were obtained at 2.0×10⁻⁸ Torr using a monochromated aluminum K-alpha X-ray source at a power of 15 kV and an emission current of 10 mA. Scans were performed over the range of 5-1,200 eV with a step eV of 1, a dwell time of 200 ms and a resolution of 160 eV. The surface composition of the samples was calculated from survey scans using CasaXPS software and utilizing a relative sensitivity function library specific to the Kratos Axis Ultra system.

Atomic Force Microscopy: A MultiMode atomic force microscope (Digital Instruments) was used to assess the surface topography and roughness of our samples (Chen et al, Advanced Materials 21(18):1825-1829 (2009)).

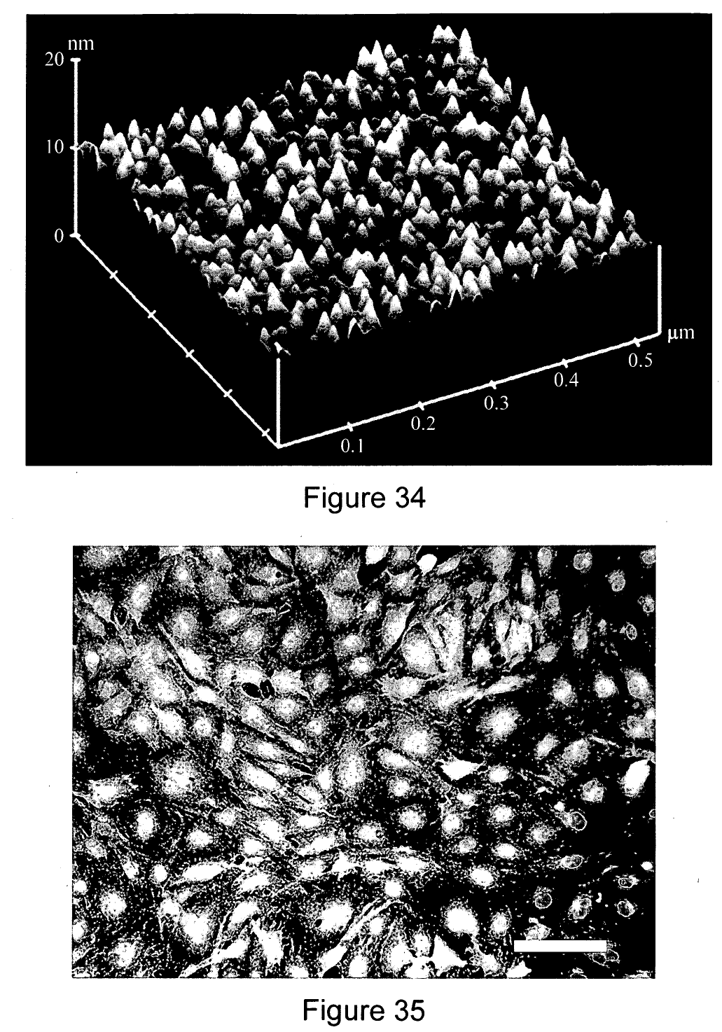

Ti/TiO₂-coated samples were rinsed with 2% sodium duodecyl sulfate solution followed by 30 rinses in deionized water water, then dried under a stream of nitrogen, and imaged (using contact mode) in air with a V-shaped silicon nitride cantilever (Nanoprobe, Veeco, spring constant 0.12 N/m; tip radius 20-60 nm) using a MultiMode atomic force microscope (Digital Instruments). The roughness (R_{q}) was determined with Nanoscope 3 software (see FIG. 34) according to formula (I) (DeGarmo et al, Materials and Processes in Manufacturing (Wiley), 9^{th} Ed, page 223 (2003)),

$${R}_{q} = \sqrt{\frac{1}{n} {\sum }_{i = 1}^{n} {y}_{i}^{2}} \qquad ( I )$$

where n is the number of measurements and yᵢ is the vertical distance from the calculated mean line to the i^{th} measurement.

Contact Angle: The surface wettability was assessed by measuring the water contact angle with a Rame-Hart NRL CA 100-00 goniometer (Lin et al, Biomed. Mater. 4(1):015013 (2009)).

To measure the wettability of the Ti surfaces, five different points were assessed per Ti slide on the surface using the sessile drop method at room temperature in air, with a drop volume of 5 μl using a Rame-Hart NRL CA 100-00 goniometer.

EPC Isolation: Animal care and experimentation were in accordance with the National Institute of Health Guidelines for the Care and Use of Laboratory Animals and were approved by the Duke University Institutional Animal Care and Use Committee. Mononuclear cells were collected via density centrifugation and cultured as previously described (Broxmeuer et al, Methods Enzymol. 419:439-473 (in eng) (2006)). EPC colonies formed after an average of 5-7 days. Cells were used at passages 5-10 for all experiments.

The animals were sedated according to protocol and femoral vein access was obtained. After discarding the first 5 ml of blood, 100-150 ml blood of five 35 kg Yorkshire swine were collected into 25 ml Citrate Phosphate Dextrose containing Cord Blood Collection Bags (Pall). Mononuclear cells (MNCs) were collected via density centrifugation as previously described (Broxmeyer et al, Methods Enzymol. 419:439-473 (in eng) (2006)) and resuspended in EPC medium [EBM-2 base medium+EGM-2 SingleQuots (Lonza/Clonetics) and a total of 50 ml fetal bovine serum (Hyclone)]. MNCs were counted and plated at an average density of 9×10⁶ MNCs per cm² surface area into separate wells of E-well tissue culture plates coated with type-I rat tail collagen (BD Biosciences) at 37° C., 5% CO₂, in a humidified incubator. After 24 hrs of culture, nonadherent cells were removed and EPC medium was added to each well. Medium was changed daily for 7 days and then every other day following. EPC colonies formed after an average of 5-7 days. Cells were used at passages 5-10 for all experiments.

Flow Cytometry: EPCs were evaluated for presence or absence of CD14, CD45 and CD31 with a FACSCalibur flow cytometer (Becton Dickinson) as previously described (Stroncek et al, Tissue Eng. Part A 15(11):3473-3486 (in eng) (2009)).

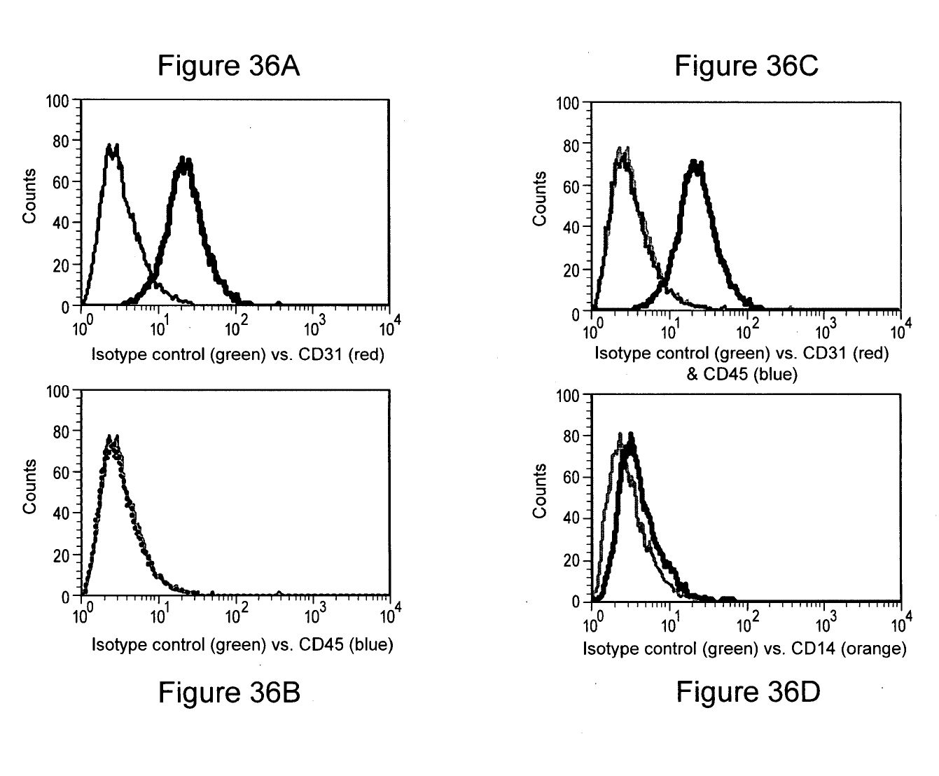

EPCs were suspended at a concentration of 1×10⁶ cells/ml in 1% Bovine Serum Albumin in Phosphate Buffered Saline and labeled with directly conjugated mouse anti-porcine CD14, CD45 and CD31 antibodies (Serotec MCA1746F, MCA1222F, MCA1218F). Respective isotype controls were used (Serotec MCA691F, MCA928F) and fluorescent intensity measured with a FACSCalibur flow cytometer (Becton Dickinson). For each set of samples, the fluorescent intensity of the isotype control was compared with the fluorescent intensity of the test sample using CellQuest software (Becton Dickinson) (see FIG. 36).

Immunohistochemistry/Microscopy: EPCs on Ti were fluorescently labeled and imaged with an upright Leica DMRB microscope as described previously (Stroncek et al, Tissue Eng. Part A 15(11):3473-3486 (in eng) (2009)).

EPC-coated Ti slides for imaging were fixed in 3.7% formaldehyde (Ricca Chemical) and permeabilized with 0.1% Triton X (Sigma-Aldrich). After incubation with anti-porcine CD31 (Antigenix America APG311) and anti-human eNOS (Santa Cruz) diluted 1:50, cells were stained with the secondary antibodies goat anti-rabbit AlexaFlour546 (Invitrogen), and goat anti-mouse AlexaFlour488 (Invitrogen), at 1:500 dilution. Appropriate positive and negative control experiments were performed to rule out non-specific antibody binding to Ti surfaces. Nuclei were counterstained with DAPI (Vector Laboratories). To test for uptake of acetylated Low Density Lipoproteins (LDL), live cells on Ti were incubated (4 hrs, 37° C.) with DiI-labeled acetylated Low Density Lipoproteins (Biomedical Technologies). Fluorescent microscopy was performed with an upright Leica DMRB microscope with a Qimaging Qicam monochrome digital camera and Image Pro Plus software.

Scanning Electron Microscopy: Scanning electron microscope images were obtained with a Philips XL30 ESEM TMP (FEI Company) SEM using standard high vacuum imaging at 25 kV as described previously (Achneck et al, Microsc. Res. Tech. 73(1):71-76 (2010)).

Prior to imaging, samples were fixed in 3.7% formaldehyde, dehydrated in an ethanol series, dried in a Pelco CPD2 critical point dryer (Ted Pella), and coated with Au/Pd (60/40%) in a Hummer 6.2 sputter coater (Anatech). Au/Pd was sputtered from a single alloy source to a film thickness of 6-7 nm.

Redox Assay: EPCs were grown to a confluent monolayer on bare Ti and FN-glass (as control) The alamarBlue redox assay (Invitrogen) (Fields and Lancaster, Am. Biotechnol. Lab. 11(4):48-50 (in eng) (1993)) was added to the respective samples and the change in absorbance with respect to medium measured with a spectrophotometer (Beckman Coulter DU 640). The percent reduction of alamarBlue was determined over the course of 14.5 hrs for samples and controls in 6 separate experiments.

Glass slides were precoated with 3 μg/cm² surface area of bovine fibronectin (Invitrogen) and served as a control to uncoated Ti slides. Equal numbers of EPCs were grown to a confluent monolayer on both surfaces. The alamarBlue redox assay was diluted to 10% in EPC medium and added to EPC-seeded Ti and FN-glass slides (and unseeded Ti slides as control for presence of cells). Its change in absorbance with respect to medium was measured with a spectrophotometer (Beckman Coulter DU 640). The percent reduction of alamarBlue by metabolizing EPCs was calculated according to the manufacturer's instructions as:

$$\% Reduction = \frac{[ 100 \times ( {ɛ}_{OX} ) {λ}_{2} A {λ}_{1} - ( {ɛ}_{OX} ) {λ}_{1} A {λ}_{2} ]}{[ ( {ɛ}_{RED} ) {λ}_{1} {A}^{′} {λ}_{2} - ( {ɛ}_{RED} ) {λ}_{2} {A}^{′} {λ}_{1} ]} \qquad ( II )$$

with (εₒₓ) and (ε_{RED}) the molar extinction coefficient of its oxidized and reduced form, and A, A′ the absorbance of the test well, negative control well, and λ₁=570 nm, λ₂=600 nm. Medium samples were obtained for analysis at the predetermined time points 0 hr, 1.5 hrs, 3.5 hrs, 6.5 hrs, 9.5 hrs and 14.5 hrs. A total of 6 experiments were performed.

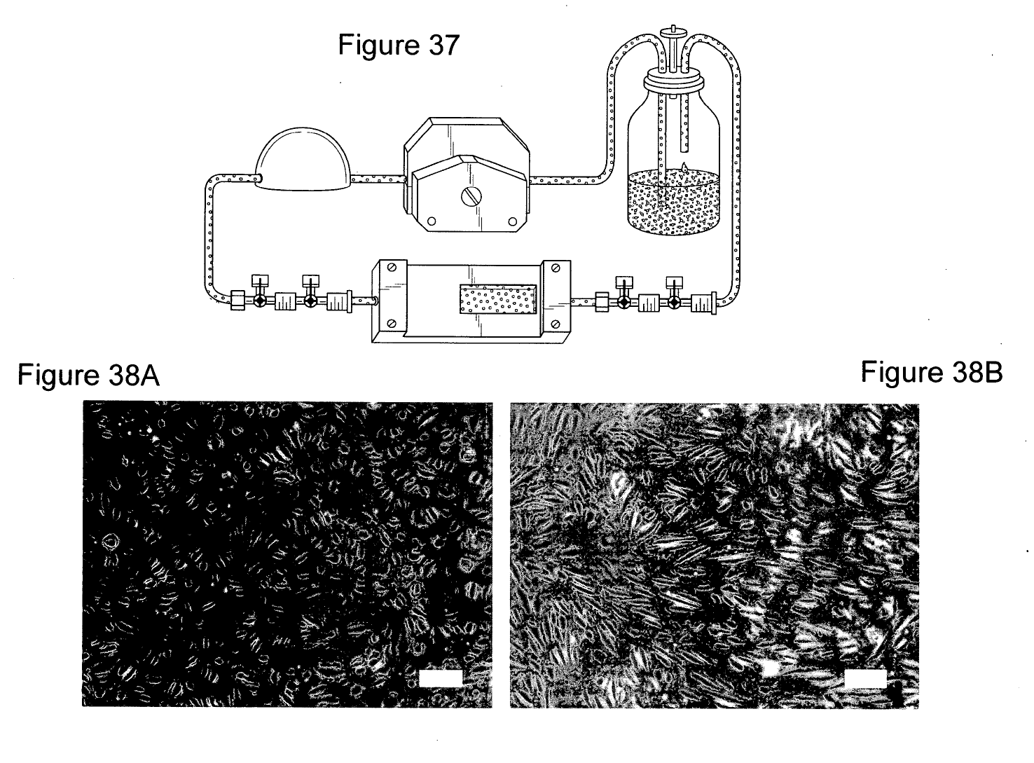

Flow Experiments: To assess EPC adhesion under supraphysiological shear stress, EPCs were fluorescently labeled and seeded onto bare Ti slides at a density of 20,300 cells/cm² and left to settle for 15 min in phosphate buffered saline (PBS) at 37° C., 5% CO₂, in a humidified incubator. Following, the slide was rinsed and transferred into a variable height flow chamber in a flow circuit (see FIG. 37) in full EPC medium (EBM-2 base medium+EGM-2 SingleQuots (Lonza/Clonetics)+10% fetal bovine serum (Hyclone) (Bhat et al, J. Biomed. Mater. Res. 41(3):377-385 (in eng) (1998)).

To evaluate EPC spreading under flow on Ti, EPCs from 3 pigs were seeded on Ti at densities of 200,000 cells/cm² in PBS and left to settle for 15 min before rinsing×3 and exposing to 15 dynes/cm² of flow in full EPC medium.

Further, long term flow experiments were conducted with EPCs incubated×24 hrs prior to flow on bare Ti slides (76,000 cells/cm²) in EPC medium (37° C., 5% CO₂). Glass slides precoated with bovine fibronectin (Invitrogen) at 0.25 μg/cm² served as control.

For all flow experiments, Ti slides were cleaned in 2% sodium duodecyl sulfate solution for 30 min followed by 30 rinses in deionized water and then dried with nitrogen gas. Sterilization was achieved by steam autoclaving at 120° C. for 30 min (AMSCO Scientific Series, Gravity Stage 3).

A laminar flow circuit (total volume 100 ml) was assembled as previously described (Bhat et al, J. Biomed. Mater. Res. 41(3):377-385 (in eng) (1998)) (see FIG. 37). All components of the circuit, except for the pump, were placed into a humidified incubator at 5% CO₂ and 37° C. (NuAire). For static control experiments, Ti slides'(FN-glass slides) were incubated next to the circuit in petri dishes covered by 20 ml EPC medium.

Ti (and control) slides were seeded using Quadri-PERM 4-Compartment Culture Dishes (Greiner Bio One). To assess EPC adhesion under supraphysiological shear stresses as well as spreading under flow conditions, cells were suspended in 5 ml PBS (without calcium/magnesium) per slide and allowed for 15 min settling/adhesion time.

To image EPCs on Ti, cells were fluorescently labeled with Cell Tracker Orange (Invitrogen) at a concentration of 1 mM diluted in DMSO and incubated for 15 min in serum free medium (DMEM/F12, Lonza/Clonetics).

The variable height flow chamber allowed for exposure to different shear stresses along the direction of flow inside the chamber as previously described (Bhat et al, J. Biomed. Mater. Res. 41(3):377-385 (in eng) (1998)). The radiopaque Ti was imaged through the transparent top of the flow chambers with an upright fluorescent microscope (Leica). Shear stress was calculated according to

$$τ = \frac{μ Q}{{wh}^{2}} \qquad ( III )$$

where τ denotes the wall shear stress (dynes/cm²), μ, the medium viscosity at 37° C. (0.01 gcm⁻¹s⁻¹), w is the channel width (1.8 cm), h the channel height (0.025 cm), Q the volumetric flow rate (cm³/s). After flow, images were obtained at the exact same positions as prior to flow through the chamber top. The fraction of adherent cells was calculated as the ratio of adherent cells per position after flow to the number of adherent cells per position before flow. Roundness was calculated as

$$Roundness = \frac{4 \times π \times Area}{{Perimeter}^{2}} \qquad ( IV )$$

For evaluation of EPC adherence under supraphysiological shear stress, 25 images (100× magnification) were obtained in 5 predetermined positions (channel heights) and 500 cells counted per position. EPCs were then exposed to shear stress, ranging from 46 dynes/cm² to 418 dynes/cm². After flow, images were obtained at the same positions as prior. The fraction of adherent cells was calculated in 14 experiments with EPCs isolated from 3 different pigs and 68 different shear stresses.

To evaluate EPC spreading under flow conditions, the cells were exposed to 15 dynes/cm² after seeding and images were obtained at time points 0 hr, 24 hrs and 48 hrs (at 100×). Cell area and roundness were determined for 500 cells per time point using Image J software.

For long term flow experiments, Ti slides were placed in a parallel plate flow chamber and exposed to continuous flow at 15 dynes/cm² or 100 dynes/cm²×48 hrs. With every experiment, a control slide, either Ti or FN-coated glass, was seeded and incubated under static conditions. 15 images (200×) in 24 experiments were obtained along the axis of the slide prior to and after flow. Areas, roundness and angels (relative to the direction of flow) were quantified for 500 cells before and after flow (Image J software) per slide.

NO Quantification: The concentration of the nitric oxide (NO) metabolite nitrite (NO₂⁻) was measured by chemiluminescence with an Ionics/Sievers Nitric Oxide Analyzer (NOA 280, Sievers Instruments, Boulder, Colo.) as described previously (Allen et al, Nitric Oxide 20(4):231-237 (in eng) (2009)).

To quantify NO production by EPCs, the primary oxidation product nitrite was directly measured in 150 μl medium samples collected from the flow circuit reservoir and static control at 0 min, 1 hr, 6 hrs, 12 hrs, 24 hrs, 48 hrs in 24 experiments. Samples were frozen at 80° C. until further analysis. The concentration of the nitric oxide (NO) metabolite nitrite (NO₂⁻) was measured by chemiluminescence with an Ionics/Sievers Nitric Oxide Analyzer (NOA 280, Sievers Instruments, Boulder, Colo.) as described previously (Allen et al, Nitric Oxide 20(4):231-237 (in eng) (2009)). The reductant used for nitrite analysis was potassium iodide in acetic acid (14.5 M acetic acid and 0.05 M KI), which has the reduction potential to convert nitrite to NO but is insufficient to reduce any higher oxides of nitrogen such as nitrate and thus is relatively specific for nitrite. The total amount nitrite produced was calculated as the product of concentration produced and the total volume of the circuit (100 ml) or static control (20 ml) while adjusting for volume lost while taking samples.

Platelet Assay: A platelet assay previously described (Baker et al, Am. J. Hematol. 56(1):17-25 (in eng) (1997)) was modified to isolate and label platelets (PLTs) prior to seeding on Ti surfaces. 1×10⁶ labeled PLTs were added to either bare Ti or EPC-coated Ti, followed by incubation (10 min, 37° C., 5% CO2). Surfaces were rinsed ×3 with PBS and imaged with a fluorescent microscope.

In order to limit platelet activation all steps were performed with solutions on ice. To wash and suspend platelets while minimizing platelet activation, a buffered saline glucose citrate solution (BSGC) were used containing 8.6 mM Na₂HPO₄, 1.6 mM KH₂PO₄, 0.12 M NaCl, 0.9 mM EDTA, 13.6 mM Na₃ Citrate, 11.1 mM Glucose, titrated to a pH of 7.1 in deionized water. All solutions were sterilized via vacuum filtration prior to use.

PLTs were isolated from blood (pooled from 4 pigs) as previously described (Baker et al, Am. J. Hematol. 56(1):17-25 (in eng) (1997)). Briefly, the blood 2:1 was diluted with BSGC in 50 ml conical tubes, centrifuged at 600 g for 3 min at 10° C. with low brake setting. Approximately 10 ml of the lowest density clear platelet-rich plasma layer (PRP) was collected from each sample and pooled. The higher density semi-clear layer (approximately 25 ml) from each tube was diluted 1:1 with BSGC in 50 ml conical tubes and centrifuged at 600 g, 5 min, 10° C. and low brake setting. The resulting supernatant was again collected, diluted 1:1 with BSGC, centrifuged at 600 g, 4 min, 10° C. and low brake setting. After these two ‘wash steps’ the PRP was collected and pooled with the PRP from the initial step. The combined PRP was subsequently centrifuged at 1600 g for 10 min at 10° C. with low brake setting. The resulting pellet of platelets was resuspended in 1 ml BSGC, labeled with 5 μl of CTO (5 μl of a 1 mM CTO solution) by incubating at 25° C., 45 min on the lowest setting of a rocker (BioRocker Model 110, Denville Scientific) shielded from light. Following, the solution was centrifuged again (1310 g, 10 min, 25° C.), the supernatant discarded and the fluorescently labeled platelets gently resuspended in their initial volume of BSGC. Platelets were kept in constant motion as above at 25° C., shielded from light, until used for experiments (within 4 hrs of isolation). The concentration of platelets in solution was determined as 1.062×10⁹/ml with a Complete Blood Count Analyzer (Abbott Cell Dyn 3700 Hematology System, Abbott Diagnostics).

EPCs were grown to confluency and fluorescently labeled (with a 1 mM solution of Cell Tracker Green) as described above. A solution of 1×10⁶ platelets labeled with Cell Tracker Orange was then suspended in a 10 ml, 1:1 solution of DMEM/Tyrodes buffer (20 mM Hepes, 137 mM NaCl, 2.7 mM KCl, 1 mM CaCl2, 11.9 mM NaHCO₃, 5.5 mM Glucose) to activate platelets. 5 ml of platelet-activating solution was added to either bare Ti or EPC-coated Ti, followed by incubation (10 min, 37° C., 5% CO₂). Surfaces were then rinsed ×3 with PBS prior to imaging. 5 images along the Ti slide (100×) were obtained. The experiment was repeated ×3.

Statistics: The mixed model (SAS, PROC MIXED) was used to compare the longitudinal data of alamarBlue reduction on FN-precoated glass surfaces and Ti, as well as nitric oxide production between the groups Ti or FN-glass, and high shear, physiological shear or static conditions. To compare platelet adhesion to EPC-coated and uncoated Ti, a T-Test (SAS, PROC TTEST) was applied. EPC spreading (area) and elongation (roundness) under flow were evaluated with the Kruskal-Wallis Test (SAS, PROC NPAR1WAY). Cell roundness between the different surfaces' (Ti and FN-glass) and flow conditions (15 dynes/cm² vs. 100 dynes/cm²) was evaluated with Two-Way ANOVA-(SAS, PROC GLM). The Kolmogorov-Smirnov Test (SAS, PROC NPAR1WAY) was used to compare cell alignment under static conditions (pdf found to be uniformly distributed between 0 degree—and 90 degree angles), physiological—and supraphysiological shear stresses on Ti. The significance level was assumed to be 0.05 for all tests.

### Results

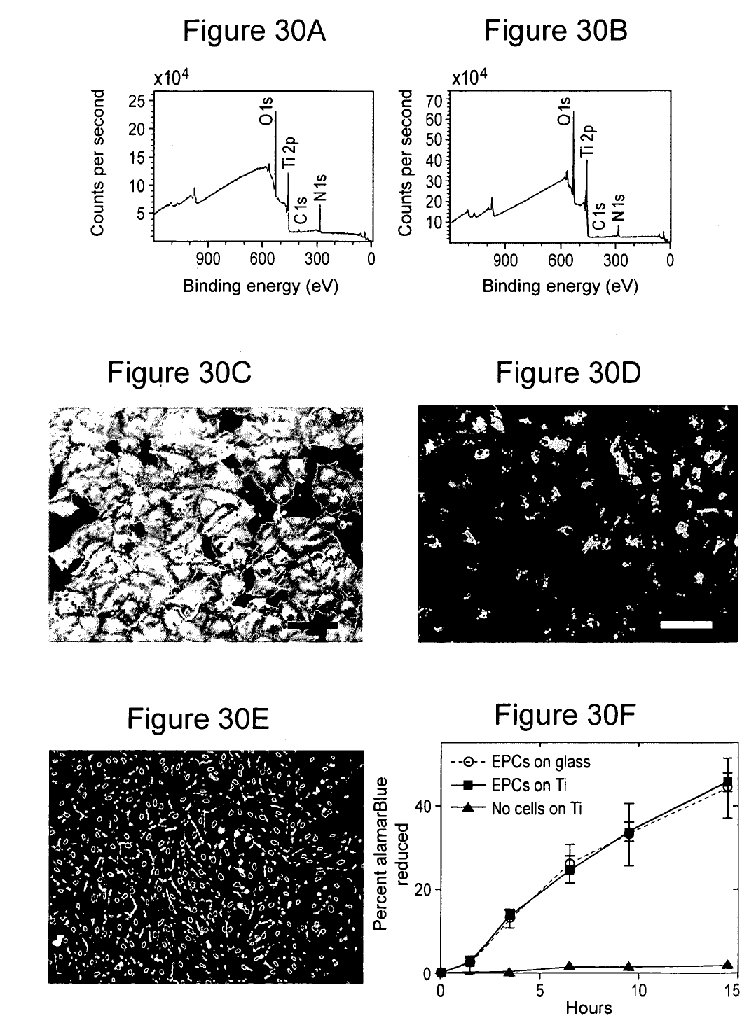

Ti Surface Composition: X-ray photoelectron spectroscopy (XPS) indicated that the chemical composition of the titanium oxide microsphere surface of the HeartMate II ventricular assist device (Thoratec) was almost identical to the manufactured Ti surfaces (FIGS. 30A and 30B).

Ti Surface Topography: The topography of our Ti surfaces was assessed with atomic force microscopy (AFM). The average roughness (R_{q}) was approximately 10 nm (see FIG. 34). The Ti surface gave a contact angle of 42.3±1.98° (n=5), which is consistent with a Ti oxide film deposited in the absence of oxygen flow (Lin et al, Biomed. Mater. 4(1):015013 (2009)).

EPC Isolation: Porcine EPC colonies appeared 5-7 days after isolation and had an average of 1 colony per 43 ml blood. Isolated EPCs exhibited characteristic cobblestone endothelial cell morphology by light and scanning electron microscopy (SEM) (FIG. 30C), took up DiI-labeled acetylated Low Density Lipoprotein (DiI-Ac-LDL) (FIG. 30D) and stained positive for typical endothelial cell markers such as CD31 (PECAM) (FIG. 30E), endothelial Nitric Oxide Synthase (eNOS) (see FIG. 35), Bandeiraea Simplicifolia-1 lectin, Vascular Endothelial Growth Factor-R2, and von Willebrand factor. Flow cytometry also confirmed presence of CD31 and absence of monocytic markers CD14 and CD45 (see FIG. 36).

EPC Growth on Ti: The maximal seeding density of the EPCs in full EPC medium on Ti was 150,000 cells/cm². The results indicate that EPCs attach to Ti surfaces and adhere very well when compared to fibronectin-precoated glass (FN-glass). If seeded below this density, EPCs divide and grow on Ti under static conditions until they form a confluent monolayer, fully covering the entire surface. At that point, EPCs are contact-inhibited and remain viable in long term culture on Ti (>1 week).

EPC Viability on Ti: A significant difference was not found in metabolic activity between confluent layers of EPCs on either Ti or FN-glass surfaces in long term culture (>1 week) using the alamarBlue redox assay (Invitrogen) (p=0.42, n=6) (FIG. 30F). Therefore, EPCs on uncoated Ti remain as viable as those growing on FN-glass surfaces under long term culture conditions.

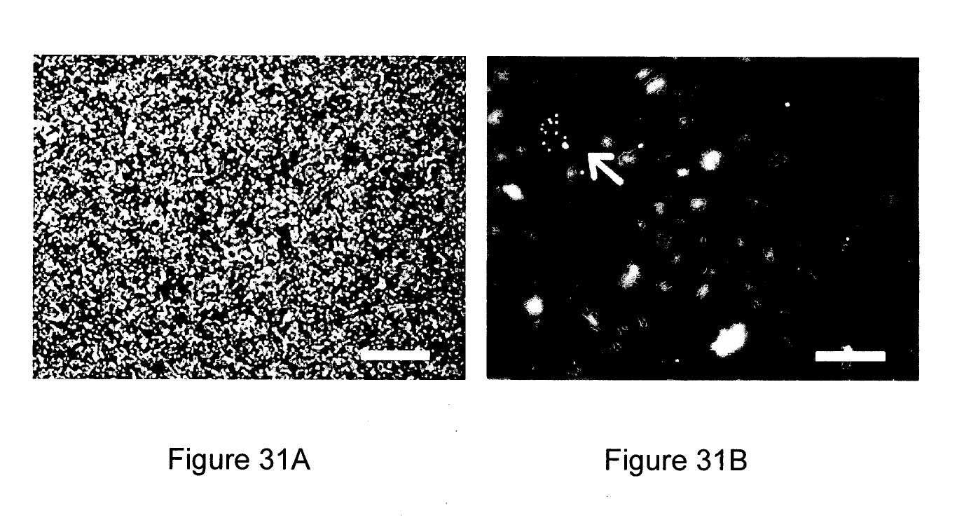

Platelet Adhesion: EPC-coated Ti slides and bare Ti slides (controls) were incubated for 10 min at 37° C. under identical conditions with fluorescently-labeled platelets, pooled from 4 different pigs. A>500-fold difference was found in platelet (PLT)-adhesion to EPC-coated vs. uncoated surfaces (2.5±0.3×10⁶ PLTs/cm² adhered to uncoated Ti (FIG. 31A), but only 4600±2000 PLTs/cm² to EPC-coated Ti (FIG. 31B), p<0.0001).

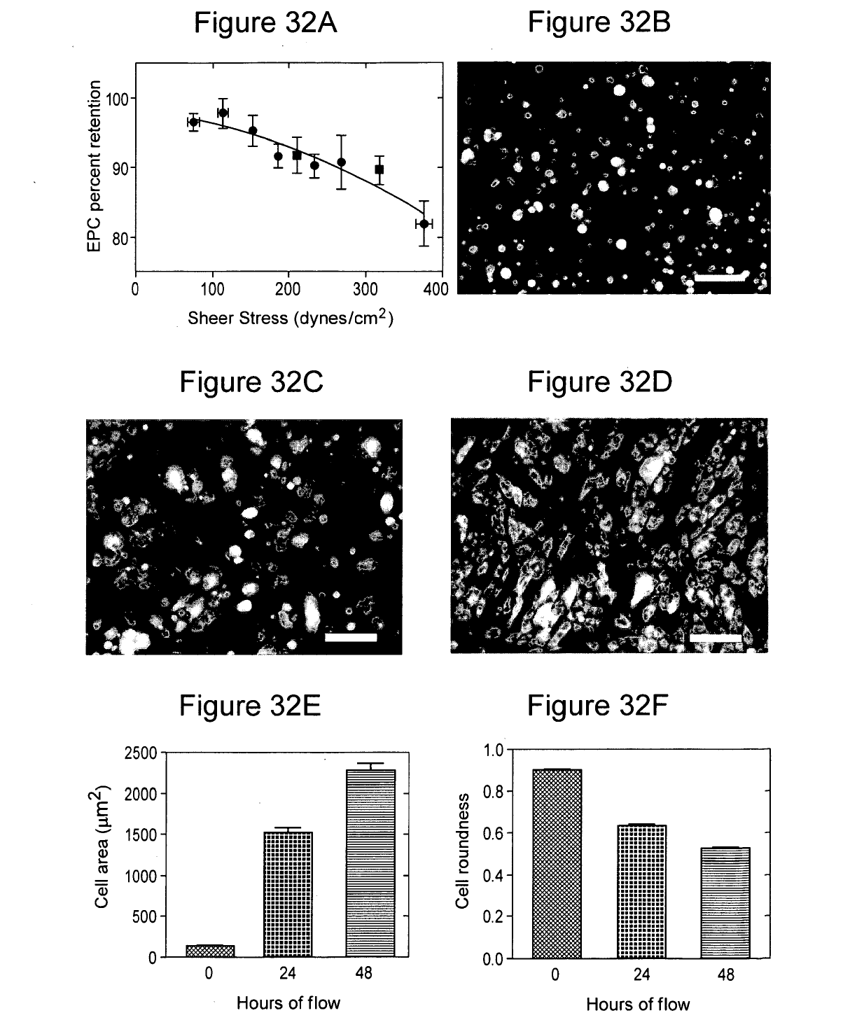

EPC Adhesion under Shear Stress: EPCs remained adherent to Ti after only 15 min settling time in saline solution without precoating of the Ti surface, even under shear stresses exceeding 20-fold normal physiological conditions, which are of the magnitude of 15-20 dynes/cm² in the human arterial system (Truskey et al, Transport Phenomena in Biological Systems, ed Horton M J (Pearson Educaton, Inc., Upper Saddle River, N.J. 07458), pp. 103-105 (2004). At a shear stress of approximately 200 dynes/cm², over 90% of cells were retained on the Ti surface and at the highest shear stress of 380 dynes/cm² over 80% of EPCs still remained adherent (FIG. 32A).

EPC Spreading, Retention and Alignment on Ti Following Exposure to Flow: EPCs isolated from 3 different pigs were used in separate experiments and seeded on Ti where they were left to settle in serum free medium for only 15 minutes prior to initiation of flow. EPCs spread on Ti while being subjected to continuous physiological shear stress (48 hrs) until forming a confluent monolayer (FIGS. 32B-32D). Their area significantly increased from 124.9±5.8 μm² to 1524±56 μm² after 24 hrs and to 2279±81 μm² after 48 hrs (FIG. 32E) (p<0.0001). At that time, EPCs occupied>96±1.2% of the Ti surface. Whereas almost perfectly round at the beginning of flow (roundness of 0.896±0.004), they progressively elongated during flow (roundness=0.63±0.01 after 24 hrs, and 0.523±0.00751 after 48 hrs, p<0.0001, FIGS. 32B-32D and 32F).

EPCs grown to confluence on Ti×24 hrs prior to flow and then exposed to shear stress, also maintained an intact monolayer under long term exposure (48 hrs) to physiological—(15 dynes/cm²) and supraphysiological shear stress (100 dynes/cm²). EPCs elongated under physiological shear as compared to static culture conditions (roundness=0.688±0.004 vs. 0.426±0.007, p<0.0001), and even more so under supraphysiological shear stress (roundness=0.369±0.005, p=0.001) (FIGS. 33A-33C). Further, EPCs on Ti aligned with the direction of flow under 15 dynes/cm² (angle=16.9±0.2°), which was significantly different from a random distribution under static conditions (angle=44.8±0.5°, p<0.0001), and even closer alignment with the direction of flow under 100 dynes/cm² (angle=12.4±0.3°, p<0.0001) (FIG. 33D).

Nitric Oxide Production: EPCs from 4 different pigs in 24 independent experiments produced significantly more NO during 48 hrs of physiological shear stress than during static conditions (p<0.0001), and under supraphysiological shear as compared to physiological shear (p=0.0001) (FIG. 33E). There was no statistically significant difference in NO production between Ti and FN-glass (p>0.85).

In summary, the benefit of in vitro seeding of artificial blood-contacting surfaces with autologous endothelial cells has been demonstrated by Zilla et al more than a decade ago, when he successfully reduced the occlusion of femoropopliteal polytetrafluoroethylene bypass grafts (Zilla et al, Ann. Vasc. Surg. 19(3):540-548 (1994), Leseche et al, Ann. Vasc. Surg. 9 Suppl:S15-S23 (1995), Magometschnigg et al, J. Vasc. Surg. 15(3):527-535 (1992), Deutsch et al, Surgery 126(5):847-855 (1999), Meinhart et al, Asaio J. 43(5):M515-521 (1997)). However, all clinical utilization of this strategy was hampered by the difficulties obtaining the endothelial cells through an additional invasive procedure, such as harvesting a healthy native vessel. Other approaches to isolate cells have included isolation of EPCs from bone marrow, which requires an invasive, and painful bone marrow aspiration (Reyes et al, J. Clin. Invest 109(3):337-346 (in eng) (2002)), and the culture of microvascular endothelial cells from dermal tissue. The latter has remained problematic as well, because of difficulties in isolating a pure cell population without fibroblast contamination, low endothelial cell yields, and a short lifespan of the isolated cells (Richard et al, Exp. Cell Res. 240(1):1-6 (in eng) (1998)). Since peripheral blood is readily and easily available, blood-derived EPCs may be an ideal cell type to seed onto implantable cardiovascular devices, such as Nitinol stents, which are Ti-coated, or Ti-based MCADs.

In order to translate this technology into clinical practice, certain prerequisites must be met: (i) EPCs should adhere to and grow on Ti surfaces, ideally without precoating the surface with a potentially prothrombotic protein layer, (ii) EPCs must adhere under shear stress on such surfaces, and (iii) EPCs must provide for a more biocompatible surface, e.g. inhibit platelet adhesion and activation, and also release nitric oxide in analogy to healthy native endothelium.

Breithaupt et al (Head Face Med. 4:14 (2008)) and Yeh et al (J. Biomed. Mater. Res. A. 76(4):835-841 (in eng) (2006)) reported that human umbilical vein endothelial cells (HUVECs) can grow on Ti-without surface treatment under static conditions. The above-described results show that blood-derived EPCs in serum free medium adhere within minutes of seeding to non-functionalized Ti. This has the distinct advantages of avoiding activation of the coagulation cascade through exposure of prothrombotic protein coatings (collagen or fibronectin, etc.), as well as potential sensitization of the immune system against non-autologous proteins. Further, uncoated devices are easier to manufacture and less costly.

Surface roughness may be an important factor for ideal EPC growth conditions and adhesion under shear stress. Chung et al (Biomaterials 24(25):4655-4661 (in eng) (2003))\ compared adhesion of endothelial cells on fine grain vs. coarse grain Nitinol particles and found best results for 60 nm rather then 100 nm particles. Samaroo et al (Int. J. Nanomedicine 3(1):75-82 (2008)) studied HUVEC adhesion to modified polyurethane surfaces of different roughness and found best adhesion for a mean roughness Rₐ of 35 nm. Yet, in neither study had cells been exposed to physiological shear stress. In the studies described above, it was found that the very smooth Ti surface (R_{q}=10 nm) provides for excellent EPC adhesion under physiologic and supraphysiologic shear stresses. More than 80% of EPCs are still retained at shear stresses 25-fold higher than physiological conditions. It is believed that such exceptionally strong adhesion to uncoated Ti has never been reported. Only recently, similarly strong adhesion has been described for human endothelial cells and human umbilical cord blood-derived EPCs grown on smooth muscle cells (Wallace et al, Ann. Biomed. Eng. 35(3):375-386 (in eng) (2007)), as well as FN-precoated surfaces (Brown et al, Tissue Eng. Part A 15(11):3575-3587 (in eng) (2009)). This is attributed to the unique characteristics of the naturally formed titanium oxide film of Ti. Yeh et al also found that HUVECs grow significantly better on titanium oxide as compared to other metals *Yeh et al, J. Biomed. Mater. Res. A 76(4):835-841 (in eng) (2006)). Further, Lin et al recently described an increase in cellular activity of porcine aortic smooth muscle cells as a function of increased oxygen content of Ti surfaces synthesized by electron-beam evaporation at different oxygen flow rates (Lin et al, Biomed. Mater. 4(1):015013 (2009)).

Given EPCs' exceptionally strong adhesion to Ti and the fact that they can spread to a monolayer under physiological flow conditions and EPCs' ability to adapt to their environment by elongating and aligning in the direction of flow, it is theoretically possible to quickly (within minutes) seed a titanium oxide-coated device immediately prior to implantation, on the operating room or catheterization laboratory table. The possibility of such quick seeding times is an important milestone to translate EPC-seeding technology into clinical practice since longer 2-step seeding procedures that require ex vivo culture time have not proven to be practical.

Another important marker of endothelial cell functionality and health is the synthesis of NO, which also inhibits platelet adhesion in vivo (Achneck et al, Vascular 16 Suppl 1:S6-S13 (in eng) (2008)). However, the short half-life of NO in aqueous solutions renders it impractical for direct measurement and has led to the use of its primary oxidation products as surrogates for NO. Previous studies in human blood have shown direct measurements of plasma nitrite to be the most useful marker of vascular NO production due to its relatively low background concentrations (70-140 nM) and detection limits (<5 pm) Lauer et al, Proc. Natl. Acad. Sci. USA 98(22):12814-12819 (in eng) (2001), Allen et al, Free Radic. Biol. Med. 38(9):1164-1169 (2005), Kleinbongard et al, Free Radic. Biol. Med. 35(7):790-796 (in eng) (2003)). Other studies in this field have examined NOX (nitrite plus nitrate) using the Griess assay, but plasma nitrate or NOX are in the 10 μM range in human plasma and have shown little relationship to endothelial function in vivo in humans (Allen et al, Nitric Oxide 20(4):231-237 (in eng) (2009)). Additionally, the Griess assay chemically converts nitrate to nitrite for measurement and has a limit of detection of 2.5 μM in comparison to <5 pM for the Sievers Nitric Oxide Analyzer (NOA 280, Sievers Instruments). Consequently, the direct measurement of nitrite as a marker of NO production by the EPCs has been selected for use.

Since thromboemboli formation is initiated by platelet adhesion to foreign materials (Arvidsson et al, Biomaterials 28(7):1346-1354 (in eng) (2007)), an investigation was made as to whether an EPC-coated Ti surface would decrease platelet adhesion (Thor et al, Biomaterials 28(6):966-974 (in eng) (2007)). An evaluation was made of the propensity of platelets to adhere to EPC-coated Ti surfaces under—the most extreme condition—static incubation in calcium-rich Tyrodes Buffer at 37° C. Still, only a few platelets were observed adhering to the EPC-coated surfaces, whereas>590-fold more platelets adhered to uncoated Ti surfaces under identical conditions.

The interaction of EPCs (on Ti) with the native vascular environment in a stented vessel, for instance, has not been examined. While it cannot be ruled out that EPCs would inappropriately stimulate vascular smooth muscle cell (SMC) proliferation, it seems very unlikely, given the flow mediated increase in NO production observed and the confluent monolayer formed. The vasoprotective properties of NO have been well documented and are comparable to healthy endothelium. They include inhibition of SMC proliferation and migration (Zuckerbraun et al, Circulation 121(1):98-109 (2010)). Further, porcine EPCs may behave differently than human EPCs. However, results have been reproduced with human EPCs from a patient with vascular disease and a healthy human volunteer.

Specifically, after obtaining consent from a healthy volunteer (60 year old female) and patient with documented coronary artery disease (59 year old female), EPCs were isolated following the protocol described above and seeded onto Ti slides at a density of 150,000 cells/cm²×24 hours (37° C., 5% CO₂, in a humidified incubator). EPC seeded Ti slides where then exposed to shear stresses of 15 dynes/cm² and 100 dynes/cm² in the flow circuit. Identical Ti slides with EPCs were kept incubated under static conditions and served as controls for flow. Comparable to the findings reported with porcine EPCs, human EPCs (from healthy donor and diseased patient) were found to adhere well to the Ti slides under physiological and supraphysiological shear stresses, and align and elongate in the direction of flow (see FIG. 38).

The results pave the way for a porcine animal model, which is the most established large animal model for researching biocompatibility of implantable devices (Kang et al, Thromb. Haemost. 89(2):256-263 (2003)).

In conclusion, titanium is used in a variety of implantable cardiovascular devices, however, it is recognized as ‘foreign’ by the body's coagulation system. Through contact activation (Arvidsson et al, Biomaterials 28(7):1346-1354 (in eng) (2007)), it can give rise to thrombosis and emboli, which cause significant morbidity and mortality in patients with these devices, e.g. occlusion of a stent. The studies described above demonstrate that (1) it is feasible to grow blood-derived EPCs on Ti surfaces without any adsorbed proteins, (2) that EPCs grow to a confluent monolayer on Ti under static conditions and physiological flow after only a few minutes prior adhesion time, (3) that. EPCs adhere extremely well to such surfaces, even under supraphysiological shear stresses. It has been further demonstrated (4) that EPCs on smooth Ti surfaces functionally adapt to their environment under flow, produce NO, dependent on the magnitude of shear stress stimulation, and (5) dramatically reduce platelet adhesion when compared to uncoated Ti surfaces. Therefore, it is possible to quickly seed and coat Ti blood-contacting surfaces, such as Nitinol stents and mechanical assist devices, with peripheral blood-derived EPCs just prior to implantation into the cardiovascular system, and thus ameliorate the common complications of platelet adhesion and thrombus formation.

### Example 7

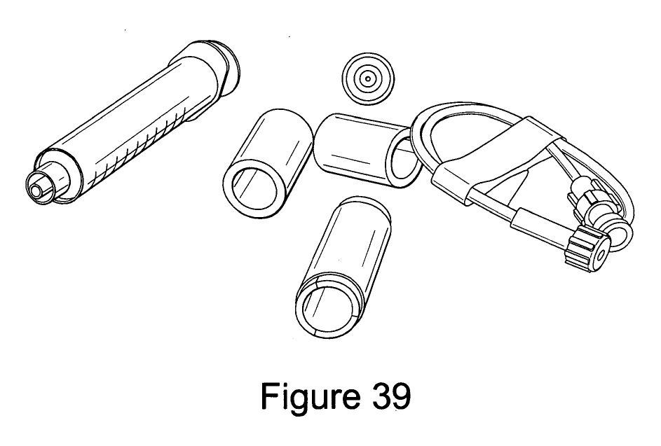

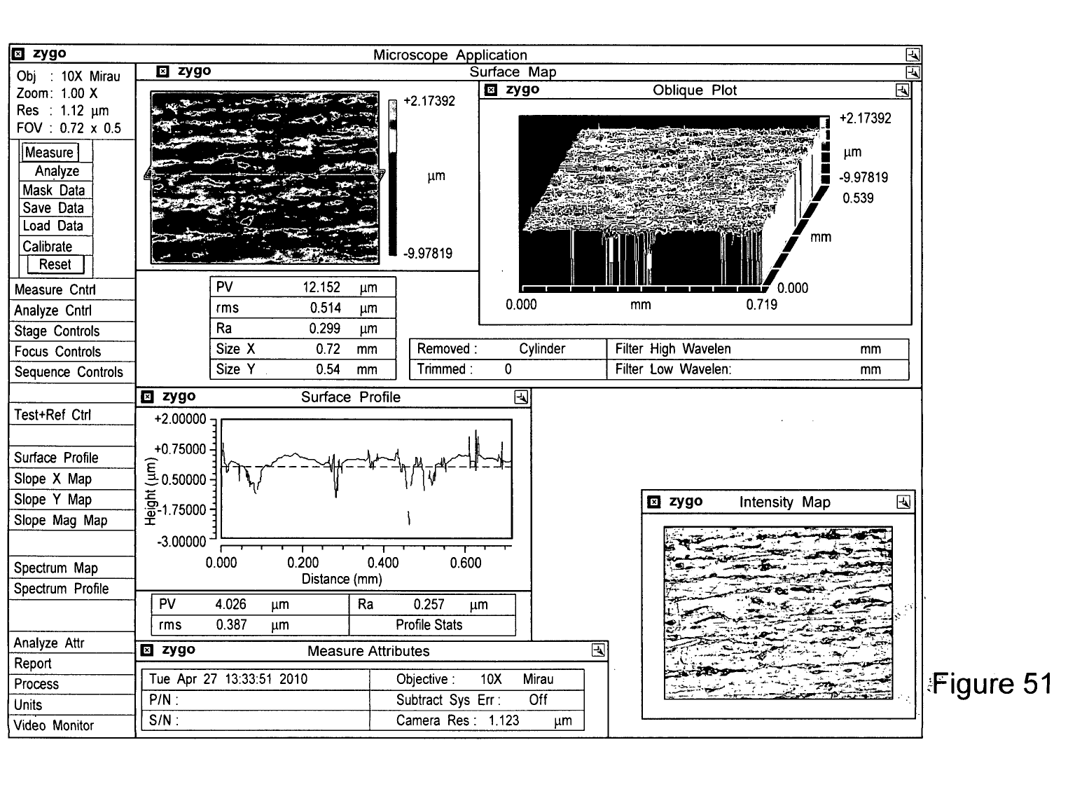

Compound pure titanium (Ti) tubes of average roughness 350 μM (see FIG. 51) were assembled and prepared for implantation as follows. Ti tubes were precut into 3 longitudinal sections and cleaned by submersion in aqua regia (concentrated nitric acid and concentrated hydrochloric acid at a molar ratio of 1:3)×5 min, followed by sonication in soap solution (Alconox powder, diluted 1:100)×5 min, followed by sonication in deionized water ×5 min. After 30 more rinses with deionized water, Ti sections were air-dried under a laminar flow hood. Meticulous cleaning is necessary to remove environment-derived carbon and nitrogen contaminants from the titanium surface since the best EPC-adhesion has been found to pure titanium surfaces with a high titanium-oxide percentage (as evaluated by x-ray photoelectron spectroscopy). Such cleaned Ti sections were assembled back into a Ti tube in a dust-free environment (under a laminar flow hood) by heat-shrinking medical grade polyvinyl chloride (PVC) heat-shrink tubing around the Ti pieces. Assembly was facilitated by placing the 3 individual Ti sections on a mandrel. A heat gun was used to heat-shrink the PVC tubing around the Ti. (See FIG. 39.)

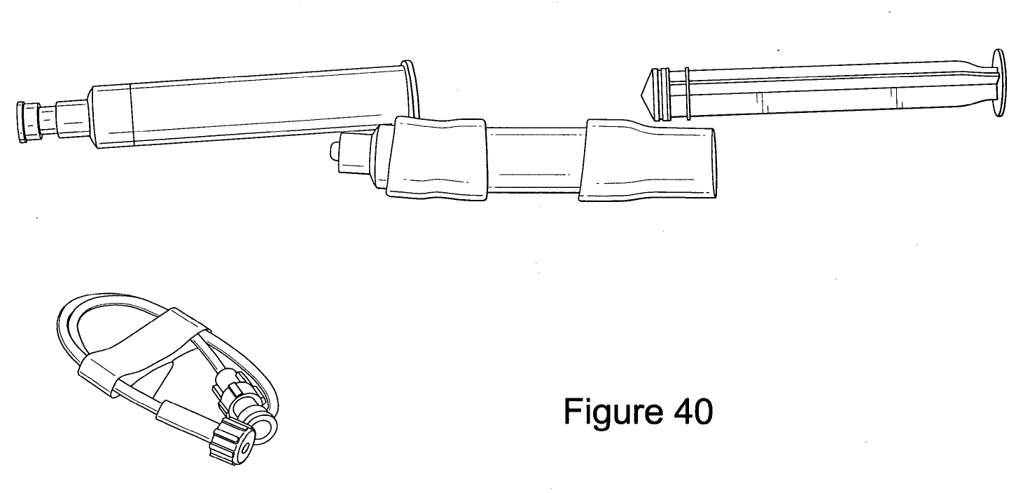

In order to fill the Ti tube with an EPC-containing solution, each end of the Ti tube was inserted into approximately 1.5 inch sections of silastic tubing (medical grade silicone rubber tubing, 17 mm outer diameter, 12.7 mm inner diameter) and the cut-off end of a 10 ml syringe was inserted into one tubing section (show on the left end of the Ti tube in FIG. 40). All parts were gas-sterilized (ethylene oxide) before use. At the time of seeding, a 10 ml syringe was filled with fluorescently labeled EPCs in serum-free EPC medium (EBM-2 with EGM-2, Lonza/Clonetics) and inserted into the free tubing end on the tube's opposite side (shown on the right end of the Ti tube in FIG. 40). The device was then held vertically and upon pressing the piston of the 10 ml syringe, the EPC-containing solution was forced into the Ti tube while air escaped through the other end of the Ti tube, more specifically through the cut-off syringe tip inserted in the silastic tubing. Once the entire lumen of the Ti tube had been filled with EPC-containing solution, the lure lock of the cut-off syringe tip was closed with a stop cock. Without removing the 10 ml syringe inserted into the silastic tubing section of the Ti tubing, the entire device was placed inside a sterile cover (a sterile glove was used) and inserted into the (non-sterile) seeding device. The use of serum-free medium was found to be important for this seeding step, since serum (albumin) hinders EPC-binding to the metal surface during the first minutes of seeding.

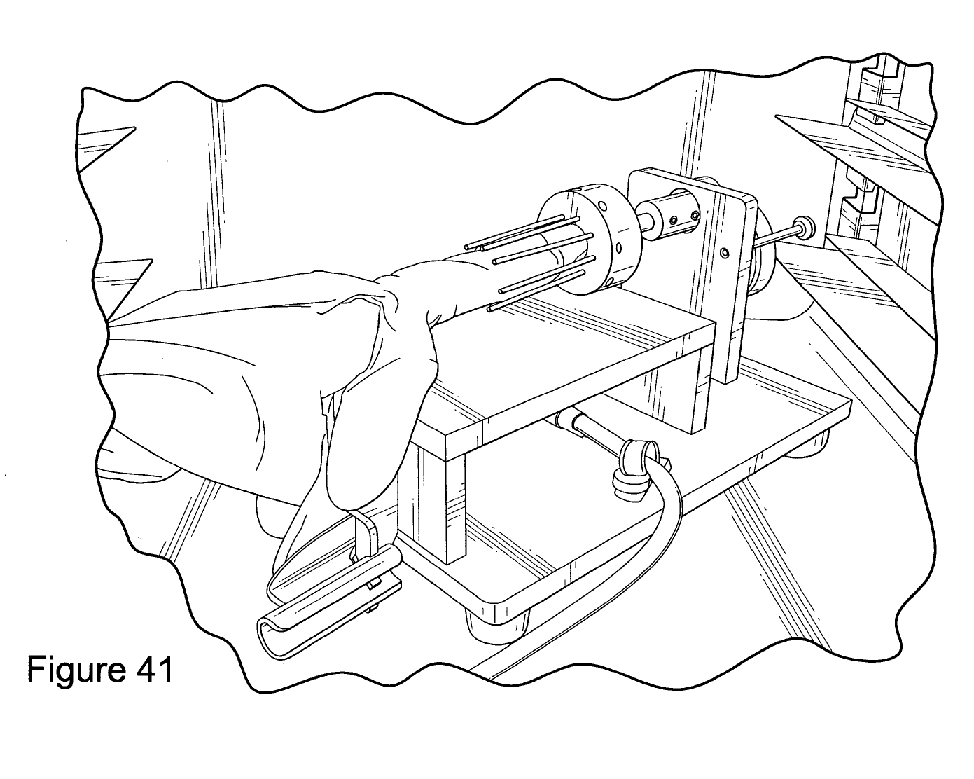

According to the quick-seeding methodology, the device is seeded within minutes, immediately prior to implantation. For this purpose, the Ti tube (now filled with EPC solution and inside a sterile cover) was inserted into the F-16 stainless steel prongs of the rotating polysulfone head of the seeding device (FIG. 41) and placed into an incubator at 37° C. The rotation speed (and seeding time) depends on the size (radius) of the titanium tube or device to be seeded. For this specific example, a Ti tube (inner radius=6.4 mm) was used. Implantation was at the height of the right kidney and, in order to successfully fit the Ti tube with an inner radius of 6.4 mm into the inferior vena cava, it was necessary to ligate the right renal artery and vein. (In subsequent experiments, a smaller Ti tube having an inner radius of 4.7 mm or 3.0 mm will be used.) Best results were achieved with a rotation speed of 10 rotations per hour and a seeding time of 30 min (or 5 rotations total). To seed a smaller-size tube, e.g. with an inner radius of 4.7 mm, a rotation speed of, for example, 13-14 rotations per hour can be used for a seeding time of 22 min, and for a tube of inner radius=3 mm, a rotation speed of, for example, 20 rotations per hour can be used for a duration of 15 min. Optimal seeding results were achieved with an EPC concentration of 1−2×10⁶ cells per ml EPC-medium. Further, it was found that EPCs under these conditions remain viable and functional inside the sealed-off gas impermeable Ti tube for the time periods specified.

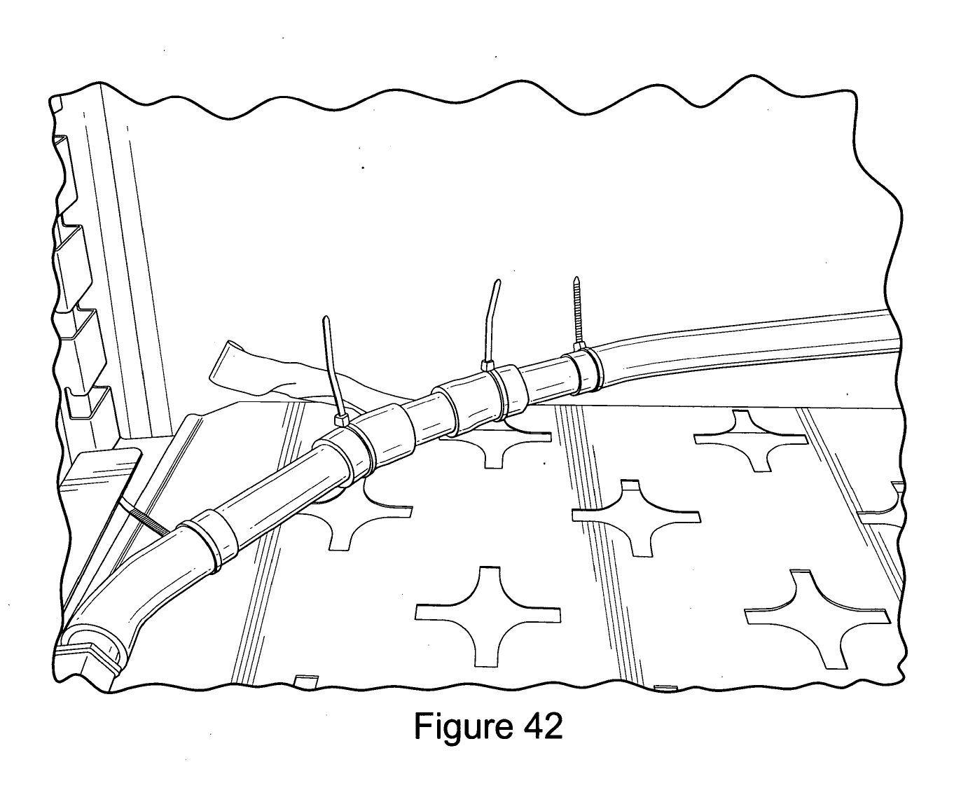

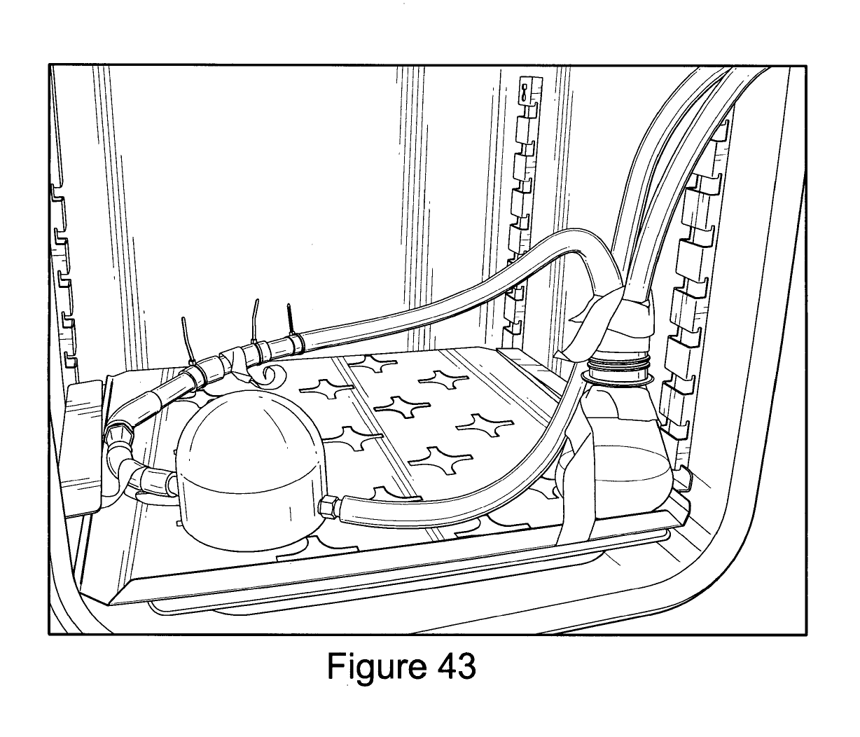

The ability to generate a confluent surface after a short seeding period with immediate exposure to flow was tested ex vivo in a flow circuit as shown in FIGS. 42 and 43. The Ti tube is shown inserted into a flow loop consisting of pulse dampener, reservoir and cardiopulmonary bypass pump (not shown) inside an incubator (5% CO₂, 37° C.). Flow was maintained×48 hours in all experiments. The Ti tube was then removed, opened by cutting the heat-shrink material with a scalpel, and each of three Ti sections imaged with a fluorescent microscope to confirm a confluent endothelial lining.

Ti tubes were implanted into Yorkshire Swine weighing 60 kg. Pigs have a long-standing history as an established animal model for researching the biocompatibility of blood-contacting surfaces (Ueberrueck et al, J. Surg. Res. 124(2):305-311 (2005)). Furthermore, the pig is one of the most accepted models for the study of coagulation biology and inflammation (Velik-Salchner et al, Thromb. Res. 117(5):597-602 (2006), Kang et al, Thromb. Haemost. 89(2):256-263 (2003), Dal Nogare et al, Am. Rev. Respir. Dis. 142(3):660-667 (1990)). Pigs are an excellent model for a proof of principle study, since porcine blood is slightly hypercoagulable compared to human blood (Velik-Salchner et al, Thromb. Res. 117(5):597-602 (2006)). Since it was possible to generate an anti-thrombogenic surface in these pigs, the technology can be expected to succeed in humans as well.

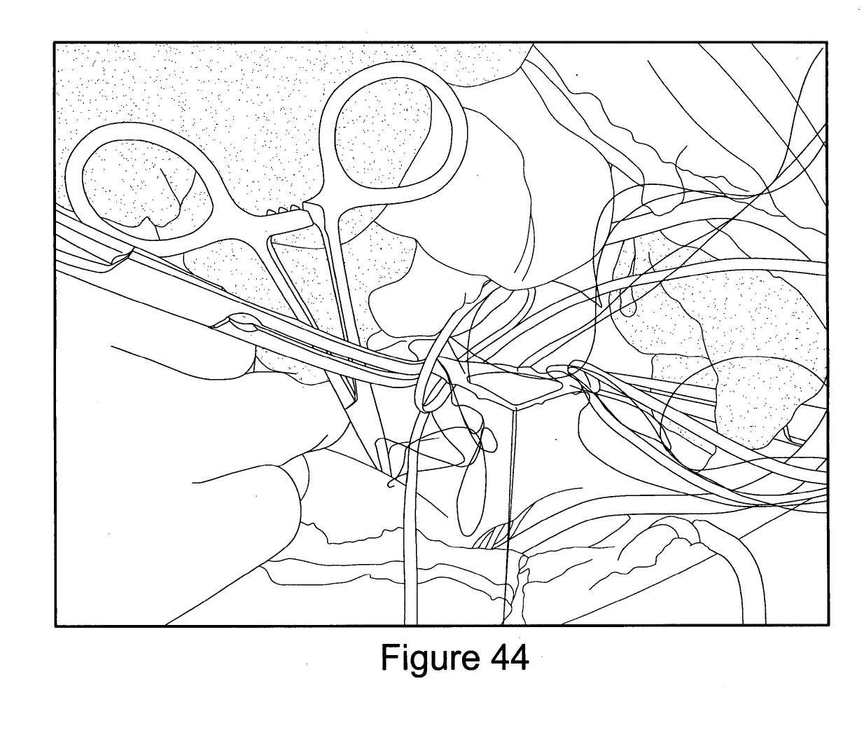

The inferior vena cava was isolated and secured with vessel loops (see FIG. 44) before clamping proximal and distal to the implantation site. A venotomy was performed and the titanium tube inserted. Following, the venotomy was closed with a running 6-0 Proline suture and flow was re-established by removing the vascular clamps. The control pig received a bare (uncoated) Ti implant and the test pig received an EPC-lined Ti implant.

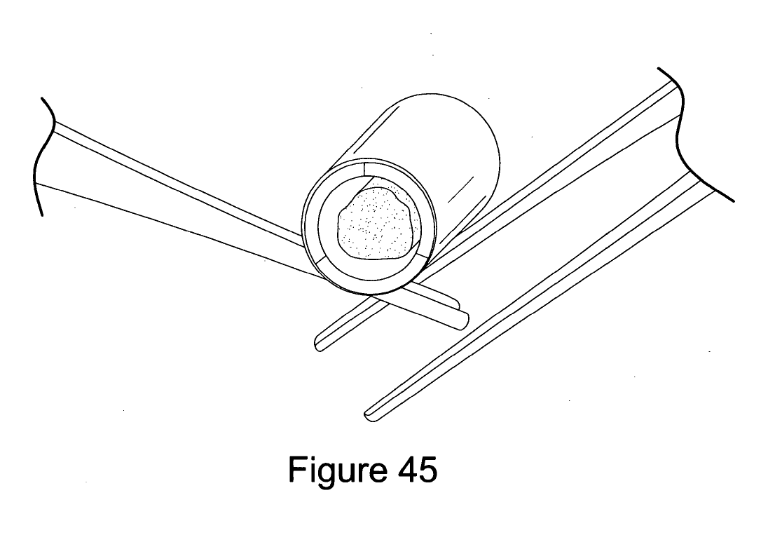

The pig was maintained at normal (porcine) physiological parameters for 2 hours before the Ti tube was explanted. In order to do so, the IVC was cross-clamped proximal and distal to the device, and both ends of the vein cut with heavy scissors. The device with the surrounding vein was removed en-block and immediately photographed on the operating room table. Of note, the entire lumen of the control implant (bare Ti only) was occluded by thrombosis (FIG. 45). After explantation, the animal was euthanized.

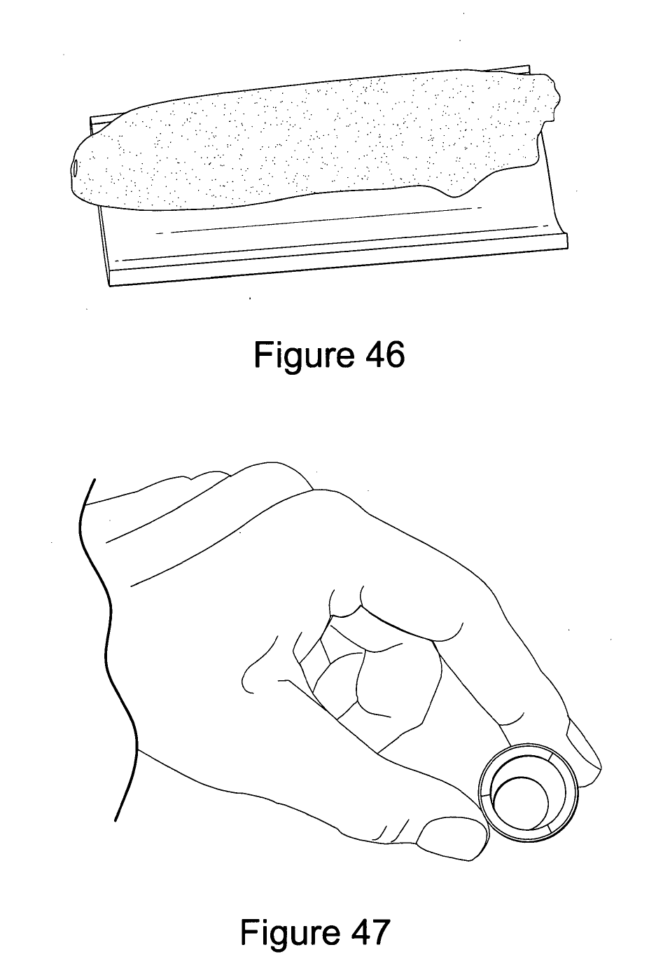

The Ti tube was disassembled into its 3 longitudinally pieces by cutting the heat-shrink tubing with a scalpel. The clot filling the entire lumen is shown in FIG. 46. The same procedure was repeated with the EPC-lined device. As shown in FIG. 47, there was no clot in the EPC-coated device. All experimental conditions between test and control animal were identical.

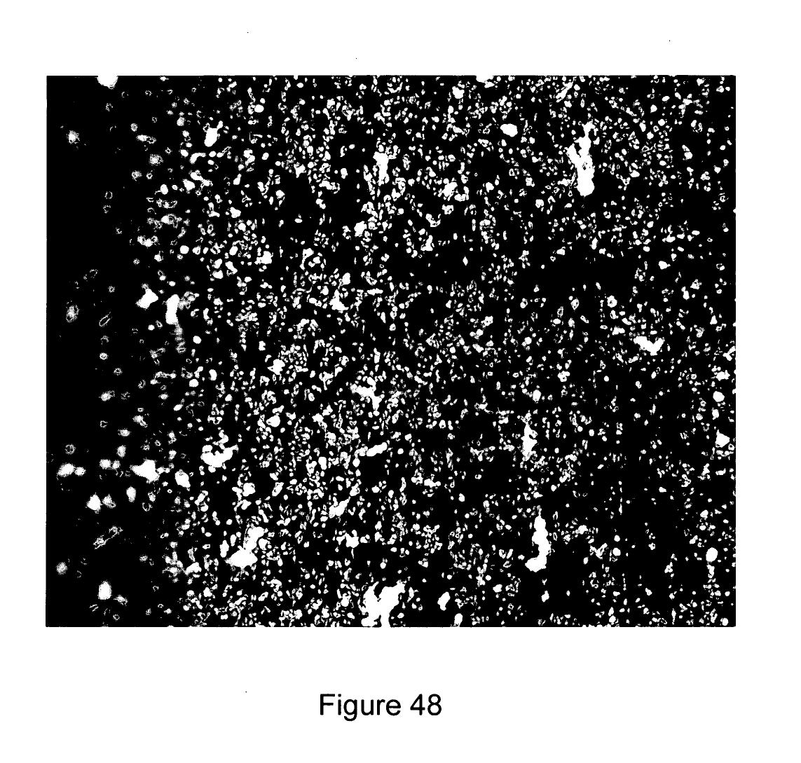

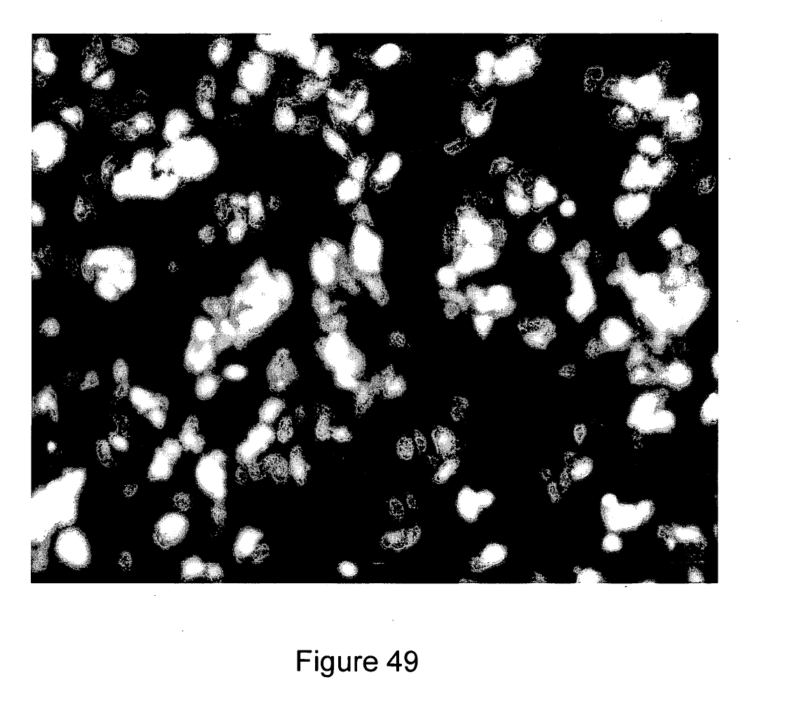

The 3 Ti pieces were washed with phosphate buffered saline solution and fixed in 10% formalin solution. Fluorescent microscopy demonstrated presence of the fluorescently labeled cells at the same density they were seeded prior to implantation (FIG. 48, image at 40×). Furthermore, it was observed that cells had started to spread in vivo, which is apparent by their increased surface area and decreased roundness (FIG. 49, image at 200×).

### Example 8

EPCs adhere remarkably well to Ti surfaces, grow to a confluent monolayer, and are retained under physiologic shear stresses. These discoveries, as well as the development of a modified nitinol stent delivery system that allows for quick (2-3 min) cell seeding, are described above.

Since ˜70% of nitinol stents are currently used on an elective basis, as defined by a time interval of ≧30 days from decision to actual implantation, EPCs can be isolated and cultured in the majority of patients with peripheral arterial disease who are candidates for a stent. Further, the yield of EPCs per blood draw can be significantly increased by administration of AMD3100 several hours prior to the blood draw (Shepherd et al, Blood 108(12):3662-3667 (2006)). AMD3100 increases the success rate of isolating autologous EPCs by 10-fold with only mild side effects reported in a small number of patients (Shepherd et al, Blood 108(12):3662-3667 (2006)). Therefore, it is feasible to isolate EPCs with more than 90% certainty from a single peripheral blood draw.

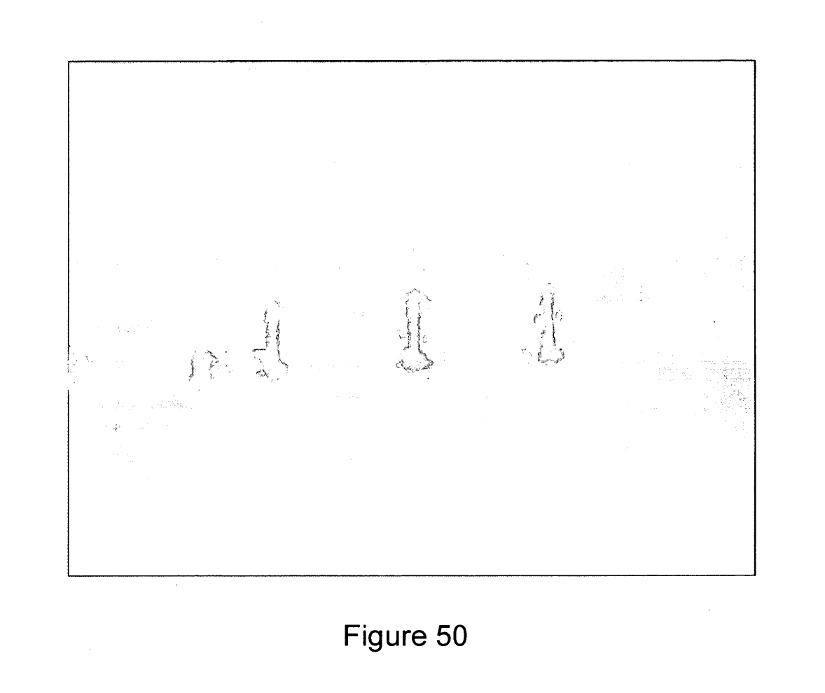

Manufacture stent delivery system/optimize imaging/recompression. A multitude of small holes can be drilled into the stent sheath of a nitinol stent delivery system (Cordis PRECISE) (FIG. 50). Each hole can be smaller than a cell (≦10 μm) but allow for passage of fluid, such that EPCs can be quickly seeded onto the stent struts, as described above. Holes can be drilled with a 355 nm frequency tripled Nd:YAG laser through the plastic sheath at a pitch of 100 μm along the length of the sheath with 36 linear lines of holes around the circumference. EPCs can be visualized on the stent struts by labeling them with Cell Tracker (Invitrogen) and imaging with fluorescent microscopy. In order to reuse the modified stent delivery systems for multiple experiments, the stents can be reloaded by cutting off the delivery system's distal inner catheter tip and forcing the stent through a plastic funnel (pipette tip) while submerged in ice water.

The stent delivery system can be modified such that EPCs can be seeded via pressure-driven flow onto the non-deployed stent. A laser can be used to drill holes in the plastic support sheath. Flow rates necessary to achieve complete seeding of the stent within 2-3 minutes, as well as the pressure drop across the perforated stent sheath, can be calculated and experimentally verified by quantifying non-adherent cells. To improve performance, adjusting the pressure drop and/or drilling more holes into the sheath can be examined.

Imaging EPCs on the deployed stent surface can be optimized with a fluorescent microscope. A determination can be made as to which magnification and positioning results in the most comprehensive method to scan the curved stent struts for adherent cells. If necessary, a stent holder for the microscope stage can be constructed that can move and rotate the stent with respect to the field of view.

To reuse the modified sheaths multiple times for experiments, the stent can be cleaned as previously described for Ti surfaces (Achneck et al, Microsc. Res. Tech. 73(1):71-76 (2010)) and the stent can be recompressed in order to reload it into the modified stent sheaths. For the purpose of recompressing an expanded stent, advantage can be taken of the fact that nitinol becomes malleable in ice water.

Optimize EPC seeding/evaluate cell retention under physiological shear stress ex-vivo. EPCs have been successfully isolated from peripheral blood of humans and pigs according to established protocols (Yoder et al, Blood 109(5):1801-1809 (2007)). These cells have endothelial cell (EC) morphology by light and electron microscopy, and exhibit typical EC markers by immunofluorescence and flow cytometry. Since nitinol stents are coated with a layer of only Ti on their surface to prevent nickel from leaching into the blood, Ti slides were manufactured for experimental purposes. 100 nm of Ti was deposited on piranha cleaned glass slides using a CHA Industries Solution E-beam Evaporation System, and the atomic composition of the samples was confirmed as Ti (titanium oxide) using X-ray photoelectron spectroscopy (Wang et al, Applied Surface Science 253:8507-8512 (2007)). Atomic force microscopy of the Ti revealed a surface roughness of approximately 10 nm, comparing well to the electropolished surfaces of nitinol stents (Thierry et al, Biomaterials 23(14):2997-3005 (2002)).

EPCs adhere very well to such Ti surfaces and grow into a confluent layer fully covering the entire surface. Further, their long term viability (>1 week in culture) on Ti was confirmed with an AlamarBlue bioassay. To evaluate EPC adherence under flow conditions, a variable height flow chamber (Mathur et al, J. Biomed. Mater. Res. A. 64(1):155-163 (2003)) was used to investigate EPC retention at several different shear stresses on a Ti slide for each given flow experiment. The wall shear stress acting on the EPCs was calculated using the following equation: i=6 μQ/wh², where r is the shear stress in dynes/cm², μ is the viscosity, Q the flow rate, w the width of the channel and h its height. Fluorescently labeled EPC were incubated with Ti slides for only 15 min in buffered saline to allow for cells to settle down and barely attach to the Ti surface. Such seeded slides were then inserted into the flow chamber and imaged before and after 5 min of exposure to very high shear stress.

The results indicate excellent EPC adhesion to Ti surfaces at shear stresses up to twenty times the physiologic arterial conditions. Thus, it is feasible to seed nitinol stents with this quick seed method and expect adherence of EPCs. To assess the behavior of a confluent layer of EPCs on nitinol stent surfaces under long term flow, EPCs seeded Ti slides were placed in a parallel plate flow chamber and exposed to continuous flow at arterial shear stress for 48 hours. Images were obtained before and after flow. Media samples were collected to determine nitric oxide (NO) production by measuring the metabolite nitrite by chemiluminescence with an Ionics/Sievers Nitric Oxide Analyzer as described previously (Allen et al, Nitric Oxide 20(4):231-237 (2009)). It was found that the EPC monolayer was retained under physiological shear. Moreover, EPCs adapted to their environment by aligning themselves in the direction of flow and producing significantly more NO under flow than under static conditions (p=0.0008).

In further experiments, fluorescently labeled EPCs can be flushed through the side-port of the stent delivery system at the flow rate determined above. After this seeding process (2-3 minutes), the side-port can be flushed with saline to remove non-adherent cells. The flush solution can be collected to count non-adherent cells. Following, the stent can be expanded and imaged. Image J software can be used to determine cell density per stent strut area. To achieve a maximum number of adherent cells, a determination can be made of the optimal EPC concentration in the seeding solution. The sheath can be imaged to confirm absence of cells stuck in the holes. If the number of holes is insufficient for uniform seeding, additional holes can be drilled into the stent sheath.

To evaluate EPC retention on the deployed stent under shear stress, gas-sterilized stents can be seeded in the modified sheaths with EPCs under conditions as determined above. The seeded stent can be deployed into silicone tubing and inserted into a flow circuit. EPCs adherent to the stent can be subjected to physiological shear. After 48 hours of flow, the stent can be removed by cutting the silicone tubing and imaged as above.

Evaluation of EPC retention in vivo. The carotid arteries of Yucatan miniature pigs (4 mm in diameter) provide for a perfect fit for 5 mm stents. Autologous EPCs can be isolated from pig blood. EPC seeded stents can be implanted percutaneously via femoral artery access and deployed under fluoroscopic guidance with the C-arm (OEC 9800) in the pigs' carotid arteries. Each pig can have a non-seeded bare metal stent implanted in the contralateral carotid artery as a control. Pigs can be sacrificed one hour after implantation, the carotid arteries dissected out and fixed in 3.7% formaldehyde. The stents can be removed and imaged with scanning electron microscopy. All stents can be evaluated for EPC adherence, platelet deposition and thrombus formation. Data can be obtained on cell adhesion under arterial shear stress in vivo.

All documents and other information sources cited above are hereby incorporated in their entirety by reference.

What is claimed is:

1. A medical device implantable into a mammal, wherein said device comprises endothelial cells on a blood-contacting surface thereof.

2. The device according to claim 1 wherein said device has a hollow tubular structure.

3. The device according to claim 2 wherein said device is a stent.

4. The device according to claim 1 wherein said device is a mechanical circulatory assist device.

5. The device according to claim 4 wherein said device is a left ventricular assist device.

6. The device according to claim 1 wherein said device is a titanium or titanium alloy-based device.

7. The device according to claim 1 wherein said cells are genetically engineered to express a protein having anticoagulant or anti-inflammatory properties.

8. The device according to claim 1 wherein said cells are endothelial progenitor cells.

9. The device according to claim 8 wherein said cells are blood-derived endothelial progenitor cells.

10. The device according to claim 9 wherein said cells are derived from peripheral blood, umbilical cord blood or placental blood.

11. The device according to claim 1 wherein said device further comprises a coating on said blood-contacting surface, said coating comprising an agent that increases spreading of said cells, said coating being positioned between said blood-contacting surface and said cells.

12. A method of treating a patient suffering from heart failure comprising implanting into said patient the device according to claim 4 in a manner such that blood circulation in said patient is increased and said treatment is thereby effected.

13. A method of treating a patient suffering from a disease or disorder associated with localized flow constriction comprising introducing into the site of said flow constriction the stent according to claim 2 under conditions such that said treatment is effected.

14. The method according to claim 13 wherein said site of flow constriction is present in a vessel.

15. The method according to claim 14 wherein said stent is a titanium or titanium alloy-based stent.

16. The method according to claim 14 wherein said stent is a nitinol stent.

17. The method according to claim 14 wherein said vessel is an artery or vein.

18. The method according to claim 17 wherein said artery or vein is present in the heart, brain or peripheral circulation of said patient.
